SEQUENCE LISTING (14 sequences)

SEQ ID NO: 1 - protein, 5 aa, synthetic construct
  REGION 1..5: note = Synthetic Sequence
NYWMS

SEQ ID NO: 2 - protein, 17 aa, synthetic construct
  REGION 1..17: note = Synthetic Sequence
DIHGNRGFNYHASWAKG

SEQ ID NO: 3 - protein, 9 aa, synthetic construct
  REGION 1..9: note = Synthetic Sequence
ADDSGSHDI

SEQ ID NO: 4 - protein, 12 aa, synthetic construct
  REGION 1..12: note = Synthetic Sequence
QSSQSVYSAKLS

SEQ ID NO: 5 - protein, 7 aa, synthetic construct
  REGION 1..7: note = Synthetic Sequence
YGSTLAS

SEQ ID NO: 6 - protein, 12 aa, synthetic construct
  REGION 1..12: note = Synthetic Sequence
QGTFYGPDWYFA

SEQ ID NO: 7 - protein, 116 aa, synthetic construct
  REGION 1..116: note = Synthetic Sequence
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 8 - protein, 111 aa, synthetic construct
  REGION 1..111: note = Synthetic Sequence
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 9 - protein, 446 aa, synthetic construct
  REGION 1..446: note = Synthetic Sequence
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
  REGION 1..218: note = Synthetic Sequence
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
  REGION 1..446: note = Synthetic Sequence
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
  REGION 1..218: note = Synthetic Sequence
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
  REGION 1..446: note = Synthetic Sequence
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 218 aa, synthetic construct
  REGION 1..218: note = Synthetic Sequence
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0064947 A1
(43) Pub. Date: Feb. 27, 2025

(54) CYTOTOXICITY TARGETING CHIMERAS FOR FIBROBLAST ACTIVATION PROTEIN-EXPRESSING CELLS

(71) Applicant: Glaxosmithkline Intellectual Property Development Limited, Brentford (GB)

(72) Inventors: Christina Ng Di Marco, Collegeville, PA (US); Matthew Robert Sender, Collegeville, PA (US); Brandon James Turunen, Boston, MA (US)

(21) Appl. No.: 18/812,114

(22) Filed: Aug. 22, 2024

Related U.S. Application Data
(63) Continuation of application No. PCT/IB2023/051748, filed on Feb. 24, 2023.
(60) Provisional application No. 63/314,018, filed on Feb. 25, 2022.

(51) Int. Cl. A61K 47/55; A61K 39/395; A61K 47/60

(52) U.S. Cl. CPC A61K 47/55; A61K 39/39558; A61K 47/60

(57) ABSTRACT

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer.

### CROSS-REFERENCE TO RELATED APPLICATION

This application is a continuation of International Patent Application No. PCT/IB2023/051748, filed Feb. 24, 2023, which claims priority to U.S. Patent Application No. 63/314,018 filed on Feb. 25, 2022; the contents of each of which are incorporated by reference herein in their entirety.

### REFERENCE TO AN ELECTRONIC SEQUENCE LISTING

This application contains a Sequence Listing which has been submitted electronically via Patent Center in XML format and is hereby incorporated by reference in its entirety. Said XML copy, created on Aug. 21, 2024, is named 211812_seqlist.xml and is 20,572 bytes in size.

### FIELD OF THE DISCLOSURE

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer.

### BACKGROUND

Cell-surface proteins and their ligands play key roles in tumor initiation, growth and metastasis. Antibody-based therapeutics have promising properties as drug candidates for these indications due to their selectivity for pathogenic cell-surface targets and their ability to direct immune surveillance to target-expressing tissues or cells to induce depletion of the pathogenic cells. Examples of such depletion mechanisms include antibody-dependent cellular cytotoxicity (ADCC), antibody-dependent cellular phagocytosis (ADCP), and complement-dependant cytotoxicity (CDC). However, antibody-based therapeutics often suffer from a lack of bioavailability, high cost, thermal instability, and difficult manufacturing due to their size, complexity and peptide based structures. Conversely, small molecule therapeutics often provide affordability, stability, and the convenience of oral dosing, but may suffer from poor selectivity and off-target effects, while also lacking the immune control of therapeutic antibodies.

Accordingly, a need exists for improved therapeutic approaches that target pathogenic cells for use in the treatment of disease. Such compositions and related methods are provided in the present disclosure.

### SUMMARY

In one aspect, the present disclosure provides a heterobifunctional molecule referred to as a cytoxicity targeting chimera (CyTaC) or an antibody recruiting molecule (ARM), wherein the ARM comprises a moiety that binds a target cell-surface protein on a cell and a moiety that binds an exogenous antibody. In a further aspect, the ARM comprises a divalent linker that links the target-binding moiety to the antibody-binding moiety. In a further aspect, the target-binding moiety is a fibroblast activation protein (FAP)-binding moiety. In a further aspect, the exogenous antibody is an anti-cotinine antibody, or antigen-binding fragment thereof.

In a further aspect, the ARM is a compound of Formula (I):

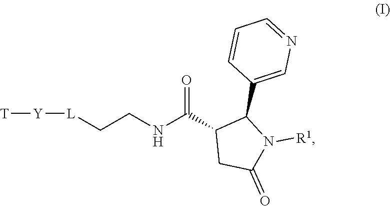

or a pharmaceutically acceptable salt thereof,
wherein:
T is

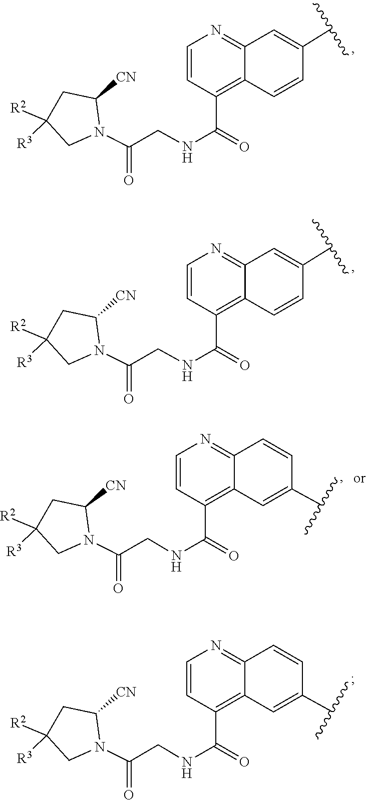

R¹ is C₁₋₄ alkyl or C₃₋₆ cycloalkyl;
R² and R³ are each independently F or H;
Y is a bond or a divalent spacer moiety of one to twelve atoms in length; and
L is a divalent linker as described herein.

In a further aspect, the ARM is a compound of Formula (II):

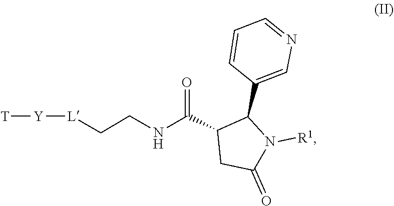

or a pharmaceutically acceptable salt thereof,
wherein:
T is

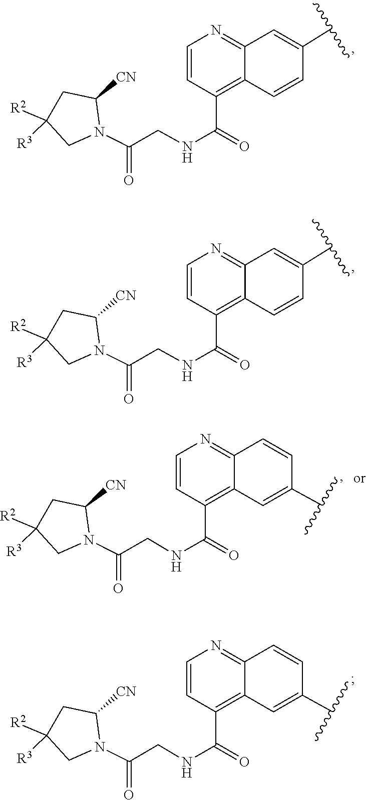

R¹ is C₁₋₄ alkyl or C₃₋₆ cycloalkyl;
R² and R³ are each independently F or H;
Y is a bond or a divalent spacer moiety of one to twelve atoms in length; and
L′ is a divalent linker as described herein.

In one aspect, the present disclosure provides a method of treating and/or preventing a disease or disorder in a patient in need thereof, comprising: administering to the patient a compound of Formula (I) as disclosed herein and an anti-cotinine antibody, or antigen-binding fragment thereof.

In one aspect, the present disclosure provides a method of increasing antibody-dependent cell cytotoxicity (ADCC) of FAP-expressing cells comprising: contacting the cells with a compound of Formula (I) as disclosed herein and an anti-cotinine antibody, or antigen-binding fragment thereof.

In one aspect, the present disclosure provides a method of depleting FAP-expressing cells comprising: contacting the cells with a compound of Formula (I) as disclosed herein and an anti-cotinine antibody, or antigen-binding fragment thereof.

In one aspect, the present disclosure provides a compound of Formula (I) as disclosed herein for use in therapy. In a further aspect, the present disclosure provides a combination comprising a compound of Formula (I) as disclosed herein and an anti-cotinine antibody, or antigen-binding fragment thereof, for use in therapy.

In one aspect, the present disclosure provides a combination comprising a compound of Formula (I) as disclosed herein and an anti-cotinine antibody, or antigen-binding fragment thereof, for use in the treatment of a disease or disorder.

In one aspect, the present disclosure provides use of a compound of Formula (I) as disclosed herein in the manufacture of a medicament for the treatment of a disease or disorder.

In a further aspect, the present disclosure provides use of a combination comprising a compound of Formula (I) as disclosed herein and an anti-cotinine antibody, or antigen-binding fragment thereof, in the manufacture of a medicament for the treatment of a disease or disorder.

In one aspect, the present disclosure provides a combination comprising a compound of Formula (I) as disclosed herein and an anti-cotinine antibody, or antigen-binding fragment thereof.

### BRIEF DESCRIPTION OF THE FIGURES

FIG. 1: Schematic representation of cytotoxicity targeting chimeras (CyTaCs) technology compared to current antibody technology.

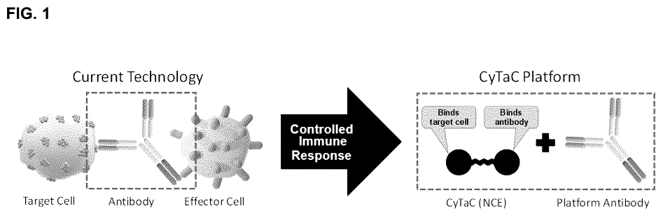

### DETAILED DESCRIPTION

In one aspect, the present disclosure provides a compound of Formula (I):

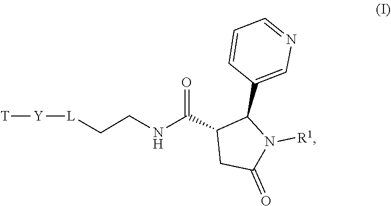

or a pharmaceutically acceptable salt thereof,
wherein:
T is

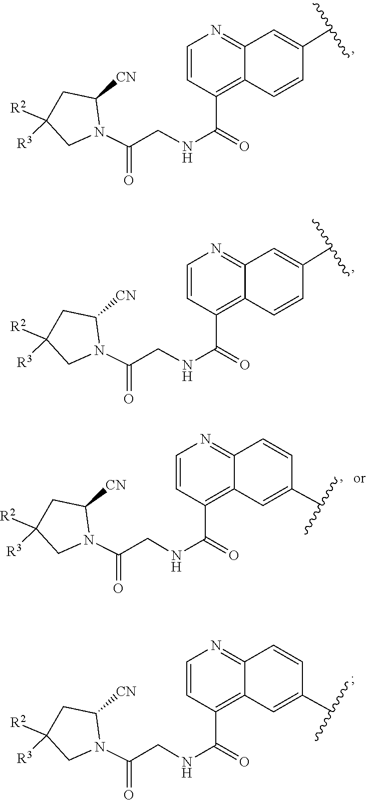

R¹ is C₁₋₄ alkyl or C₃₋₆ cycloalkyl;
R² and R³ are each independently F or H;
Y is a bond or a divalent spacer moiety of one to twelve atoms in length; and
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), (L-n-iv), (L-p), (L-q), or (L-r).

In one embodiment of the disclosure Lisa divalent linker of Formula (L-a):

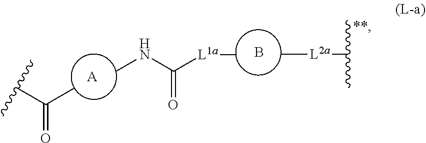

or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

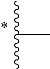

represents a covalent bond to the Y group of Formula (I), or when Y is a bond, a covalent bond to the T group of Formula (I), and

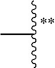

represents a covalent bond to the methylene group of Formula (I).

In another embodiment, Ring A and Ring B of Formula (L-a) are each independently

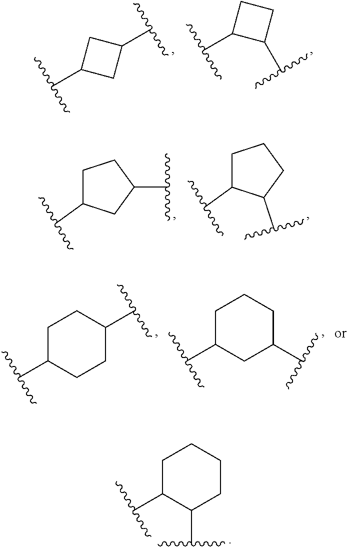

In another embodiment, L is a divalent linker of Formula (L-a-i):

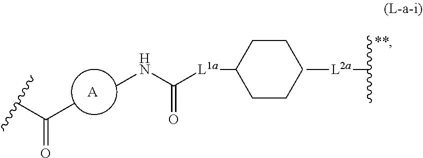

or a stereoisomer thereof,
wherein:
Ring A is C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

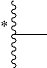

represents a covalent bond to the Y group of Formula (I), or when Y is a bond, a covalent bond to the T group of Formula (I), and

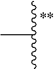

represents a covalent bond to the methylene group of Formula (I).

In another embodiment, Ring A of Formula (L-a-i) is

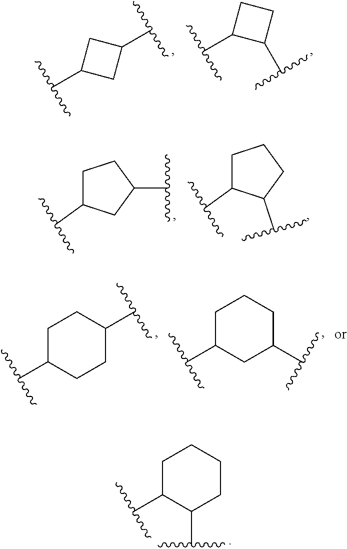

In another embodiment, L is a divalent linker of Formula (L-a-ii):

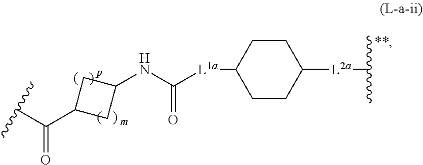

or a stereoisomer thereof,
wherein:
L^{1a} is C₃₋₅ linear alkylene, wherein 1 or 2 methylene units are replaced with —O— or —NR^{a}—;
each R^{a} is independently hydrogen or C₁₋₃ alkyl;
L^{2a} is —O—, —NHC(O)—, or —CH₂—O—;
p is 1 or 2; and
m is 1 or 2;
wherein

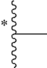

represents a covalent bond to the Y group of Formula (I), or when Y is a bond, a covalent bond to the T group of Formula (I), and

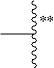

represents a covalent bond to the methylene group of Formula (I).

In another embodiment, L^{1a} of Formula (L-a), (L-a-i), or (L-a-ii) is selected from

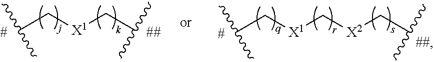

wherein:
j is 1, 2, 3, or 4;
k is 0, 1, 2, or 3;
the sum of j and k is 2, 3, or 4;
q is 1 or 2;
r is 1 or 2;
s is 0 or 1;
the sum of q, r, and s is 2 or 3;
X¹ and X² are independently —O— or NR^{a}; and
each R^{a} is independently hydrogen or C₁₋₃ alkyl;
wherein

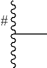

represents a covalent bond to the C(O) group of Formula (L-a), (L-a-i), or (L-a-ii), and

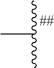

represents a covalent bond to Ring B of Formula (L-a) or to the cyclohexylene group of Formula (L-a-i) or (L-a-ii).

In another embodiment, L^{1a} of Formula (L-a), (L-a-i), or (L-a-ii) is selected from —(CH₂)₂O—(CH₂)₃O—, —(CH₂)₄O—, —(CH₂)₂OCH₂—, —(CH₂)₃OCH₂—, —(CH₂)₂O(CH₂)₂—, —CH₂OCH₂—, —CH₂O(CH₂)₂—CH₂O(CH₂)₃—, —CH₂OCH₂O—, or —CH₂OCH₂OCH₂—. In another embodiment, L^{1a} of Formula (L-a), (L-a-i), or (L-a-ii) is selected from —(CH₂)₂O—, —(CH₂)₃O—, —(CH₂)₂OCH₂—, or —(CH₂)₃OCH₂—. In another embodiment, L^{1a} of Formula (L-a), (L-a-i), or (L-a-ii) is selected from —(CH₂)₂NR^{a}—, —(CH₂)₃NR^{a}—, —(CH₂)₄NR^{a}—, —(CH₂)₂NR^{a}CH₂—, —(CH₂)₃NR^{a}CH₂—, —(CH₂)₂NR^{a}(CH₂)₂—, —CH₂NR^{a}CH₂—, —CH₂NR^{a}(CH₂)₂—, —CH₂NR^{a}(CH₂)₃—, —CH₂NR^{a}CH₂NR^{a}—, or —CH₂NR^{a}CH₂NR^{a}CH₂—, wherein each R^{a} is independently hydrogen or C₁₋₃ alkyl. In another embodiment, L^{1a} of Formula (L-a), (L-a-i), or (L-a-ii) is selected from —(CH₂)₂NR^{a}—, —(CH₂)₃NR^{a}—, —(CH₂)₂NR^{a}CH₂—, or —(CH₂)₃NR^{a}CH₂—, wherein R^{a} is hydrogen or C₁₋₃ alkyl. In another embodiment, L^{1a} of Formula (L-a), (L-a-i), or (L-a-ii) is selected from —(CH₂)₂NH—, —(CH₂)₃NH—, —(CH₂)₄NH—, —(CH₂)₂NHCH₂—, —(CH₂)₃NHCH₂—, —(CH₂)₂NH(CH₂)₂—, —CH₂NHCH₂—, —CH₂NH(CH₂)₂—, —CH₂NH(CH₂)₃—, —CH₂NHCH₂NH—, or —CH₂NHCH₂NHCH₂—. In another embodiment, L^{1a} of Formula (L-a), (L-a-i), or (L-a-ii) is selected from —(CH₂)₂NH—, —(CH₂)₃NH—, —(CH₂)₂NHCH₂—, or —(CH₂)₃NHCH₂—. In another embodiment, L^{1a} of Formula (L-a), (L-a-i), or (L-a-ii) is selected from —CH₂OCH₂NR^{a}—, —CH₂NR^{a}CH₂O—, —CH₂OCH₂NR^{a}CH₂—, —CH₂NR^{a}CH₂OCH₂—, wherein R^{a} is independently hydrogen or C₁₋₃ alkyl. In another embodiment, L^{1a} of Formula (L-a), (L-a-i), or (L-a-ii) is selected from —CH₂OCH₂NH—, —CH₂NHCH₂O—, —CH₂OCH₂NHCH₂—, —CH₂NHCH₂OCH₂—.

In another embodiment, L is a divalent linker of Formula (L-a-iii):

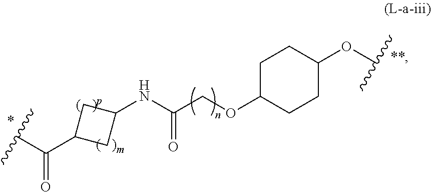

or a stereoisomer thereof,
wherein:
p is 1 or 2;
m is 1 or 2; and
n is 1, 2, or 3;
wherein

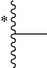

represents a covalent bond to the Y group of Formula (I), or when Y is a bond, a covalent bond to the T group of Formula (I), and

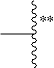

represents a covalent bond to the methylene group of Formula (I).

In another embodiment, L is a divalent linker of Formula (L-a) selected from the group consisting of:

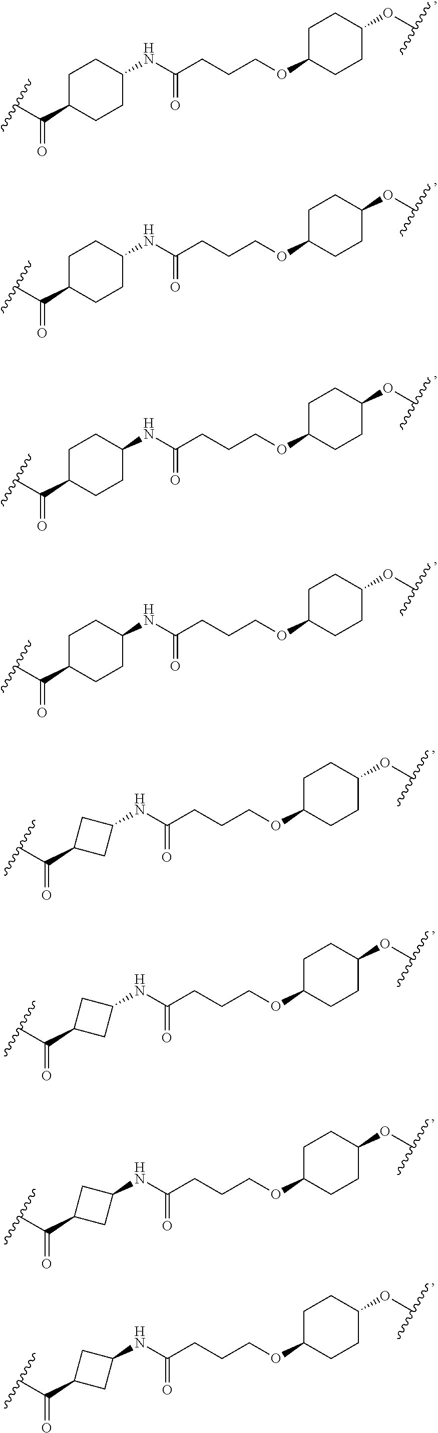

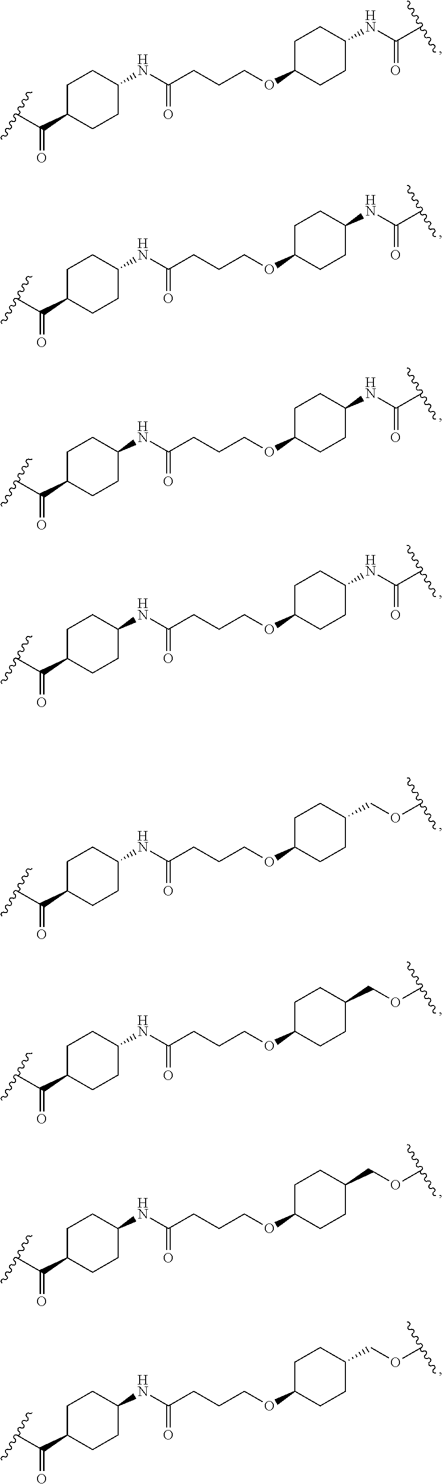

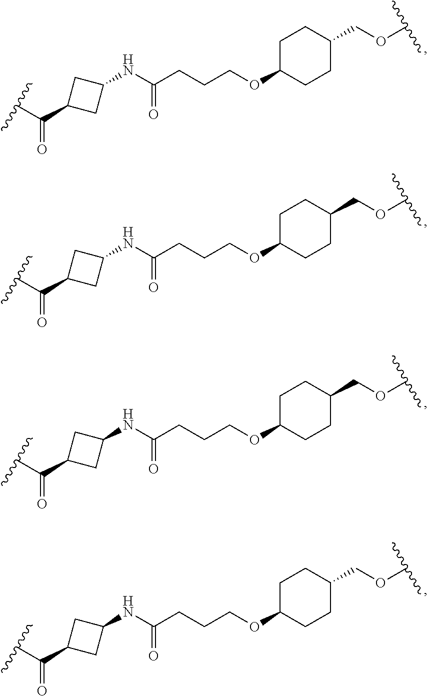

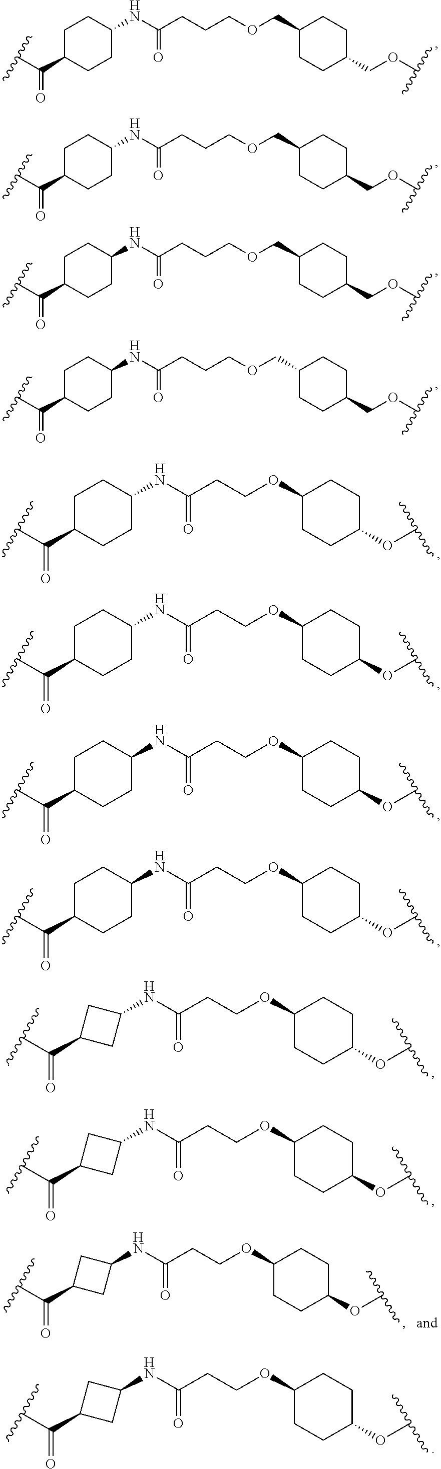

In another embodiment, L is a divalent linker of Formula (L-b):

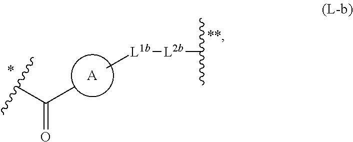

or a stereoisomer thereof,
wherein:
Ring A is C₄₋₆ cycloalkylene or C₇-9 bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is

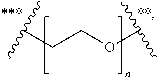

wherein n is 1, 2, 3, or 4, and

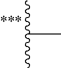

represents a covalent bond to L^{1b}; and
each R^{1b} is independently hydrogen or C₁₋₃ alkyl;
wherein

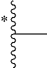

represents a covalent bond to the Y group of Formula (I), or when Y is a bond, a covalent bond to the T group of Formula (I), and

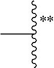

represents a covalent bond to the methylene group of Formula (I).

In another embodiment, Ring A of Formula (L-b) is

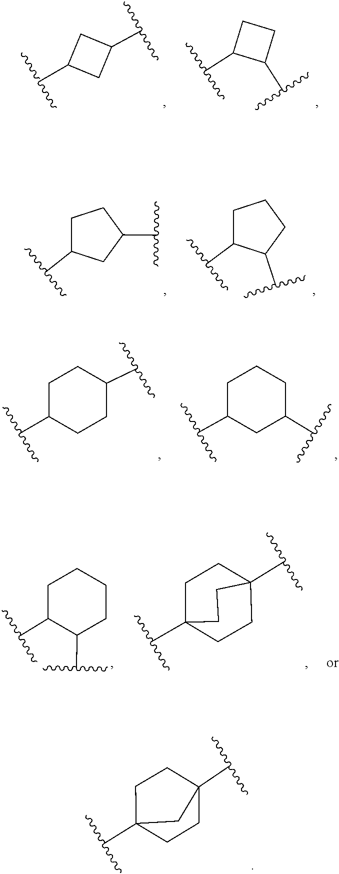

In another embodiment, L is a divalent linker of Formula (L-b-i):

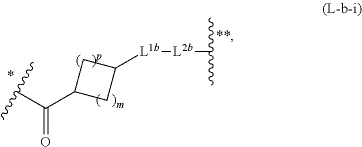

or a stereoisomer thereof,
wherein:
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is

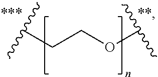

wherein n is 1, 2, 3, or 4, and

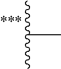

represents a covalent bond to L^{1b};
each R^{1b} is independently hydrogen or C₁₋₃ alkyl;
p is 1 or 2; and
m is 1 or 2;
wherein

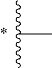

represents a covalent bond to the Y group of Formula (I), or when Y is a bond, a covalent bond to the T group of Formula (I), and

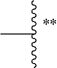

represents a covalent bond to the methylene group of Formula (I).

In another embodiment, L^{2b} of Formula (L-b) or (L-b-i) is selected from

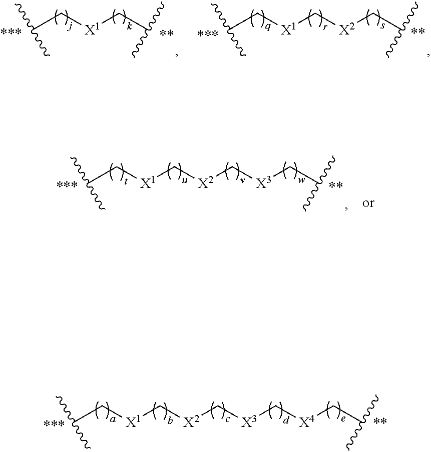

wherein:
j is 1, 2, 3, 4, 5, 6, 7, 8, 9, or 10;
k is 1, 2, 3, 4, 5, 6, 7, 8, 9, or 10;
the sum of j and k is 5, 6, 7, 8, 9, 10, or 11;
q is 1, 2, 3, 4, 5, 6, 7, 8, or 9;
r is 1, 2, 3, 4, 5, 6, 7, 8, or 9;
s is 0, 1, 2, 3, 4, 5, 6, 7, or 8;
the sum of q, r, and s is 4, 5, 6, 7, 8, 9, or 10;
t is 1, 2, 3, 4, 5, 6, or 7;
u is 1, 2, 3, 4, 5, 6, or 7;
v is 1, 2, 3, 4, 5, 6, or 7;
w is 0, 1, 2, 3, 4, 5, or 6;
the sum of t, u, v, and w is 3, 4, 5, 6, 7, 8, or 9;
a is 1, 2, 3, 4, or 5;
b is 1, 2, 3, 4, or 5;
cis 1, 2, 3, 4, or 5;
d is 1, 2, 3, 4, or 5;
e is 0, 1, 2, 3, or 4;
the sum of a, b, c, d, and e is 4, 5, 6, 7, or 8;
X¹, X², X³, and X⁴ are independently —O—, —NR^{1b}—, —C(O)NR^{1b}—, or —NR^{1b}C(O)—; and
each R^{1b} is independently hydrogen or C₁₋₃ alkyl;
wherein

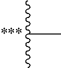

represents a covalent bond to L^{1b} of Formula (L-b) or (L-b-i), and

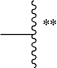

represents a covalent bond to the methylene group of Formula (I).

In another embodiment, L is a divalent linker of Formula (L-b) selected from the group

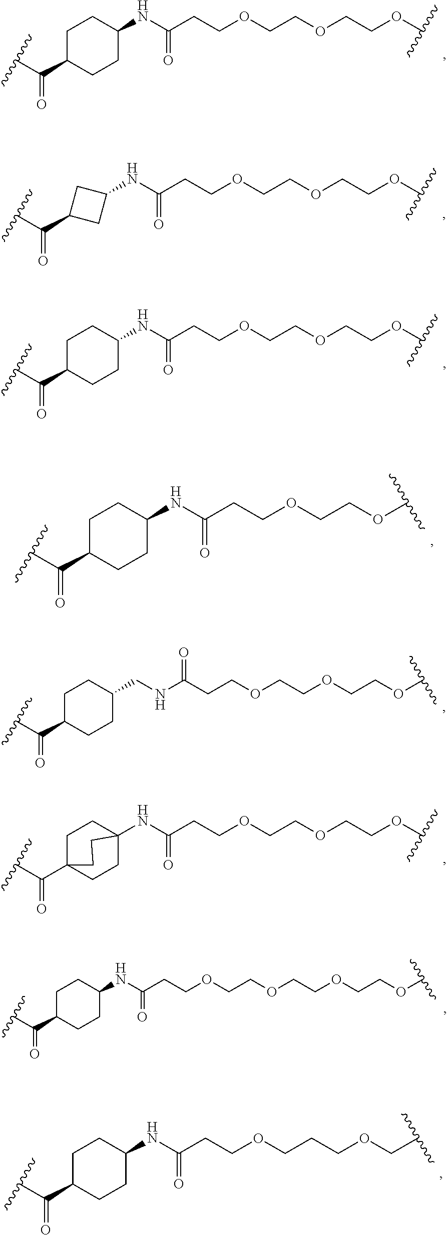

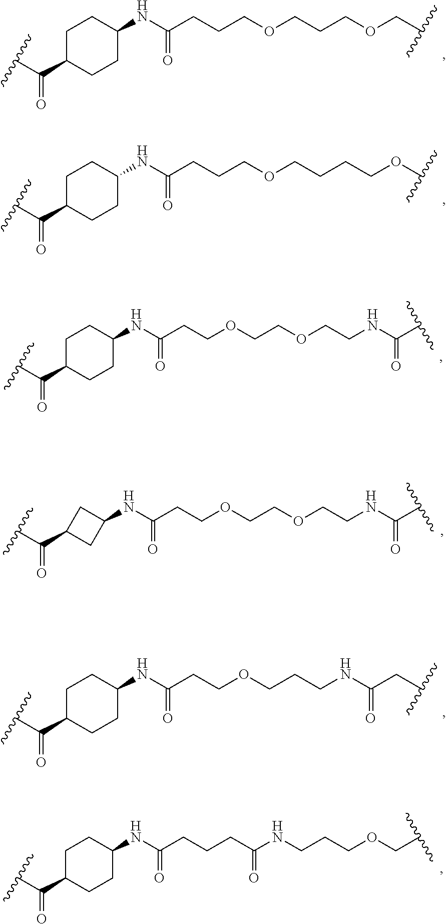 [SMILES: *COCCCOCCCC(=O)N[C@H]1CC[C@@H](C(*)=O)CC1 ; *OCCCCOCCCC(=O)N[C@H]1CC[C@H](C(*)=O)CC1 ; *C(=O)NCCOCCOCCC(=O)N[C@H]1CC[C@@H](C(*)=O)CC1 ; *C(=O)NCCOCCOCCC(=O)N[C@H]1C[C@@H](C(*)=O)C1 ; *CC(=O)NCCCOCCC(=O)N[C@H]1CC[C@@H](C(*)=O)CC1 ; *COCCCNC(=O)CCCC(=O)N[C@H]1CC[C@@H](C(*)=O)CC1]

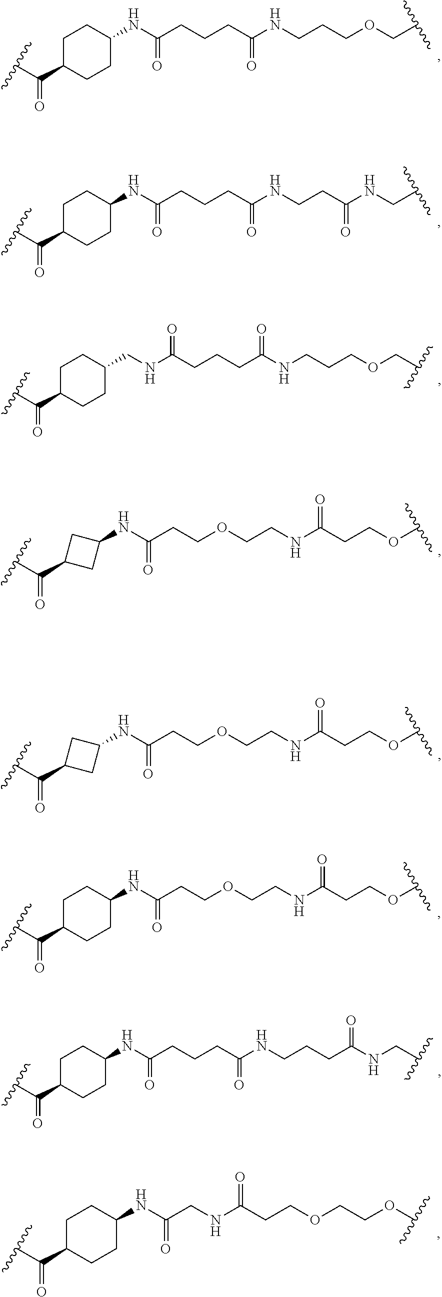

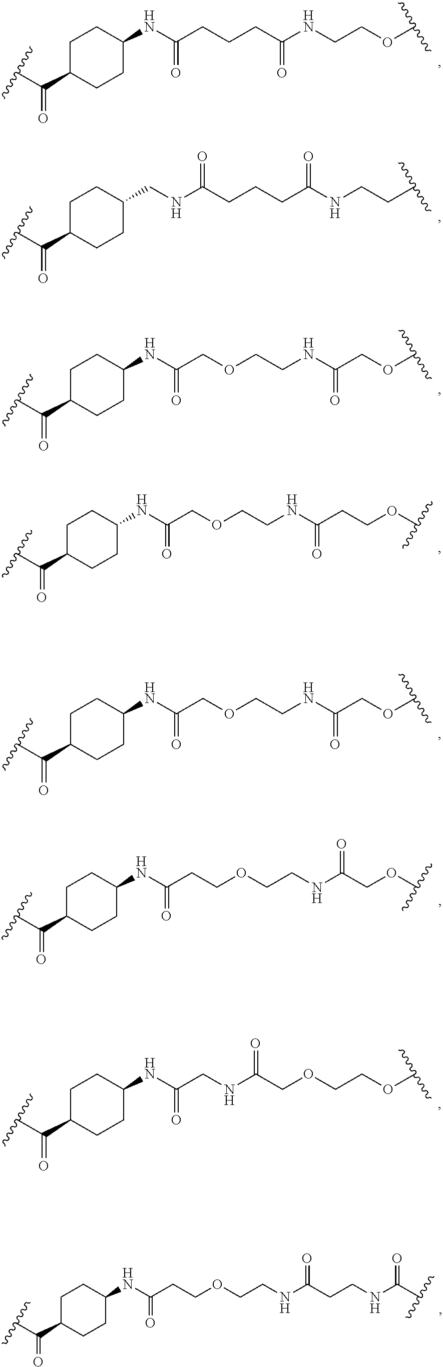

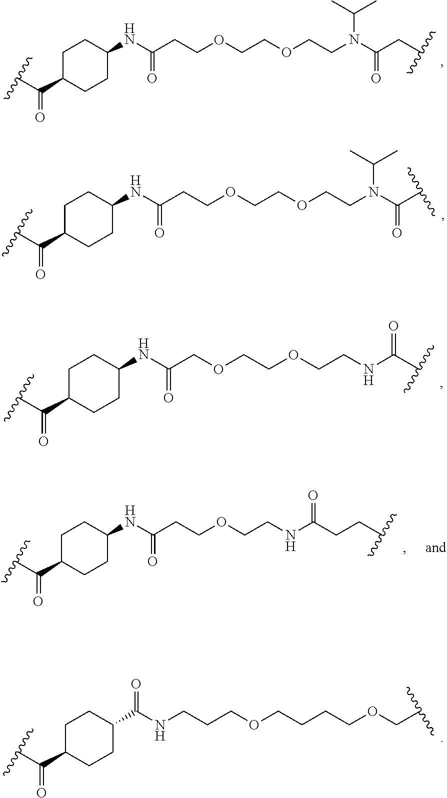 [SMILES: *CC(=O)N(CCOCCOCCC(=O)N[C@H]1CC[C@@H](C(*)=O)CC1)C(C)C ; *C(=O)N(CCOCCOCCC(=O)N[C@H]1CC[C@@H](C(*)=O)CC1)C(C)C ; *C(=O)NCCOCCOCC(=O)N[C@H]1CC[C@@H](C(*)=O)CC1 ; *CCC(=O)NCCOCCC(=O)N[C@H]1CC[C@@H](C(*)=O)CC1 ; *COCCCCOCCCNC(=O)[C@H]1CC[C@H](C(*)=O)CC1]

In another embodiment, L is a divalent linker of Formula (L-c):

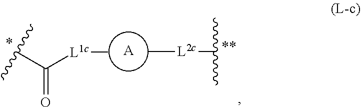

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇₋₉ bridged bicyclic cycloalkylene; and
L^{2c} is —O— or a saturated C₂₋₁₀ linear alkylene, wherein 1, 2, or 3 methylene units are replaced with —O—, —NH—, —NHC(O)—, or —C(O)NH—;
wherein

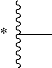

represents a covalent bond to the Y group of Formula (I), or when Y is a bond, a covalent bond to the T group of Formula (I), and

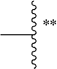

represents a covalent bond to the methylene group of Formula (I).

In another embodiment, Ring A of Formula (L-c) is

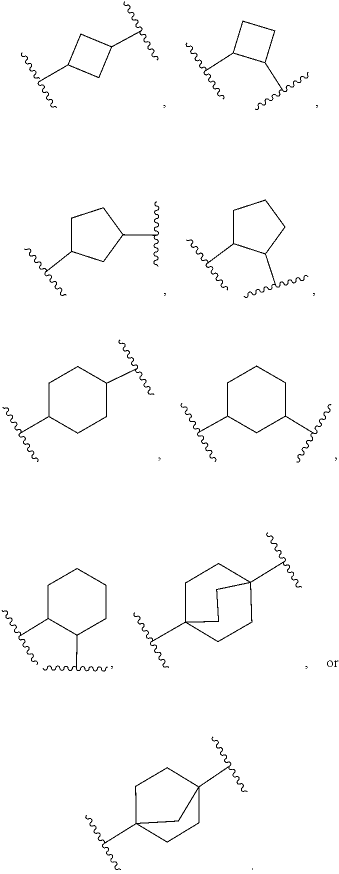

In another embodiment, L is a divalent linker of Formula (L-c-i):

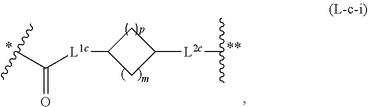

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
L²c is —O— or a saturated C₂₋₁₀ linear alkylene, wherein 1, 2, or 3 methylene units are replaced with —O—, —NH—, —NHC(O)—, or —C(O)NH—;
p is 1 or 2; and
m is 1 or 2;
wherein

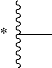

represents a covalent bond to the Y group of Formula (I), or when Y is a bond, a covalent bond to the T group of Formula (I), and

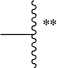

represents a covalent bond to the methylene group of Formula (I).

In another embodiment, L^{1c} of Formula (L-c) or (L-c-i) is selected from

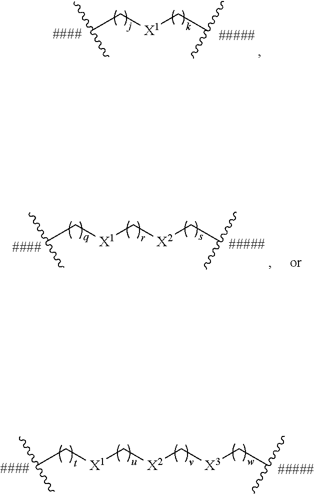

wherein:
j is 1, 2, 3, 4, 5, 6, 7, 8, or 9;
k is 0, 1, 2, 3, 4, 5, 6, 7, or 8;
the sum of j and k is 1, 2, 3, 4, 5, 6, 7, 8, or 9;
q is 1, 2, 3, 4, 5, 6, or 7;
r is 1, 2, 3, 4, 5, 6, or 7;
s is 0, 1, 2, 3, 4, 5, or 6;
the sum of q, r, and s is 2, 3, 4, 5, 6, 7, or 8;
t is 1, 2, 3, 4, or 5;
u is 1, 2, 3, 4, or 5;
v is 1, 2, 3, 4, or 5;
w is 0, 1, 2, 3, or 4;
the sum of t, u, v, and w is 3, 4, 5, 6, or 7; and
X¹, X² and X³ are independently —O—, —NH—, —NHC(O)—, or —C(O)NH—;
wherein

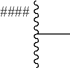

represents a covalent bond to the C(O) group of Formula (L-c) or (L-c-i), and

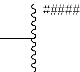

represents a covalent bond to the ring of Formula (L-c) or (L-c-i).

In another embodiment, L^{2c} of Formula (L-c) or (L-c-i) is selected from

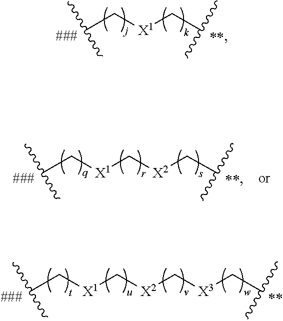

wherein:
j is 0, 1, 2, 3, 4, 5, 6, 7, 8, or 9;
k is 0, 1, 2, 3, 4, 5, 6, 7, 8, or 9;
the sum of j and k is 1, 2, 3, 4, 5, 6, 7, 8, or 9;
q is 0, 2, 3, 4, 5, 6, or 7;
r is 1, 2, 3, 4, 5, 6, 7, or 8;
s is 0, 1, 2, 3, 4, 5, 6, or 7;
the sum of q, r, and s is 1, 2, 3, 4, 5, 6, 7, or 8;
t is 0, 1, 2, 3, 4, or 5;
u is 1, 2, 3, 4, 5, or 6;
v is 1, 2, 3, 4, 5, or 6;
w is 0, 1, 2, 3, 4, or 5;
the sum of t, u, v, and w is 2, 3, 4, 5, 6, or 7; and
X¹, X² and X³ are independently —O—, —NH—, —NHC(O)—, or —C(O)NH—;
wherein

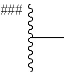

represents a covalent bond to the ring of Formula (L-c) or (L-c-i), and

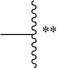

represents a covalent bond to the methylene group of Formula (I).

In another embodiment, L is a divalent linker of Formula (L-c) selected from the group consisting of:

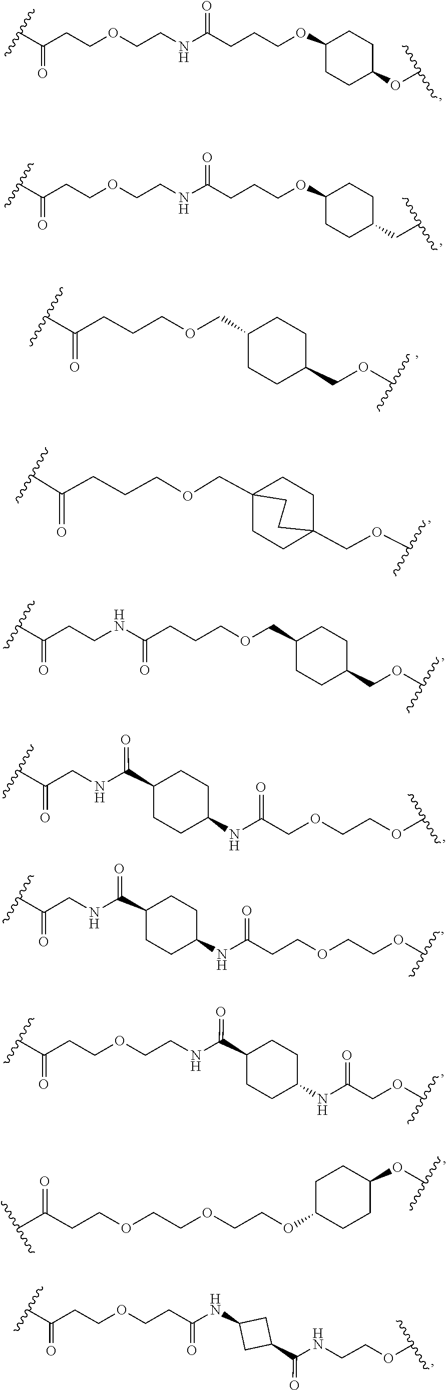

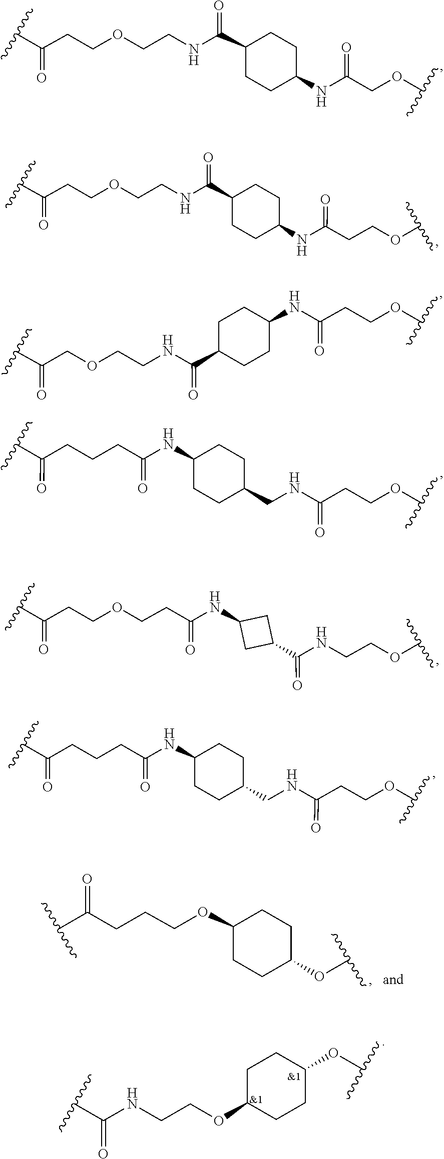

In another embodiment, L is a divalent linker of Formula (L-d):

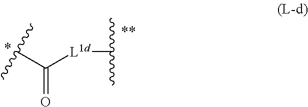

wherein:
L^{1d} is O₁₂₋₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—;
wherein

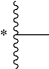

represents a covalent bond to the Y group of Formula (I), or when Y is a bond, a covalent bond to the T group of Formula (I), and

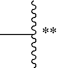

represents a covalent bond to the methylene group of Formula (I). In another embodiment, L^{1d} is a C₁₂, C₁₃, C₁₄, C₁₅, C₁₆, C₁₇, C18, C₁₉, C₂₀, C₂₁, C₂₂, C₂₃, C₂₄, C₂₅, C₂₆, C₂₇, C₂₈, C₂₉, C₃₀, or C₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—. In another embodiment, L^{1d} is O₁₂₋₂₂ linear alkylene, for example, C₁₂, C₁₃, C₁₄, C₁₅, C₁₆, C₁₇, C18, C₁₉, C₂₀, C₂₁, or C₂₂, wherein 1, 2, 3, 4, or 5 methylene units are replaced with —NH—, —O—, —C(O)NH—, —NHC(O)—, or —NHC(O)—NH—.

In another embodiment, L^{1d} of Formula (L-d) is selected from

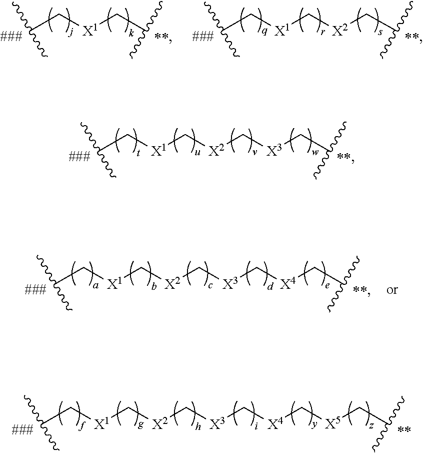

wherein:
j is 1, 2, 3, 4, 5, 6, 7, 8, 9, 10, 11, 12, 13, 14, 15, 16, 17, 18, 19, or 20;
k is 1, 2, 3, 4, 5, 6, 7, 8, 9, 10, 11, 12, 13, 14, 15, 16, 17, 18, 19, or 20;
the sum of j and k is 11, 12, 13, 14, 15, 16, 17, 18, 19, 20, or 21;
q is 1, 2, 3, 4, 5, 6, 7, 8, 9, 10, 11, 12, 13, 14, 15, 16, 17, 18, or 19;
r is 1, 2, 3, 4, 5, 6, 7, 8, 9, 10, 11, 12, 13, 14, 15, 16, 17, 18, or 19;
s is 0, 1, 2, 3, 4, 5, 6, 7, 8, 9, 10, 11, 12, 13, 14, 15, 16, 17, or 18;
the sum of q, r, and s is 10, 11, 12, 13, 14, 15, 16, 17, 18, 19, or 20;
t is 1, 2, 3, 4, 5, 6, 7, 8, 9, 10, 11, 12, 13, 14, 15, 16, or 17;
u is 1, 2, 3, 4, 5, 6, 7, 8, 9, 10, 11, 12, 13, 14, 15, 16, or 17;
v is 1, 2, 3, 4, 5, 6, 7, 8, 9, 10, 11, 12, 13, 14, 15, 16, or 17;
w is 0, 1, 2, 3, 4, 5, 6, 7, 8, 9, 10, 11, 12, 13, 14, 15, or 16;
the sum of t, u, v, and w is 9, 10, 11, 12, 13, 14, 15, 16, 17, 18, or 19;
a is 1, 2, 3, 4, 5, 6, 7, 8, 9, 10, 11, 12, 13, 14, or 15;
b is 1, 2, 3, 4, 5, 6, 7, 8, 9, 10, 11, 12, 13, 14, or 15;
c is 1, 2, 3, 4, 5, 6, 7, 8, 9, 10, 11, 12, 13, 14, or 15;
d is 1, 2, 3, 4, 5, 6, 7, 8, 9, 10, 11, 12, 13, 14, or 15;
e is 0, 1, 2, 3, 4, 5, 6, 7, 8, 9, 10, 11, 12, 13, or 14;
the sum of a, b, c, d, and e is 8, 9, 10, 11, 12, 13, 14, 15, 16, 17, or 18;
f is 1, 2, 3, 4, 5, 6, 7, 8, 9, 10, 11, 12, or 13;
g is 1, 2, 3, 4, 5, 6, 7, 8, 9, 10, 11, 12, or 13;
h is 1, 2, 3, 4, 5, 6, 7, 8, 9, 10, 11, 12, or 13;
i is 1, 2, 3, 4, 5, 6, 7, 8, 9, 10, 11, 12, or 13;
y is 1, 2, 3, 4, 5, 6, 7, 8, 9, 10, 11, 12, or 13;
z is 0, 1, 2, 3, 4, 5, 6, 7, 8, 9, 10, 11, or 12;
the sum of f, g, h, i, y, and z is 7, 8, 9, 10, 11, 12, 13, 14, 15, 16, or 17; and
X¹, X², X³, X⁴, and X⁵ are independently —NH—, —O—, —C(O)NH—, —NHC(O)—, or —NHC(O)—NH—;
wherein

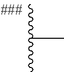

represents a covalent bond to the C(O) group of Formula (L-d), and

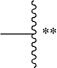

represents a covalent bond to the methylene group of Formula (I).

In another embodiment, L^{1d} of Formula (L-d) is

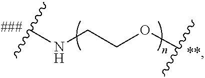

wherein n is 4, 5, 6, 7, 8, 9, or 10;
wherein

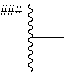

represents a covalent bond to the C(O) group of Formula (L-d), and

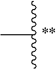

represents a covalent bond to the methylene group of Formula (I).

In another embodiment, L is a divalent linker of Formula (L-d) selected from the group consisting of:

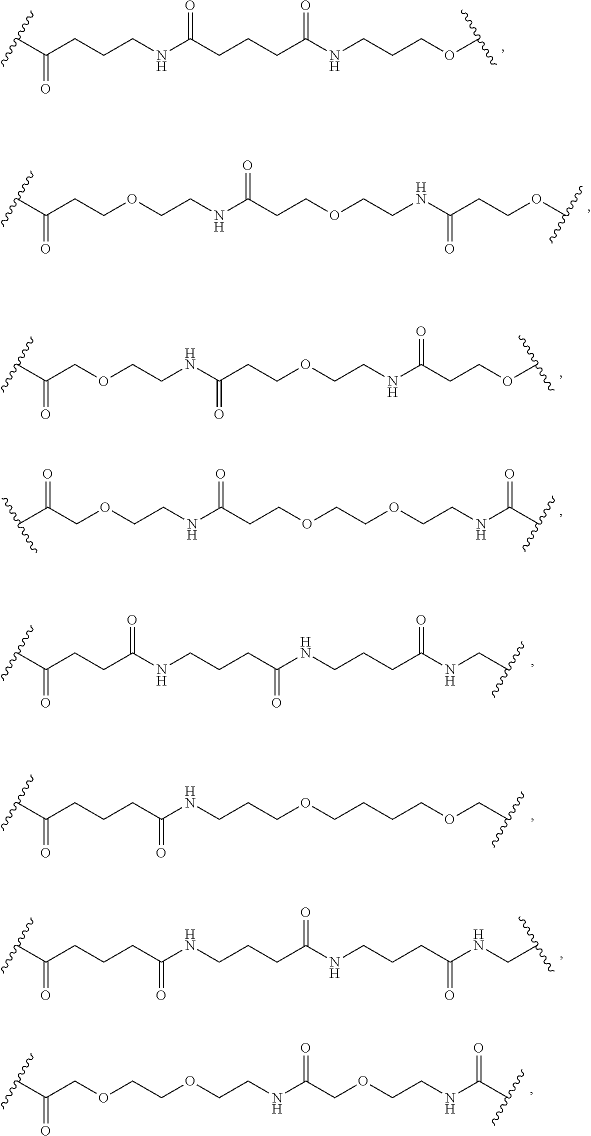

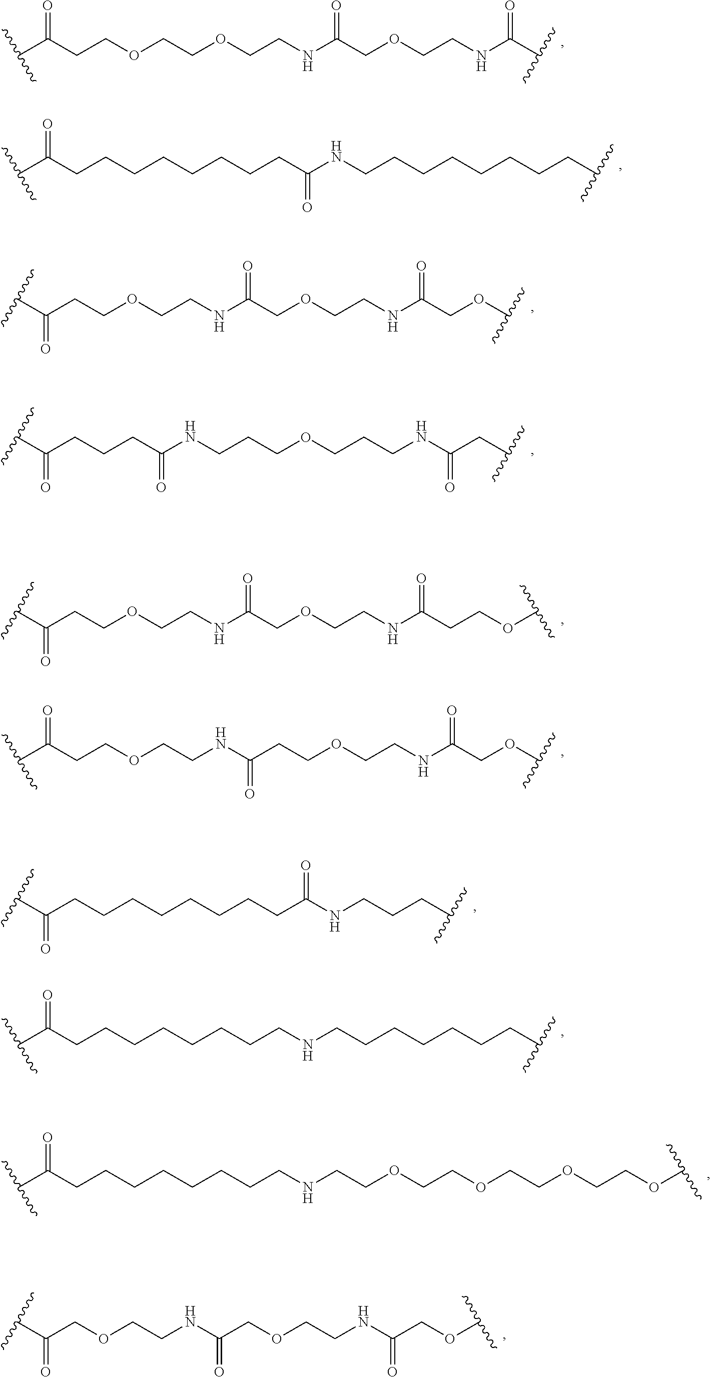

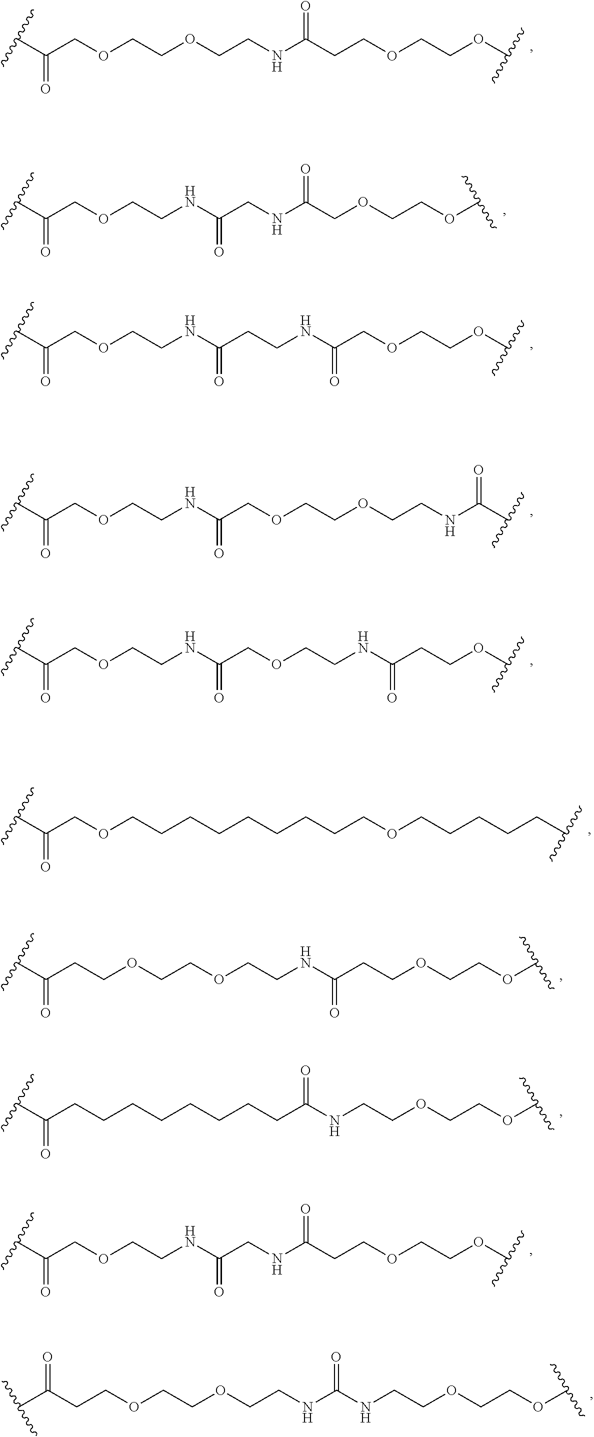

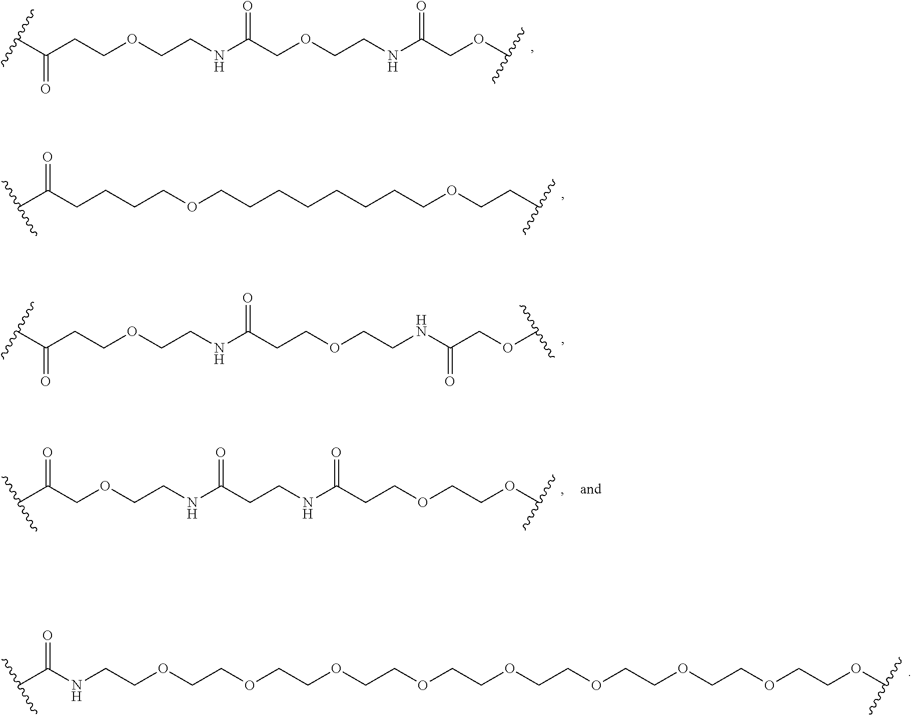 [SMILES: *OCC(=O)NCCOCC(=O)NCCOCCC(*)=O ; *CCOCCCCCCCCOCCCCC(*)=O ; *OCC(=O)NCCOCCC(=O)NCCOCCC(*)=O ; *OCCOCCC(=O)NCCC(=O)NCCOCC(*)=O ; *OCCOCCOCCOCCOCCOCCOCCOCCOCCNC(*)=O]

In another embodiment, L is a divalent linker of Formula (L-e):

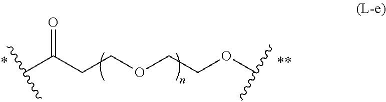

wherein:
n is an integer of 3 to 50;
wherein

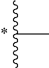

represents a covalent bond to the Y group of Formula (I), or when Y is a bond, a covalent bond to the T group of Formula (I), and

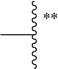

represents a covalent bond to the methylene group of Formula (I).

In another embodiment, n of Formula (L-e) is 3 to 25, 3 to 10, 3 to 8, 3 to 7, 3 to 5, or 3 to 4. In another embodiment, n of Formula (L-e) is 5 to 22, 7 to 15, or 9 to 13. In another embodiment, n of Formula (L-e) is 3, 4, 5, 7, 8, 11, 22, or 50.

In another embodiment, n of Formula (L-e) is 12 to 50, 15 to 30, 17 to 25, 18 to 24, 18 to 20, 20 to 22, or 22 to 24. In another embodiment, n of Formula (L-e) is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50. In another embodiment, n of Formula (L-e) is 19 or 23.

In another embodiment, L is a divalent linker of Formula (L-f):

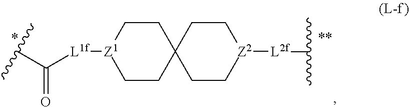

or a stereoisomer thereof,
wherein:
L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and
each of Z¹ and Z² is independently N or CH;
wherein

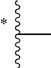

represents a covalent bond to the Y group of Formula (I), or when Y is a bond, a covalent bond to the T group of Formula (I), and

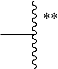

represents a covalent bond to the methylene group of Formula (I).

In another embodiment, L^{1f} of Formula (L-f) is selected from

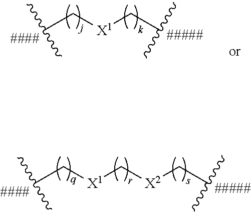

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2;
the sum of q, r, and s is 2, 3, or 4; and
X¹ and X² are independently —O—, —NH—, or —C(O)—; or —(C₃₋₆ cycloalkylene)-NHC(O)—;
wherein

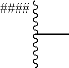

represents a covalent bond to the C(O) group of Formula (L-D, and represents a covalent bond to the ring of Formula (L-f).

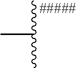

In another embodiment, L^{2f} of Formula (L-f) is selected from

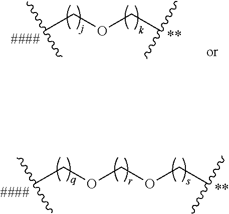

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2; and
the sum of q, r, and s is 2, 3, or 4;
wherein

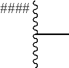

represents a covalent bond to the ring of Formula (L-f), and

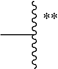

represents a covalent bond to the methylene group of Formula (I).

In another embodiment, L is a divalent linker of Formula (L-f) selected from the group consisting of:

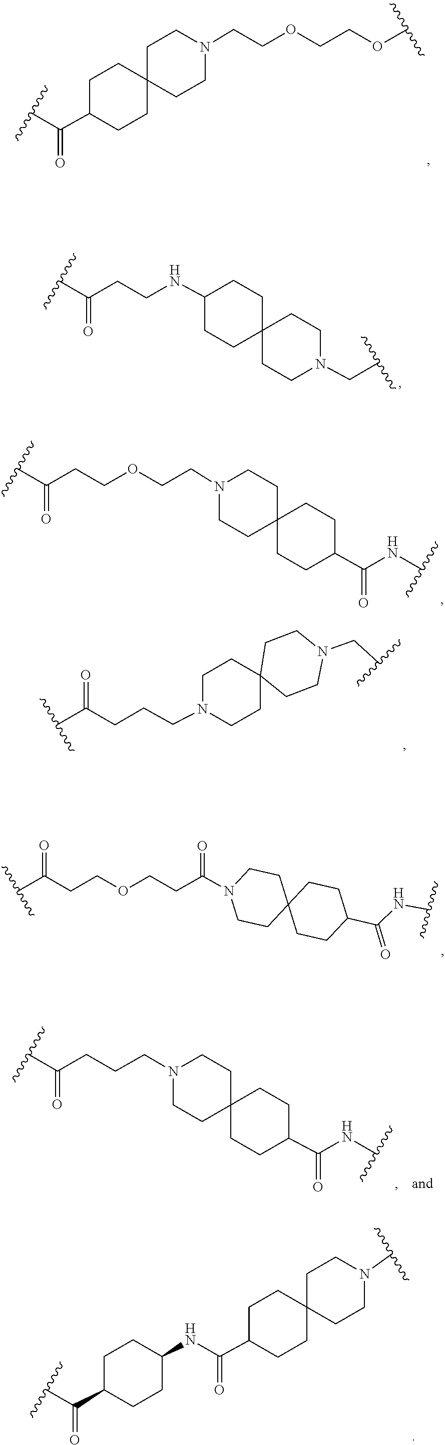

In another embodiment, L is a divalent linker of Formula (L-g):

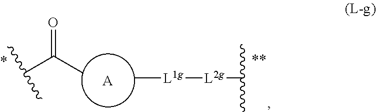

wherein:
Ring A is a 5 to 6 membered heteroarylene having 1 or 2 nitrogen ring atoms;
L^{1g} is a bond, —CH₂—, —NH—, or —O—; and
L^{2g} is

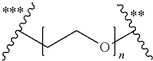

wherein n is 1, 2, 3, 4, or 5, and

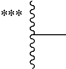

represents a covalent bond to L^{1g}; wherein

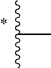

represents a covalent bond to the Y group of Formula (I), or when Y is a bond, a covalent bond to the T group of Formula (I), and

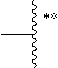

represents a covalent bond to the methylene group of Formula (I).

In another embodiment, L is a divalent linker of Formula (L-g-i):

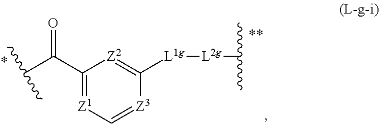

wherein:
L^{1g} is a bond, —CH₂—, —NH—, or —O—;
L²⁹ is

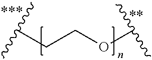

wherein n is 1, 2, 3, 4, or 5, and

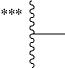

represents a covalent bond to L^{1g}; Z¹, Z², and Z³ are each independently selected from N or CH, provided that one or two of Z¹, Z², and Z³ is N;
wherein

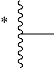

represents a covalent bond to the Y group of Formula (I), or when Y is a bond, a covalent bond to the T group of Formula (I), and

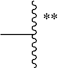

represents a covalent bond to the methylene group of Formula (I).

In another embodiment, L is a divalent linker of Formula (L-g) selected from the group consisting of:

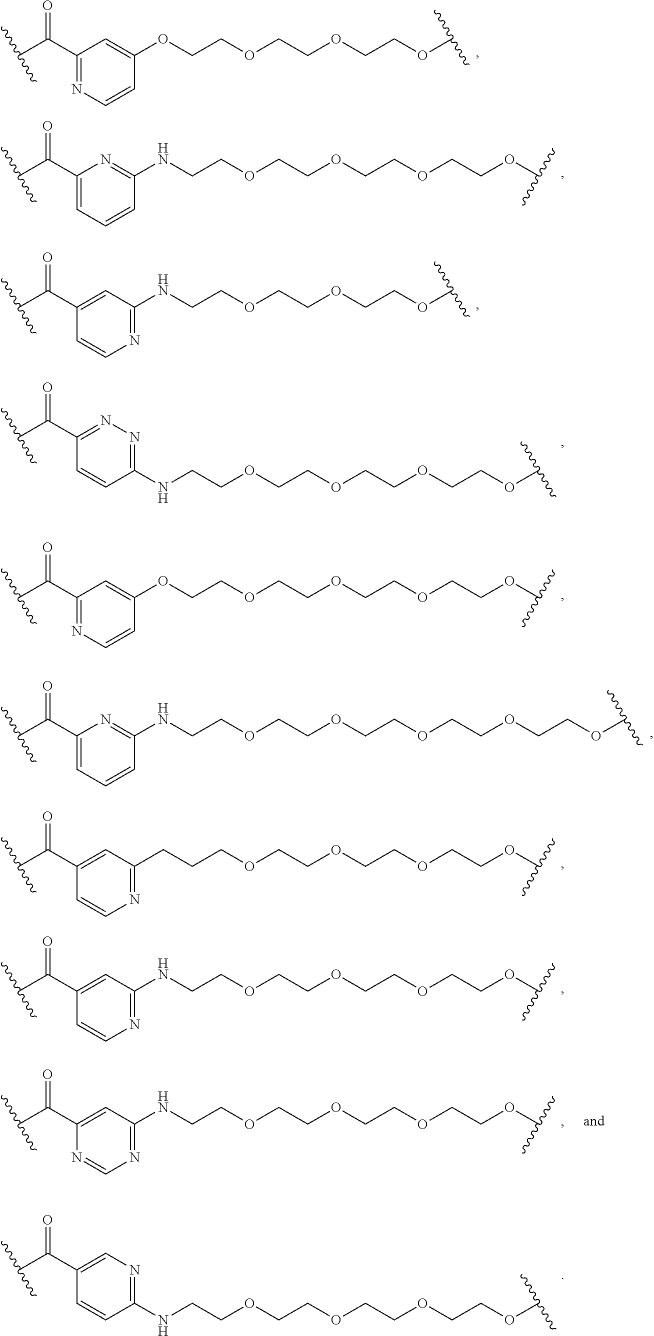

In another embodiment, L is a divalent linker of Formula (L-h):

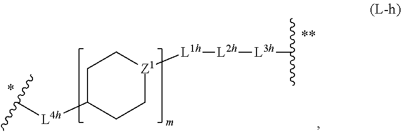

or a stereoisomer thereof,
wherein:
each Z¹ is independently N or CH;
L^{1h} is a bond, —C(O)—, —C(O)—NH—, or —NHC(O)—;
L^{2h} is C₂₋₁₀ linear alkylene or

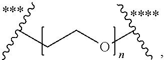

wherein n is 1, 2, 3, or 4, and

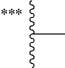

represents a covalent bond to L^{1h} and

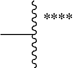

represents a covalent bond to L^{3h};
L^{3h} is a bond, —C(O)CH₂—, —O—(C₃₋₆ cycloalkylene)-O—, or —C(O)NH(CH₂)₃OCH₂—;
L^{4h} is a bond, —C(O)—, —CH₂C(O)—, or —C(O)CH₂—; and
m is 1, 2, or 3;
wherein

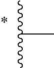

represents a covalent bond to the Y group of Formula (I), or when Y is a bond, a covalent bond to the T group of Formula (I), and

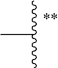

represents a covalent bond to the methylene group of Formula (I).

In another embodiment, L is a divalent linker of Formula (L-h) selected from the group consisting of:

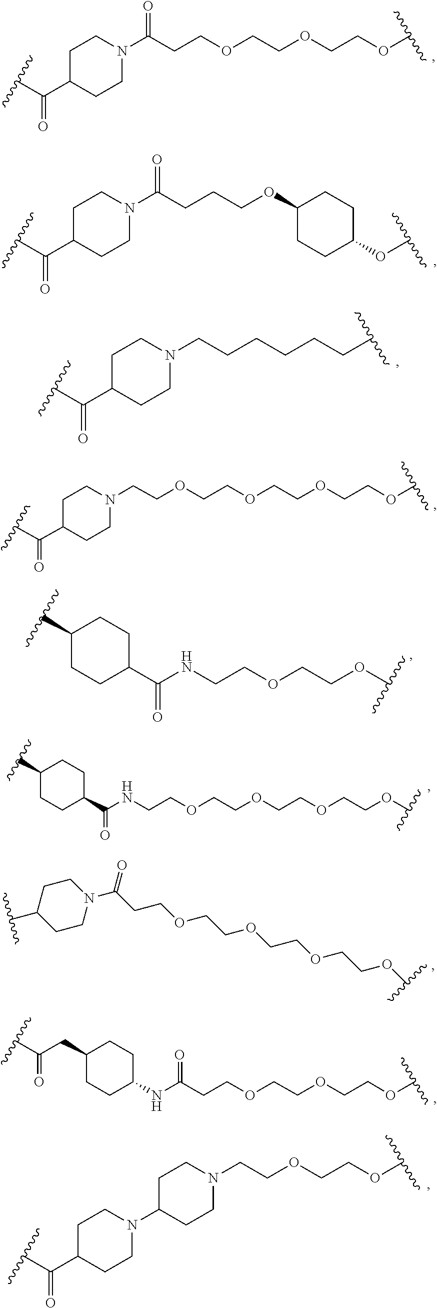

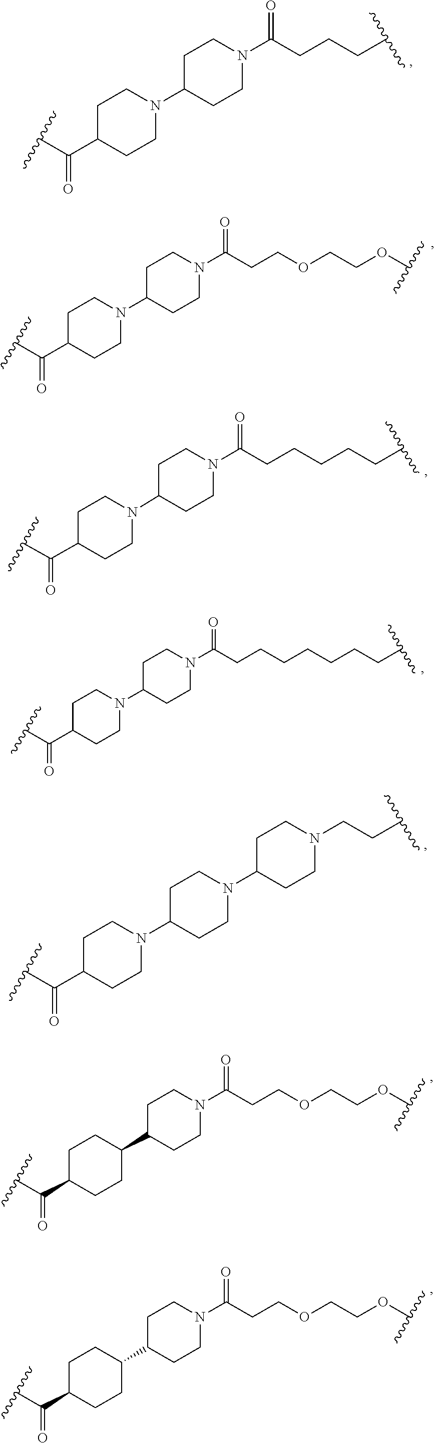

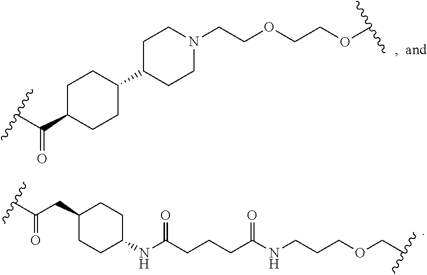 [SMILES: *OCCOCCN1CCC([C@H]2CC[C@H](C(*)=O)CC2)CC1 ; *COCCCNC(=O)CCCC(=O)N[C@H]1CC[C@H](CC(*)=O)CC1]

In another embodiment, L is a divalent linker of Formula (L-i):

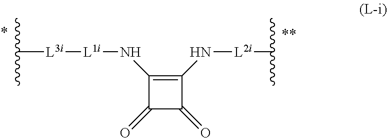

wherein:
L¹ⁱ is a bond, C₁₁₂ linear alkylene, or

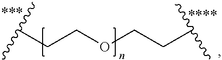

wherein n is 1, 2, 3, 4, or 5, and

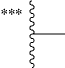

represents a covalent bond to L³ⁱ and

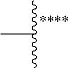

represents a covalent bond to NH;
L²ⁱ is a bond, C₁₋₁₂ linear alkylene, or

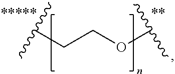

wherein n is 1, 2, 3, 4, or 5, and

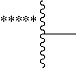

represents a covalent bond to HN; and
L³ⁱ is a bond or —C(O)—;
wherein

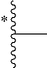

represents a covalent bond to the Y group of Formula (I), or when Y is a bond, a covalent bond to the T group of Formula (I), and

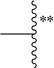

represents a covalent bond to the methylene group of Formula (I).

In another embodiment, L is a divalent linker of Formula (L-i) selected from the group

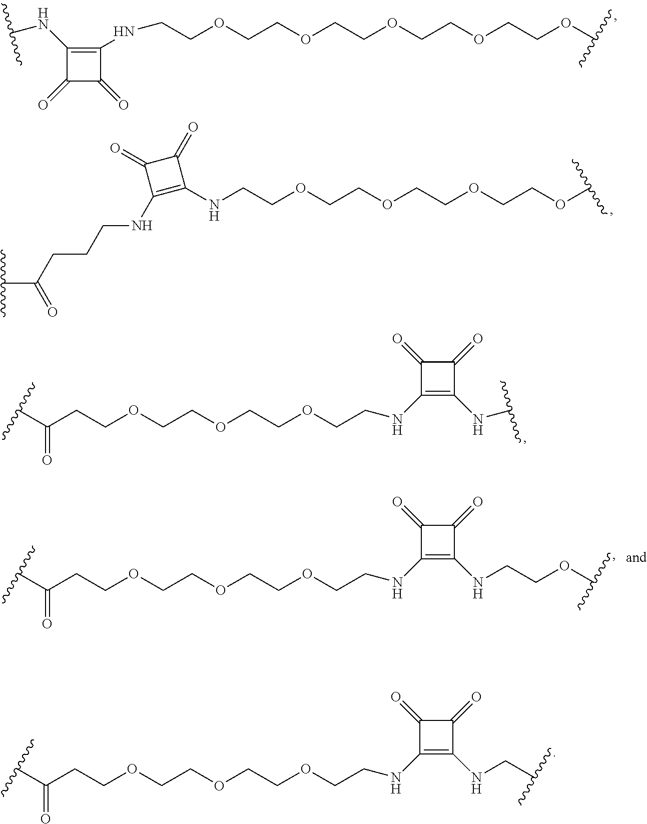

In another embodiment, L is a divalent linker of Formula (L-j):

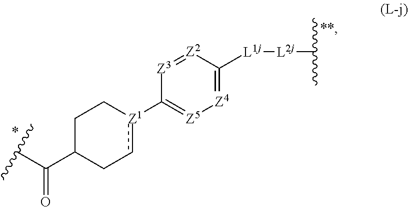

or a stereoisomer thereof,
wherein:
Z¹ is C, CH, or N;
each of Z², Z³, Z⁴ and Z⁵ is independently CH or N, provided that no more than two of Z², Z³, Z⁴
and Z⁵ are N;
L^{1j} is —NH—, —C(O)NH—, —NHC(O)—, or —O—;
L^{2j} is C₁₋₆ linear alkylene or

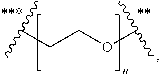

wherein n is 1 or 2, and

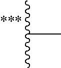

represents a covalent bond to L^{1j}; and
[symbol] represents a single bond or a double bond;
wherein

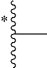

represents a covalent bond to the Y group of Formula (I), or when Y is a bond, a covalent bond to the T group of Formula (I), and

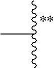

represents a covalent bond to the methylene group of Formula (I).

In another embodiment, L is a divalent linker of Formula (L-j) selected from the group consisting of:

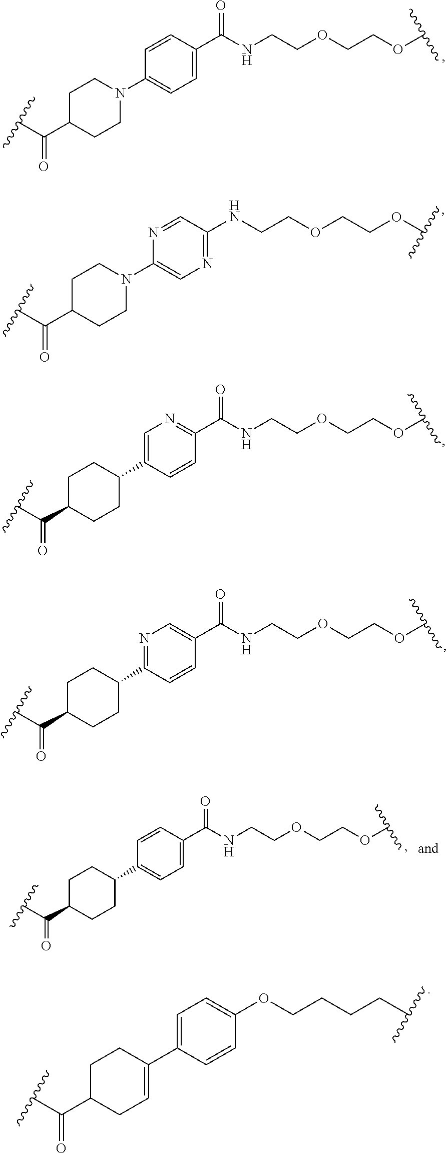 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

In another embodiment, L is a divalent linker of Formula (L-k):

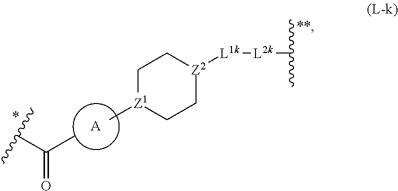

or a stereoisomer thereof,
wherein:
Ring A is phenyl or a 5 or 6 membered heteroarylene having 1 or 2 nitrogen ring atoms;
each of Z¹ and Z² is independently CH or N;
L^{1k} is a bond, —C(O)—, —C(O)NH— or —NHC(O)—; and
L^{2k} is a C₃₋₈ straight chain alkylene or

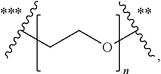

wherein n is 1, 2, or 3, and

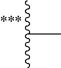

represents a covalent bond to L^{1k};
wherein

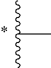

represents a covalent bond to the Y group of Formula (I), or when Y is a bond, a covalent bond to the T group of Formula (I), and

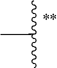

represents a covalent bond to the methylene group of Formula (I).

In another embodiment, L is a divalent linker of Formula (L-k) selected from the group consisting of:

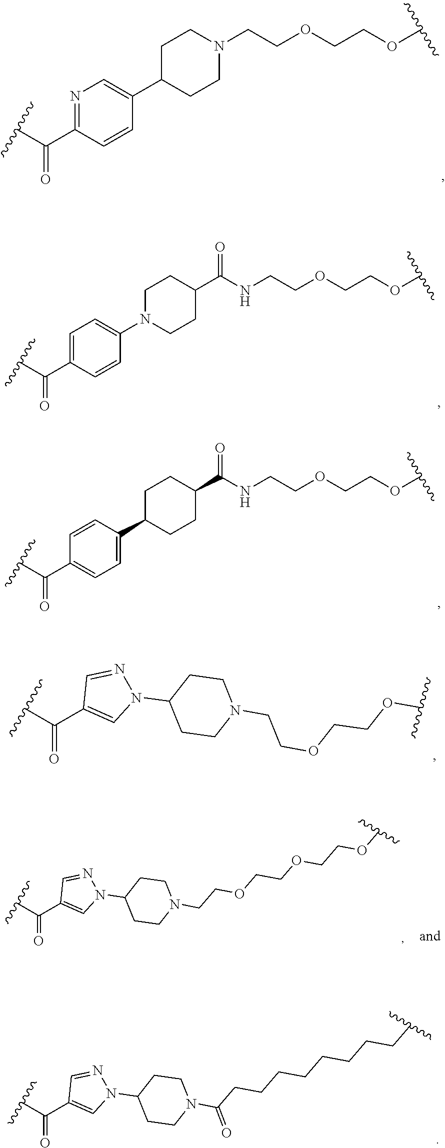 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

In another embodiment, L is a divalent linker of Formula (L-m):

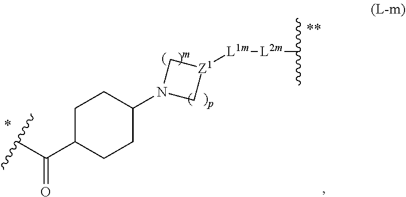

or a stereoisomer thereof,
wherein:
Z¹ is CH or N;
m is 1 or 2;
p is 1 or 2;
0, 1, or 2 hydrogen atoms of

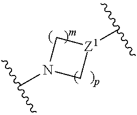

are replaced with F;
L^{1m} is a bond, —C(O)—, —C(O)NH—, —NHC(O)—, —S(O)₂NH— or —NHS(O)₂—; and
L^{2m} is C₃₋₆ linear alkylene, C₃₋₆ cycloalkylene, or

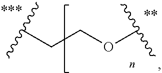

wherein n is 1 or 2, and

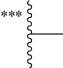

represents a covalent bond to L^{1m};
wherein

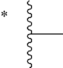

represents a covalent bond to the Y group of Formula (I), or when Y is a bond, a covalent bond to the T group of Formula (I), and

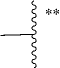

represents a covalent bond to the methylene group of Formula (I).

In another embodiment, L is a divalent linker of Formula (L-m) selected from the group

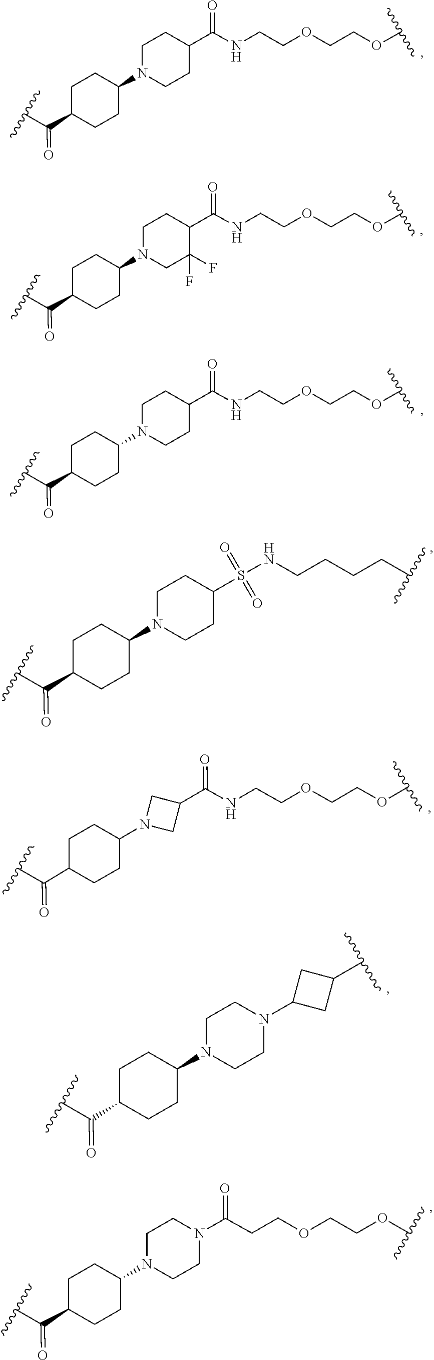

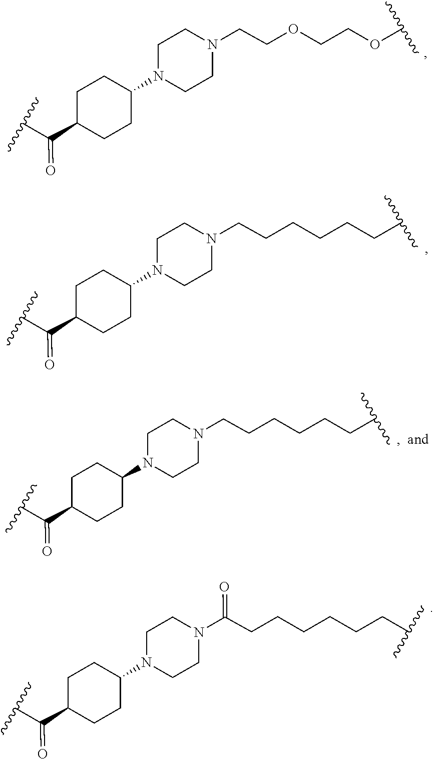 [SMILES: *OCCOCCN1CCN([C@H]2CC[C@H](C(*)=O)CC2)CC1 ; *CCCCCCN1CCN([C@H]2CC[C@H](C(*)=O)CC2)CC1 ; *CCCCCCN1CCN([C@H]2CC[C@@H](C(*)=O)CC2)CC1 ; *CCCCCCC(=O)N1CCN([C@H]2CC[C@H](C(*)=O)CC2)CC1]

In another embodiment, L is a divalent linker of Formula (L-n-i):

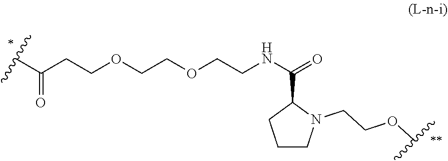 [SMILES: *OCCN1CCC[C@H]1C(=O)NCCOCCOCCC(*)=O]

wherein

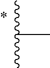

represents a covalent bond to the Y group of Formula (I), or when Y is a bond, a covalent bond to the T group of Formula (I), and

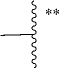

represents a covalent bond to the methylene group of Formula (I).

In another embodiment, L is a divalent linker of Formula (L-n-ii):

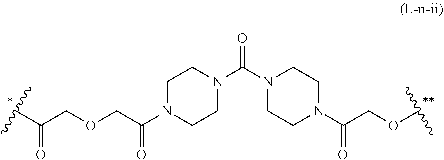 [SMILES: *OCC(=O)N1CCN(C(=O)N2CCN(C(=O)COCC(*)=O)CC2)CC1]

wherein

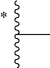

represents a covalent bond to the Y group of Formula (I), or when Y is a bond, a covalent bond to the T group of Formula (I), and

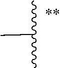

represents a covalent bond to the methylene group of Formula (I).

In another embodiment, L is a divalent linker of Formula (L-n-iii):

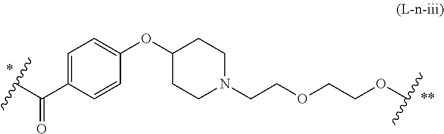 [SMILES: *OCCOCCN1CCC(Oc2ccc(C(*)=O)cc2)CC1]

wherein

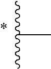

represents a covalent bond to the Y group of Formula (I), or when Y is a bond, a covalent bond to the T group of Formula (I), and

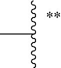

represents a covalent bond to the methylene group of Formula (I).

In another embodiment, L is a divalent linker of Formula (L-n-iv):

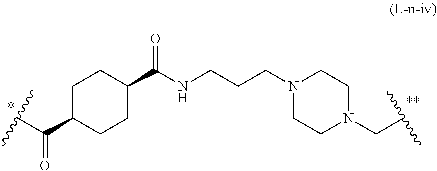 [SMILES: *CN1CCN(CCCNC(=O)[C@H]2CC[C@@H](C(*)=O)CC2)CC1]

wherein

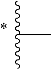

represents a covalent bond to the Y group of Formula (I), or when Y is a bond, a covalent bond to the T group of Formula (I), and

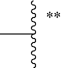

represents a covalent bond to the methylene group of Formula (I).

In another embodiment, L is a divalent linker of Formula (L-p):

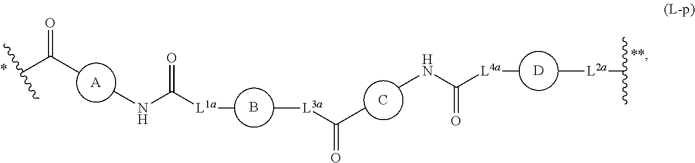

or a stereoisomer thereof,
wherein:
Ring A, Ring B, Ring C, and Ring D are each independently C₄₋₆ cycloalkylene;
L^{1a}, L^{3a}, and L^{4a} are each independently C₃₋₅ linear alkylene, wherein 1 or 2 methylene units are replaced with —O— or —NR^{a}—
each R^{a} is independently hydrogen or C₁₋₃ alkyl; and
L^{2a} is —O—, —NHC(O)—, or —CH₂—O—;
wherein

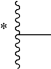

represents a covalent bond to the Y group of Formula (I), or when Y is a bond, a covalent bond to the T group of Formula (I), and

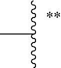

represents a covalent bond to the methylene group of Formula (I).

In another embodiment, L is a divalent linker of Formula (L-p-i):

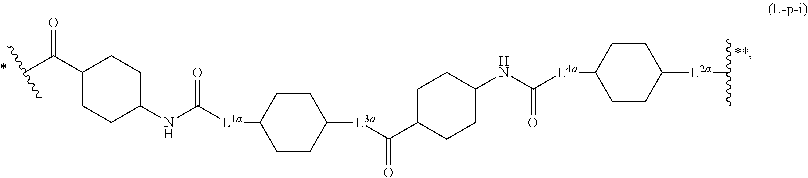

or a stereoisomer thereof,
wherein:
L^{1a}, L^{3a}, and L^{4a} are each independently C₃₋₅ linear alkylene, wherein 1 or 2 methylene units are replaced with —O— or —NR^{a}—
each R^{a} is independently hydrogen or C₁₋₃ alkyl; and
L^{2a} is —O—, —NHC(O)—, or —CH₂—O—;
wherein

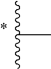

represents a covalent bond to the Y group of Formula (I), or when Y is a bond, a covalent bond to the T group of Formula (I), and

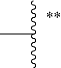

represents a covalent bond to the methylene group of Formula (I).

In another embodiment, L is a divalent linker of Formula (L-p-ii):

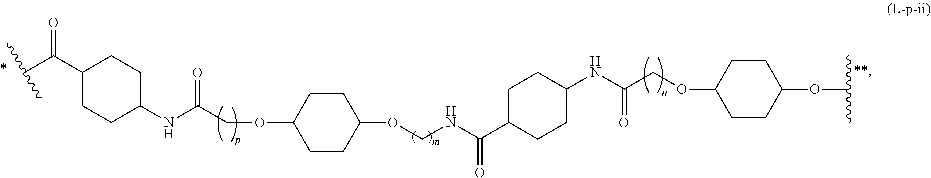

or a
stereoisomer thereof,
wherein:
p is 1, 2, or 3;
m is 1, 2, or 3; and
n is 1, 2, or 3;
wherein

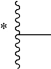

represents a covalent bond to the Y group of Formula (I), or when Y is a bond, a covalent bond to the T group of Formula (I), and

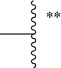

represents a covalent bond to the methylene group of Formula (I).

In another embodiment, L is a divalent linker of Formula (L-p) having the following structure:

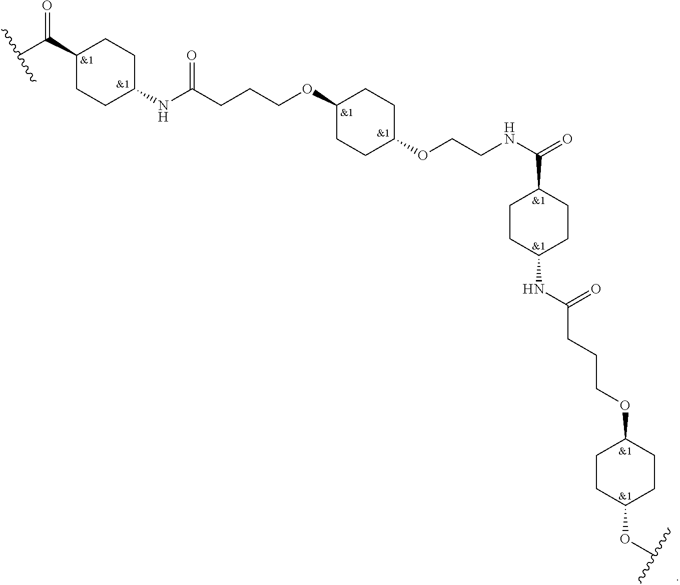 [SMILES: *O[C@H]1CC[C@H](OCCCC(=O)N[C@H]2CC[C@H](C(=O)NCCO[C@H]3CC[C@H](OCCCC(=O)N[C@H]4CC[C@H](C(*)=O)CC4)CC3)CC2)CC1]

In another embodiment, L is a divalent linker of Formula (L-q):

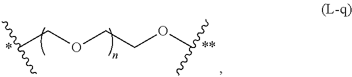

wherein
n is an integer of 10 to 30;
wherein

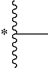

represents a covalent bond to the Y group of Formula (I), or when Y is a bond, a covalent bond to the T group of Formula (I), and

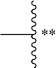

represents a covalent bond to the methylene group of Formula (I).

In another embodiment, n of Formula (L-q) is 10 to 20, 10 to 18, 12 to 16, or 13 to 15. In another embodiment, n of Formula (L-q) is 10, 11, 12, 13, 14, 15, 16, 17, 18, 19, 20, 21, 22, 23, 24, 25, 26, 27, 28, 29, or 30. In another embodiment, n of Formula (L-q) is 14.

In another embodiment, L is a divalent linker of Formula (L-r):

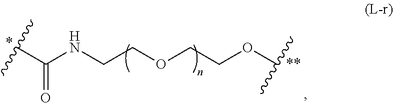

wherein
n is an integer of 10 to 30;
wherein

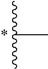

represents a covalent bond to the Y group of Formula (I), or when Y is a bond, a covalent bond to the T group of Formula (I), and

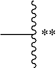

represents a covalent bond to the methylene group of Formula (I).

In another embodiment, n of Formula (L-r) is 10 to 20, 10 to 18, 12 to 16, or 13 to 15. In another embodiment, n of Formula (L-r) is 15 to 30, 17 to 28, 18 to 26, 19 to 25, 20 to 24, or 21 to 23. In another embodiment, n of Formula (L-r) is 10, 11, 12, 13, 14, 15, 16, 17, 18, 19, 20, 21, 22, 23, 24, 25, 26, 27, 28, 29, or 30. In another embodiment, n of Formula (L-r) is 14 or 22.

In one embodiment of the disclosure, Y is selected from a bond; —NH—; —(C₁₋₁₂ alkylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene.

In another embodiment, Y is selected from a bond; —NH—; —(C₁₋₆ alkylene)-O—; —O—(C₁₋₆ alkylene)-; —(C₂₋₆ alkenylene)-O—; —(C₁₋₆ alkylene)-C(O)—; —(C₂₋₆ alkenylene)-C(O)—; phenylene; piperidinylene; —(C₁₋₆ alkylene)-O-phenylene-; —(C₂₋₆ alkenylene)-O-piperidinylene; —(C₁₋₅ alkylene)-NH—, wherein 0, 1, or 2 methylene units are replaced with —O—; —NH—(C₁₋₅ alkylene)-NH—; —N(CH₃)—(C₁₋₅ alkylene)-NH—; —NH—(C₁₋₅ alkylene)-N(CH₃)—; —N(CH₃)—(C₁₋₅ alkylene)-N(CH₃)—; —(C₃₋₆ cycloalkylene)-NH—; —(C₃₋₆ cycloalkenylene)-NH—; or

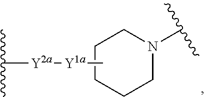

wherein Y^{1a} is a bond, —O—, —NH—, —NHC(O)—, —C(O)NH—, or C₁₋₃ alkylene; and Y^{2a} is a bond, —O—, —NH—, —NHC(O)—, —C(O)NH—, or C₁₋₃ alkylene. In another embodiment, Y is —NH—.

In another embodiment, Y is selected from the group consisting of:

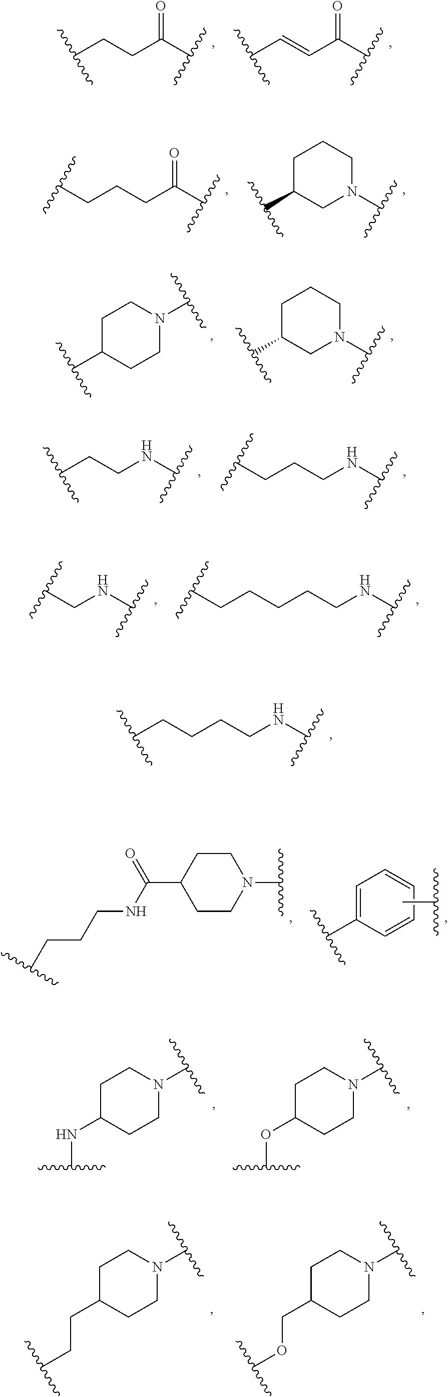

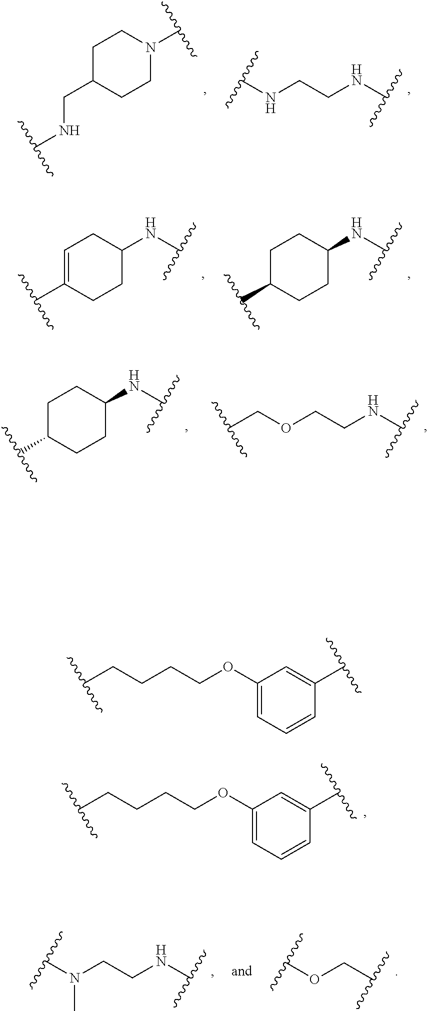

In another embodiment, R¹ is methyl, ethyl, 1-propyl, 2-propyl, 1-butyl, 2-butyl, or t-butyl. In another embodiment, R¹ is methyl. In another embodiment, R¹ is cyclopropyl, cyclobutyl, cyclopentyl, or cyclohexyl.

In another embodiment, both R² and R³ are F. In another embodiment, both R² and R³ are H. In another embodiment, one of R² and R³ is F and the other of R² and R³ is H.

In another embodiment, T is

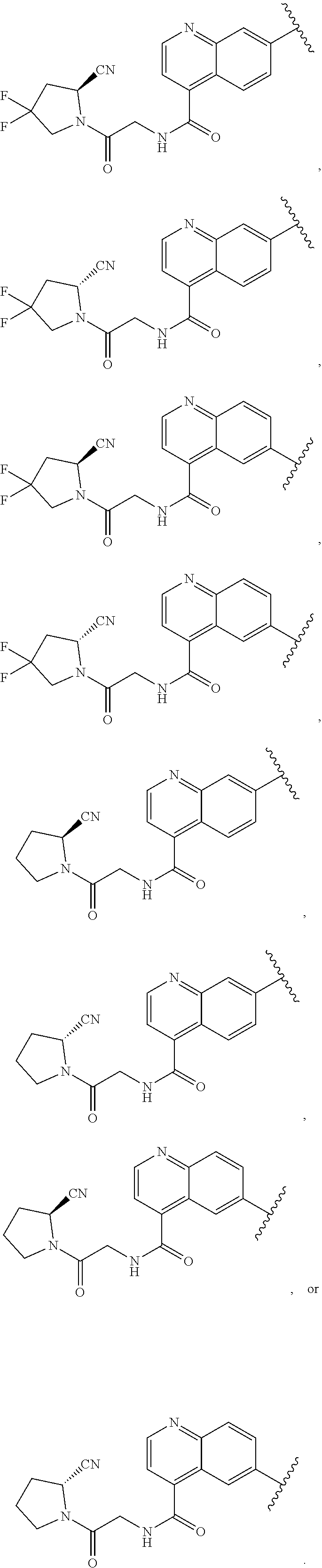

In another embodiment, the compound of Formula (I) is selected from a compound as listed in Table 1:

TABLE 1

| Ex. | |
|---|---|
| No. | Structure |
| 1 | 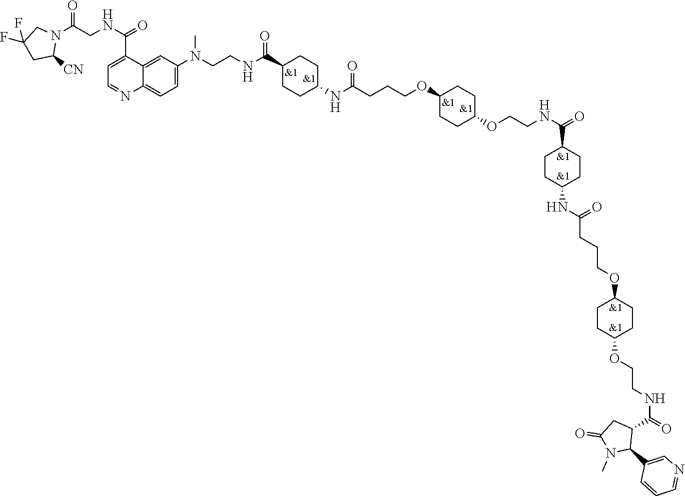 [SMILES: CN(CCNC(=O)[C@H]1CC[C@H](NC(=O)CCCO[C@H]2CC[C@H](OCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)CC1)c1ccc2nccc(C(=O)NCC(=O)N3CC(F)(F)C[C@@H]3C#N)c2c1] |
| 6 | 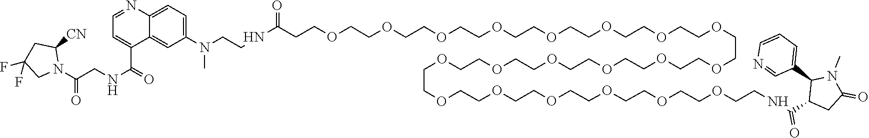 [SMILES: CN(CCNC(=O)CCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| 7 | 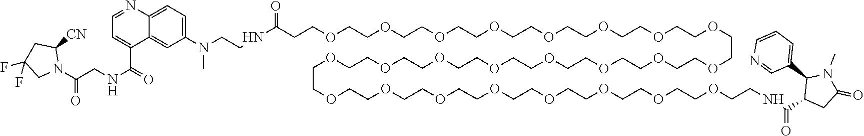 [SMILES: CN(CCNC(=O)CCOCCOCCOCCOCCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| 8 | 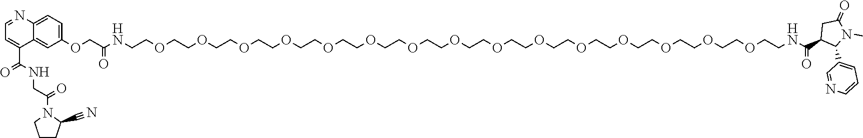 [SMILES: CN1C(=O)C[C@H](C(=O)NCCOCCOCCOCCOCCOCCOCCOCCOCCOCCOCCOCCOCCOCCOCCOCCNC(=O)COc2ccc3nccc(C(=O)NCC(=O)N4CCC[C@@H]4C#N)c3c2)[C@H]1c1cccnc1] |
| 9 | 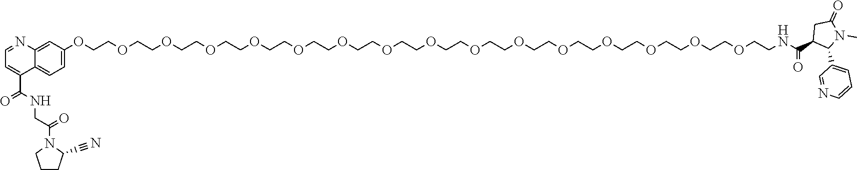 [SMILES: CN1C(=O)C[C@H](C(=O)NCCOCCOCCOCCOCCOCCOCCOCCOCCOCCOCCOCCOCCOCCOCCOCCOc2ccc3c(C(=O)NCC(=O)N4CCC[C@H]4C#N)ccnc3c2)[C@H]1c1cccnc1] |
| 10 | 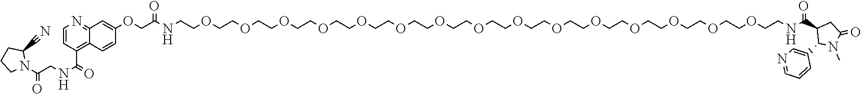 [SMILES: CN1C(=O)C[C@H](C(=O)NCCOCCOCCOCCOCCOCCOCCOCCOCCOCCOCCOCCOCCOCCOCCOCCNC(=O)COc2ccc3c(C(=O)NCC(=O)N4CCC[C@H]4C#N)ccnc3c2)[C@H]1c1cccnc1] |
| 11 | 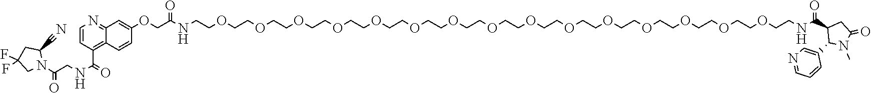 [SMILES: CN1C(=O)C[C@H](C(=O)NCCOCCOCCOCCOCCOCCOCCOCCOCCOCCOCCOCCOCCOCCOCCOCCNC(=O)COc2ccc3c(C(=O)NCC(=O)N4CC(F)(F)C[C@H]4C#N)ccnc3c2)[C@H]1c1cccnc1] |
| 12 | 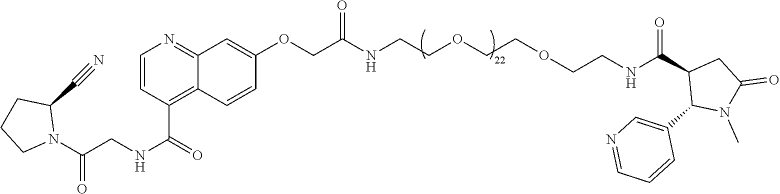 |
| 13 | 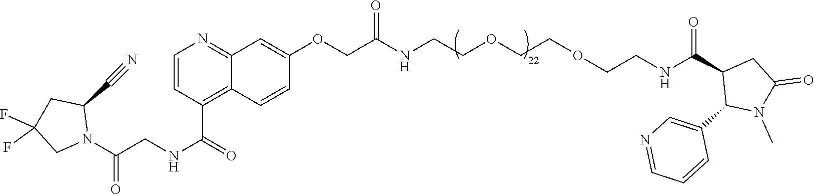 |

In one aspect, the present disclosure provides a compound of Formula (II):

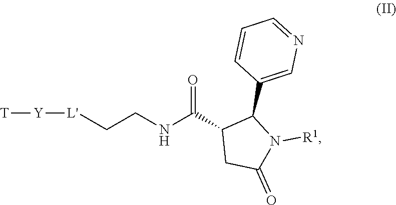

or a pharmaceutically acceptable salt thereof,
wherein:
T is

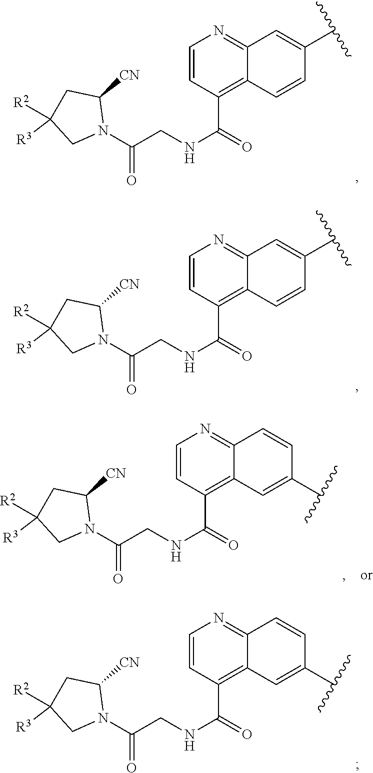

R¹ is C₁₋₄ alkyl or C₃₋₆ cycloalkyl;
R² and R³ are each independently F or H;
L′ is

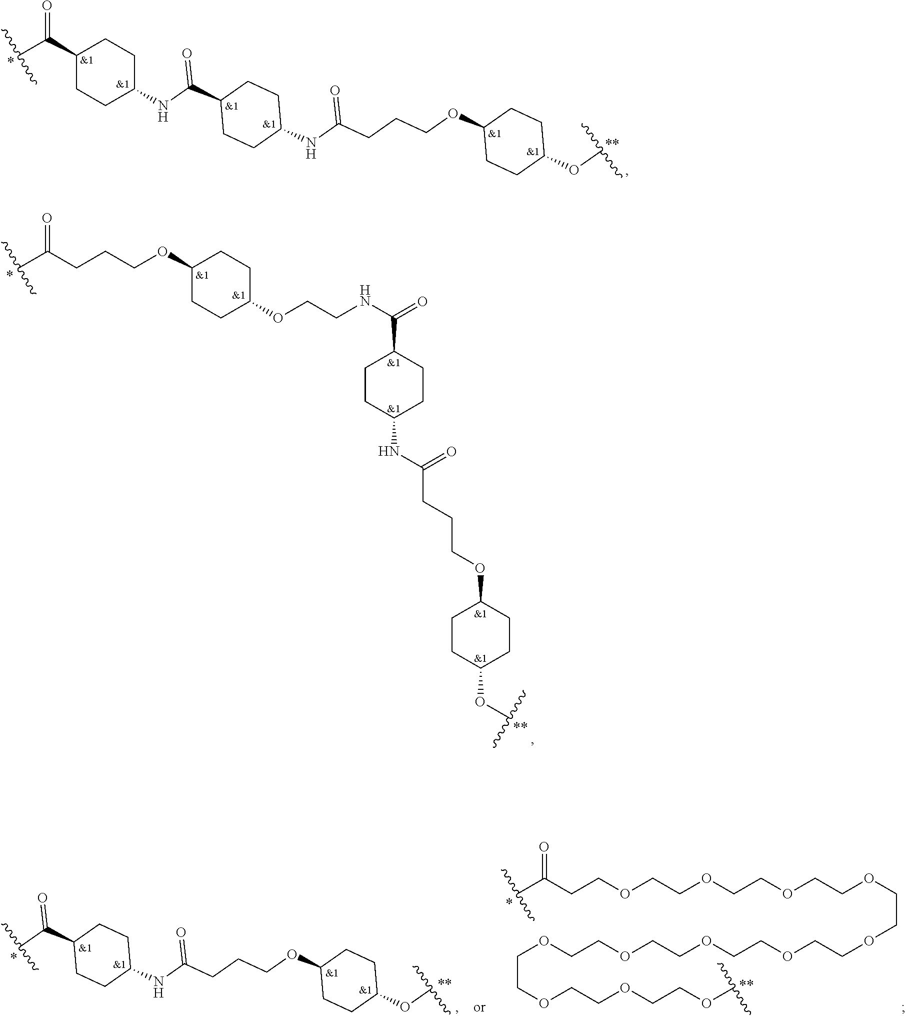 [SMILES: *O[C@H]1CC[C@H](OCCCC(=O)N[C@H]2CC[C@H](C(=O)N[C@H]3CC[C@H](C(*)=O)CC3)CC2)CC1 ; *O[C@H]1CC[C@H](OCCCC(=O)N[C@H]2CC[C@H](C(=O)NCCO[C@H]3CC[C@H](OCCCC(*)=O)CC3)CC2)CC1 ; *O[C@H]1CC[C@H](OCCCC(=O)N[C@H]2CC[C@H](C(*)=O)CC2)CC1 ; *OCCOCCOCCOCCOCCOCCOCCOCCOCCOCCOCCOCCC(*)=O]

and
Y is a bond or a divalent spacer moiety of one to twelve atoms in length;
wherein each

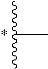

represents a covalent bond to the Y group of Formula (II), or when Y is a bond, a covalent bond to the T group of Formula (II), and each

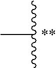

represents a covalent bond to the methylene group of Formula (II).

In one embodiment of the disclosure, Y is selected from a bond; —NH—; —(C₁₋₁₂ alkylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene.

In another embodiment, Y is selected from a bond; —NH—; —(C₁₋₆ alkylene)-O—; —O—(C₁₋₆ alkylene)-; —(C₂₋₆ alkenylene)-O—; —(C₁₋₆ alkylene)-C(O)—; —(C₂₋₆ alkenylene)-C(O)—; phenylene; piperidinylene; —(C₁₋₆ alkylene)-O-phenylene-; —(C₂₋₆ alkenylene)-O-piperidinylene; —(C₁₋₅alkylene)-NH—, wherein 0, 1, or 2 methylene units are replaced with —O—; —NH—(C₁₋₅ alkylene)-NH—; —N(CH₃)—(C₁₋₅ alkylene)-NH—; —NH—(C₁₋₅ alkylene)-N(CH₃)—; —N(CH₃)—(C₁₋₅ alkylene)-N(CH₃)—; —(C₃₋₆ cycloalkylene)-NH—; —(C₃₋₆ cycloalkenylene)-NH—; or

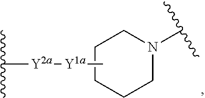

wherein Y^{1a} is a bond, —O—, —NH—, —NHC(O)—, —C(O)NH—, or C₁₋₃ alkylene; and Y^{2a} is a bond, —O—, —NH—, —NHC(O)—, —C(O)NH—, or C₁₋₃ alkylene. In another embodiment, Y is —NH—.

In another embodiment, Y is selected from the group consisting of:

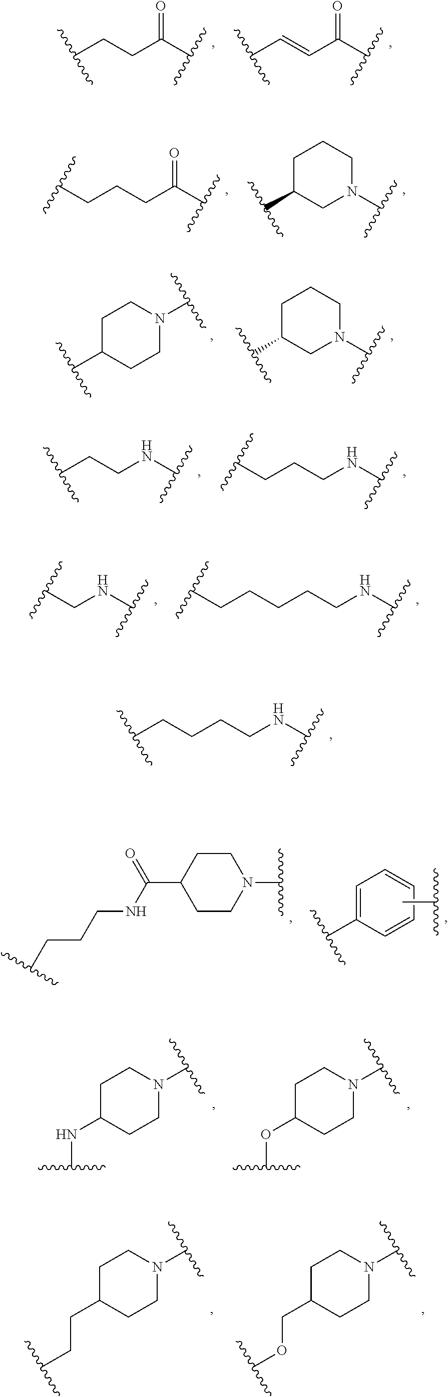

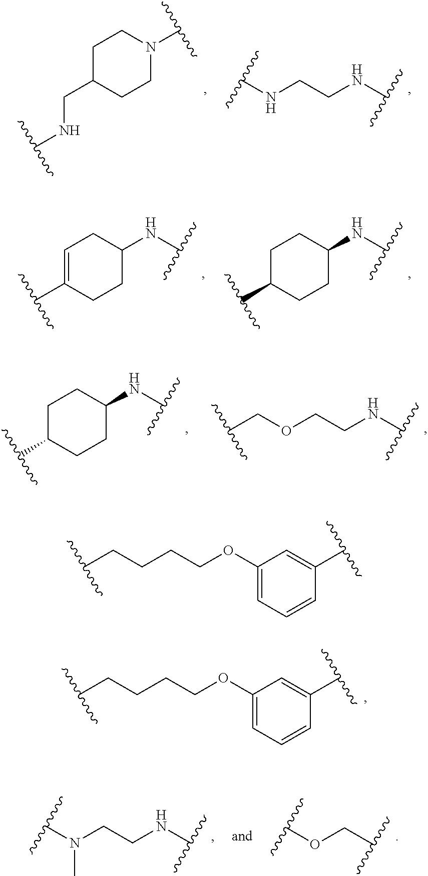

In another embodiment, R¹ is methyl, ethyl, 1-propyl, 2-propyl, 1-butyl, 2-butyl, or t-butyl.

In another embodiment, R¹ is methyl. In another embodiment, R¹ is cyclopropyl, cyclobutyl, cyclopentyl, or cyclohexyl.

In another embodiment, both R² and R³ are F. In another embodiment, both R² and R³ are H. In another embodiment, one of R² and R³ is F and the other of R² and R³ is H.

In another embodiment, T is

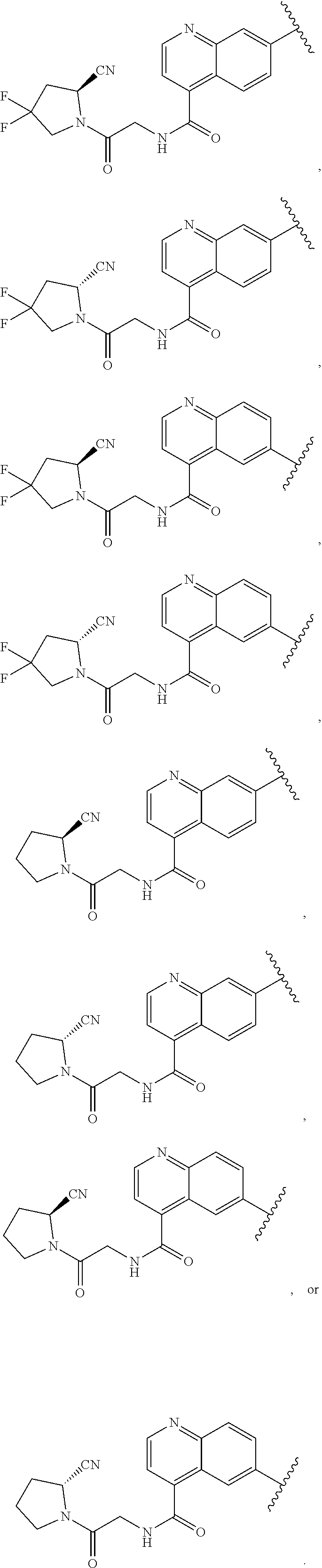

In another embodiment, the compound of Formula (II) is:

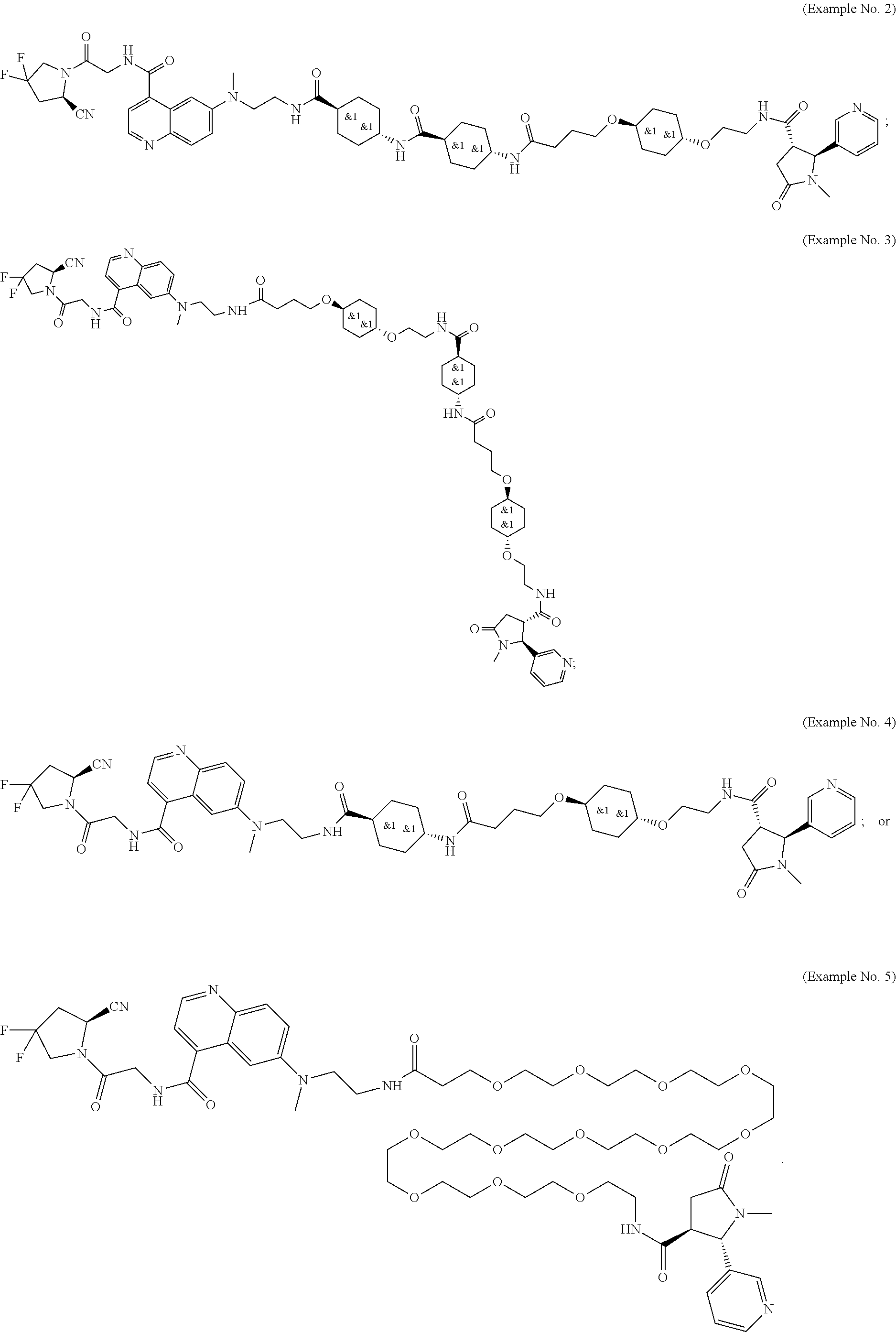

### Definitions

As used herein and in the claims, the singular forms “a” and “the” include plural reference unless the context clearly dictates otherwise.

As used herein and in the claims, the term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “pathogenic cells” includes a cell subset that causes or is capable of causing disease. Examples of pathogenic cells include, but are not limited to, cancer or tumor cells, immune cells, and stromal cells.

The term “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

The terms “effective amount” and “therapeutically effective amount” refer to an amount of a compound, or antibody, or antigen-binding portion thereof, according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The term “alkyl” represents a saturated, linear or branched hydrocarbon moiety having the specified number of carbon atoms. The term “C₁₋₃ alkyl” refers to an unsubstituted alkyl moiety containing 1, 2 or 3 carbon atoms; exemplary alkyls include methyl, ethyl and propyl.

The term “alkylene” represents a saturated, linear or branched hydrocarbon moiety having the specified number of carbon atoms, with two points of attachment. The two points of attachment can be from the same or different carbon atoms. The term “C₁₋₃ alkylene” refers to an unsubstituted alkyl moiety containing 1, 2 or 3 carbon atoms with two points of attachment; exemplary C₁₋₃ alkylene groups include methylene, ethylene and propylene.

The term “alkenyl” represents an unsaturated, linear or branched hydrocarbon moiety having the specified number of carbon atoms. The term “C₂₋₆ alkenyl” refers to an unsubstituted alkenyl moiety containing 2, 3, 4, 5, or 6 carbon atoms; exemplary alkenyls include propenyl, butenyl, pentenyl and hexenyl.

The term “alkenylene” represents an unsaturated, linear or branched hydrocarbon moiety having the specified number of carbon atoms, with two points of attachment. The two points of attachment can be from the same or different carbon atoms. The term “C₂₋₆ alkenylene” refers to an unsubstituted alkenyl moiety containing 2, 3, 4, 5, or 6 carbon atoms with two points of attachment; exemplary C₂₋₆ alkenylene groups include propenylene, butenylene, pentenylene and hexenylene.

The term “cycloalkyl” represents a saturated cyclic hydrocarbon moiety having the specified number of carbon atoms. The term “C₃₋₆ cycloalkyl” refers to an unsubstituted cycloalkyl moiety containing 3, 4, 5 or 6 carbon atoms; exemplary cycloalkyls include cyclopropyl, cyclobutyl, cyclopentyl, and cyclohexyl.

The term “cycloalkylene” represents a saturated cyclic hydrocarbon moiety having the specified number of carbon atoms, with two points of attachment. The two points of attachment can be from the same or different carbon atoms. The term “C₄₋₆ cycloalkylene” refers to an unsubstituted cycloalkylene moiety containing 4, 5, or 6 carbon atoms with two points of attachment. Exemplary cycloalkylene groups include cyclobutane-1,3-diyl, cyclopentane-1,3-diyl, cyclohexane-1,3-diyl, or cyclohexane-1,4-diyl.

The term “cycloalkenylene” represents an unsaturated cyclic hydrocarbon moiety having the specified number of carbon atoms, with two points of attachment. The two points of attachment can be from the same or different carbon atoms. The term “C₃₋₆ cycloalkenylene” refers to an unsubstituted cycloalkenylene moiety containing 3, 4, 5, or 6 carbon atoms with two points of attachment.

The term “heterocycloalkylene” refers to a saturated cyclic hydrocarbon moiety containing 1 or 2 heteroatoms independently selected from oxygen, sulphur or nitrogen atoms, with two points of attachment. The two points of attachment can be from the same or different carbon atoms. The term “3- to 6-membered heterocycloalkylene” refers to a 3- to 6-membered saturated cyclic moiety containing 2, 3, 4 or 5 carbon atoms in addition to 1 or 2 oxygen, sulphur or nitrogen atoms, with two points of attachment. Suitably, the 3- to 6-membered heterocycloalkylene group contains 1 oxygen or nitrogen atom. Suitably such group contains 3 carbon atoms and 1 oxygen or nitrogen atom, such as azetidindiyl or oxetandiyl. Suitably such group contains 4 or 5 carbon atoms and 1 oxygen or nitrogen atom, such as tetrahydrofurandiyl, tetrahydropyrandiyl, pyrrolidindiyl or piperidindiyl.

The term “bridged bicyclic cycloalkylene” refers to a saturated bicyclic hydrocarbon moiety having at least one bridge, with two points of attachment. A “bridge” is an unbranched chain of atoms or an atom or a valence bond connecting two bridgeheads, where a “bridgehead” is any skeletal atom of the ring system which is bonded to three or more skeletal atoms (excluding hydrogen). The two points of attachment can be from the same or different carbon atoms. The term “C₇₋₉ bridged bicyclic cycloalkylene” refers to an unsubstituted bridged bicyclic cycloalkylene moiety containing 7, 8, or 9 carbon atoms with two points of attachment.

The term “arylene” refers to a monocyclic or bicyclic ring system wherein at least one ring in the system is aromatic, with two points of attachment. Exemplary arylene groups include phenylene, biphenylene, naphthylene, and anthracylene.

The term “heteroarylene” refers to a monocyclic or bicyclic ring system wherein at least one ring in the system is aromatic, and having, in addition to carbon atoms, from one to five heteroatoms independently selected from oxygen, sulphur or nitrogen atoms, with two points of attachment. The term “5- to 6-membered heteroarylene” refers to a 5- to 6-membered cyclic aromatic moiety containing 2, 3, 4 or 5 carbon atoms in addition to 1, 2, or 3 heteroatoms independently selected from oxygen, sulphur or nitrogen atoms, with two points of attachment.

The skilled artisan will appreciate that salts, including pharmaceutically acceptable salts, of the compounds according to Formulae (I) and (II) may be prepared. Indeed, in certain embodiments of the invention, salts including pharmaceutically-acceptable salts of the compounds according to Formulae (I) and (II) may be preferred over the respective free or unsalted compound. Accordingly, the invention is further directed to salts, including pharmaceutically-acceptable salts, of the compounds according to Formulae (I) and (II). The invention is further directed to free or unsalted compounds of Formulae (I) and (II).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, trifluoroacetate, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), b/s-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formulae (I) and (II) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formulae (I) or (II), or in any chemical structure illustrated herein, if not specified the structure is intended to encompass all individual stereoisomers and all mixtures thereof. Thus, compounds according to Formulae (I) and (II) containing one or more chiral centers may be used as racemic mixtures, enantiomerically enriched mixtures, or as enantiomerically pure individual stereoisomers.

A mixture of stereoisomers in which the relative configuration of all of the stereocenters is known may be depicted using the symbol “&” together with an index number (e.g., “&1”). For example, a group of two stereogenic centers labeled with the symbol “&1” represents a mixture of two possible stereoisomers in which the two stereogenic centers have a relative configuration as depicted.

Divalent groups are groups having two points of attachment. For all divalent groups, unless otherwise specified, the orientation of the group is implied by the direction in which the formula or structure of the group is written.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range.

Concentrations described herein are determined at ambient temperature and pressure. This may be, for example, the temperature and pressure at room temperature or in a particular portion of a process stream. Preferably, concentrations are determined at a standard state of 25° C. and 1 bar of pressure.

### FAP Target and FAP-Binding Moieties

The compounds of Formulae (I) and (II) as disclosed herein are heterobifunctional synthetic agents designed such that one terminus interacts with a cell surface FAP target, while the other terminus binds a specific antibody. More specifically, the ARM simultaneously binds the cell surface FAP target as well as the specific antibody. This ternary complex directs immune surveillance to FAP-expressing tissue/cells and unites the mechanisms of antibody function with the dose-control of small molecules. This mechanism may include antibody dependent cellular cytotoxicity (ADCC), antibody dependent cellular phagocytosis (ADCP), or complement dependant cytotoxicity (CDC), and preferably includes ADCC. The same Fc receptor expressing immune cells that initiate destruction of the ARM/antibody tagged cells also participate in presentation of endogenous antigens for the potential for long term cellular immunity.

The compounds of Formulae (I) and (II) as disclosed herein include a FAP-binding moiety that is capable of binding FAP present on the surface of a cell. In one embodiment, the FAP is expressed on a pathogenic cell.

In a further embodiment, the pathogenic cell is a tumor cell or cancer cell, an immune cell, or a stromal cell (including stromal cells present in a tumor microenvironment).

In a further embodiment, the tumor cells or cancer cells are solid tumor cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, bladder cancer cells, bone cancer cells, esophogeal cancer cells, gastric cancer cells, renal cancer cells, melanoma cancer cells, thyroid cancer cells, or breast cancer cells, preferably pancreatic cancer cells or CRC cells.

In a further embodiment, the immune cells are macrophages.

In a further embodiment, the stromal cells are cancer associated fibroblasts (CAFs).

The present disclosure also provides a pharmaceutical composition comprising a compound of Formulae (I) or (II) as disclosed herein, and a pharmaceutically acceptable excipient, carrier, or diluent.

### Anti-Cotinine Antibodies

The present disclosure provides an antibody, or antigen-binding fragment thereof, that binds to a cotinine moiety. As used herein, the term “anti-cotinine antibody or antigen-binding fragment thereof” refers to an antibody, or antigen binding fragment thereof that binds to a cotinine moiety. Cotinine has the following structure:

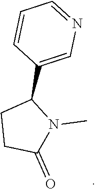

As used herein, the term “cotinine moiety” refers to cotinine or an analog of cotinine. Compounds of Formulae (I) and (II) described herein comprise a cotinine moiety linked via a linker to a FAP-binding moiety. In one embodiment, the cotinine moiety has the following structure:

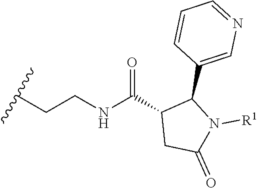

wherein R¹ is C₁₋₄ alkyl or C₃₋₆ cycloalkyl. In another embodiment, R¹ is methyl, ethyl, 1-propyl, 2-propyl, 1-butyl, 2-butyl, or t-butyl. In another embodiment, R¹ is methyl. In another embodiment, R¹ is cyclopropyl, cyclobutyl, cyclopentyl, or cyclohexyl.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, 23(9): 1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding Clq, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or A light chain. The majority of antibodies in the serum belong to the IgG class, there are four isotypes of human IgG (IgG1, IgG2, IgG3 and IgG4), the sequences of which differ mainly in their hinge region.

“CDRs” are defined as the complementarity determining region amino acid sequences of an antibody or antigen binding fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature, 1989, 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 2 below represents one definition using each numbering convention for each CDR or binding unit. It should be noted that some of the CDR definitions may vary depending on the individual publication used.

TABLE 2

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

In a further embodiment, the anti-cotinine antibody is humanized. In a further embodiment, the Fc region of the anti-cotinine antibody is modified to increase ADCC activity, ADCP activity, and/or CDC activity, suitable modifications of which are provided below. In a further embodiment, the Fc region of the anti-cotinine antibody is modified to increase ADCC activity.

Fc engineering methods can be applied to modify the functional or pharmacokinetics properties of an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function. The in vivo half-life of an antibody can be altered by making mutations that affect binding of the Fc to the FcRn (neonatal Fc receptor).

The term “effector function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antibody, or antigen binding fragment thereof, of the present invention can be assessed for ADCC effector function in a Natural Killer cell assay. Examples of such assays can be found in Shields et al., The Journal of Biological Chemistry, 2001, 276: 6591-6604; Chappel et al., The Journal of Biological Chemistry, 1993, 268: 25124-25131; Lazar et al., PNAS, 2006, 103: 4005-4010.

Examples of assays to determine CDC function include those described in J Imm Meth, 1995, 184: 29-38.

The effects of mutations on effector functions (e.g., FcRn binding, FcγRs and C1q binding, CDC, ADCML, ADCC, ADCP) can be assessed, e.g., as described in Grevys et al., J Immunol., 2015,194(11): 5497-5508; Tam et al., Antibodies, 2017, 6(3): 12; or Monnet et al., mAbs, 2014, 6(2): 422-436.

Throughout this specification, amino acid residues in Fc regions, in antibody sequences or full-length antigen binding protein sequences, are numbered according to the EU index numbering convention.

Human IgG1 constant regions containing specific mutations have been shown to enhance binding to Fc receptors. In some cases these mutations have also been shown to enhance effector functions, such as ADCC and CDC, as described below. Antibodies, or antigen binding fragments thereof, of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science, 2014, 343: 1260-1293) and E345R/E430G/S440Y results in preformed IgG hexamers (Wang et al., Protein Cell, 2018, 9(1): 63-73).

Enhanced ADCC: Fc engineering can be used to enhance ADCC. For example (with reference to IgG1), F243L/R292P/Y300L/V3051/P396L; S239D/1332E; and S298A/E333A/K334A increase FcγRIIIa binding; S239D/1332E/A330L increases FcγRIIIa binding and decreases FcγRIIb binding; G236A/S239D/1332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., mAbs, 2013, 5(2): 229-236).

Enhanced ADCP: Fc engineering can be used to enhance ADCP. For example (with reference to IgG1), G236A/S239D/1332E increases FcγRIIa binding and increases FcγRIIIa binding (Richards, J. et al., Mol. Cancer Ther., 2008, 7: 2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding Wang et al., Protein Cell, 2018, 9(1): 63-73).

In a further embodiment, an antibody, or antigen binding fragment thereof, of the present invention may comprise a heavy chain constant region with an altered glycosylation profile, such that the antibody, or antigen binding fragment thereof, has an enhanced effector function, e.g., enhanced ADCC, enhanced CDC, or both enhanced ADCC and CDC. Examples of suitable methodologies to produce an antibody, or antigen binding fragment thereof, with an altered glycosylation profile are described in WO 2003/011878, WO 2006/014679 and EP1229125.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al., J Biol Chem., 2002, 277(30): 26733-40 and Monnet et al., mAbs, 2014, 6(2): 422-436).

In one embodiment there is provided an antibody, or antigen binding fragment thereof, comprising a chimeric heavy chain constant region. In an embodiment, the antibody, or antigen binding fragment thereof, comprises an IgG1/IgG3 chimeric heavy chain constant region, such that the antibody, or antigen binding fragment thereof, has an enhanced effector function, for example enhanced ADCC or enhanced CDC, or enhanced ADCC and CDC functions. For example, a chimeric antibody, or antigen binding fragment thereof, of the invention may comprise at least one CH2 domain from IgG3. In one such embodiment, the antibody, or antigen binding fragment thereof, comprises one CH2 domain from IgG3 or both CH2 domains may be from IgG3. In a further embodiment, the chimeric antibody, or antigen binding fragment thereof, comprises an IgG1 CH1 domain, an IgG3 CH2 domain, and an IgG3 CH3 domain. In a further embodiment, the chimeric antibody, or antigen binding fragment thereof, comprises an IgG1 CH1 domain, an IgG3 CH2 domain, and an IgG3 CH3 domain except for position 435 that is histidine.

In a further embodiment, the chimeric antibody, or antigen binding fragment thereof, comprises an IgG1 CH1 domain and at least one CH2 domain from IgG3. In an embodiment, the chimeric antibody, or antigen binding fragment thereof, comprises an IgG1 CH1 domain and the following residues, which correspond to IgG3 residues, in a CH2 domain: 274Q, 276K, 296F, 300F and 339T. In an embodiment, the chimeric antibody, or antigen binding fragment thereof, also comprises 356E, which corresponds to an IgG3 residue, within a CH3 domain. In an embodiment, the antibody, or antigen binding fragment thereof, also comprises one or more of the following residues, which correspond to IgG3 residues within a CH3 domain: 358M, 384S, 392N, 397M, 4221, 435R, and 436F.

Also provided is a method of producing an antibody, or antigen binding fragment thereof, according to the invention comprising the steps of:
a) culturing a recombinant host cell comprising an expression vector comprising a nucleic acid sequence encoding a chimeric Fc region having both IgG1 and IgG3 Fc region amino acid residues (e.g. as described above); and
b) recovering the antibody, or antigen binding fragment thereof.

Such methods for the production of antibody, or antigen binding fragment thereof, with chimeric heavy chain constant regions can be performed, for example, using the COMPLEGENT technology system available from BioWa, Inc. (Princeton, NJ) and Kyowa Hakko Kirin Co., Ltd. The COMPLEGENT system comprises a recombinant host cell comprising an expression vector in which a nucleic acid sequence encoding a chimeric Fc region having both IgG1 and IgG3 Fc region amino acid residues is expressed to produce an antibody, or antigen binding fragment thereof, having enhanced CDC activity, i.e. CDC activity is increased relative to an otherwise identical antibody, or antigen binding fragment thereof, lacking such a chimeric Fc region, as described in WO 2007/011041 and US 2007/0148165, each of which are incorporated herein by reference. In an alternative embodiment, CDC activity may be increased by introducing sequence specific mutations into the Fc region of an IgG chain. Those of ordinary skill in the art will also recognize other appropriate systems.

The present invention also provides a method of producing an antibody, or antigen binding fragment thereof, according to the invention comprising the steps of:
a) culturing a recombinant host cell comprising an expression vector comprising a nucleic acid encoding the antibody, or antigen binding fragment thereof, optionally wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO 00/61739 and WO 02/31240, all of which are incorporated herein by reference. Those of ordinary skill in the art will also recognize other appropriate systems.

In one embodiment, the antibody, or antigen binding fragment thereof, is produced in a host cell in which the FUT8 gene has been inactivated. In a further embodiment, the antibody, or antigen binding fragment thereof, is produced in a −/− FUT8 host cell. In a further embodiment, the antibody, or antigen binding fragment thereof, is afucosylated at Asn297 (IgG1).

It will be apparent to those skilled in the art that such modifications may not only be used alone but may be used in combination with each other in order to further enhance effector function.

In one such embodiment, there is provided an antibody, or antigen binding fragment thereof, comprising a heavy chain constant region that comprises a both a mutated and chimeric heavy chain constant region, individually described above. For example, an antibody, or antigen binding fragment thereof, comprising at least one CH2 domain from IgG3 and one CH2 domain from IgG1, and wherein the IgG1 CH2 domain has one or more mutations at positions selected from 239, 332 and 330 (for example the mutations may be selected from S239D, 1332E and A330L), such that the antibody, or antigen binding fragment thereof, has enhanced effector function, e.g. enhanced ADCC or enhanced CDC, or enhanced ADCC and enhanced CDC in comparison to an equivalent antibody, or antigen binding fragment thereof, with an IgG1 heavy chain constant region lacking said mutations. In one embodiment, the IgG1 CH2 domain has the mutations S239D and 1332E. In another embodiment, the IgG1 CH2 domain has the mutations S239D, A330L, and 1332E.

In an alternative embodiment, there is provided an antibody, or antigen binding fragment thereof, comprising both a chimeric heavy chain constant region and an altered glycosylation profile, as individually described above. In an embodiment, the antibody, or antigen binding fragment thereof, comprises an altered glycosylation profile such that the ratio of fucose to mannose is 0.8:3 or less. In one such embodiment, the heavy chain constant region comprises at least one CH2 domain from IgG3 and one CH2 domain from IgG1 and has an altered glycosylation profile such that the ratio of fucose to mannose is 0.8:3 or less, for example wherein the antibody, or antigen binding fragment thereof, is defucosylated. Said antibody, or antigen binding fragment thereof, has an enhanced effector function, e.g. enhanced ADCC or enhanced CDC, or enhanced ADCC and enhanced CDC, in comparison to an equivalent antibody, or antigen binding fragment thereof, with an IgG1 heavy chain constant region lacking said glycosylation profile.

In an alternative embodiment, the antibody, or antigen binding fragment thereof, has at least one IgG3 heavy chain CH2 domain and at least one heavy chain constant domain from IgG1 wherein both IgG CH2 domains are mutated in accordance with the limitations described herein.

In one aspect, there is provided a method of producing an antibody, or antigen binding fragment thereof, according to the invention described herein comprising the steps of:
a) culturing a recombinant host cell containing an expression vector comprising a nucleic acid sequence encoding a chimeric Fc domain having both IgG1 and IgG3 Fc domain amino acid residues (e.g. as described above); and wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the ACCRETAMAB technology system available from BioWa, Inc. (Princeton, NJ) that combines the POTELLIGENT and COMPLEGENT technology systems to produce an antibody, or antigen binding fragment thereof, having both enhanced ADCC and CDC activity relative to an otherwise identical monoclonal antibody that lacks a chimeric Fc domain and that is fucosylated.

In another embodiment, there is provided an antibody, or antigen binding fragment thereof, comprising a mutated and chimeric heavy chain constant region wherein said antibody, or antigen binding fragment thereof, has an altered glycosylation profile such that the antibody, or antigen binding fragment thereof, has enhanced effector function, e.g. enhanced ADCC or enhanced CDC, or both enhanced ADCC and CDC. In one embodiment the mutations are selected from positions 239, 332 and 330, e.g. S239D, 1332E and A330L. In a further embodiment the heavy chain constant region comprises at least one CH2 domain from IgG3 and one CH1 domain from IgG1. In one embodiment the heavy chain constant region has an altered glycosylation profile such that the ratio of fucose to mannose is 0.8:3 or less, e.g. the antibody, or antigen binding fragment thereof, is defucosylated, such that said antibody, or antigen binding fragment thereof, has an enhanced effector function in comparison with an equivalent non-chimeric antibody, or antigen binding fragment thereof, lacking said mutations and lacking said altered glycosylation profile.

In a further embodiment, the anti-cotinine antibody, or antigen binding fragment thereof, comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In a further embodiment, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/1332E or S239D/1332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/1332E, wherein residue numbering is according to the EU Index.

In a further embodiment, the anti-cotinine antibody, or antigen binding fragment thereof, comprises a heavy chain variable region (VH) having SEQ ID NO: 7, a light chain variable region (VL) having SEQ ID NO: 8. In a further embodiment, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/1332E or S239D/1332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody has a heavy chain comprising SEQ ID NO: 9 and a light chain comprising SEQ ID NO: 10.

The present disclosure also provides a pharmaceutical composition comprising an anti-cotinine antibody, or antigen binding fragment thereof as disclosed herein, and a pharmaceutically acceptable excipient, carrier, or diluent.

The present disclosure also provides a combination comprising the compound of Formulae (I) or (II) as disclosed herein, preferably a compound of Formula (I) as disclosed herein, and an anti-cotinine antibody, or antigen-binding fragment thereof as disclosed herein. The compound of Formulae (I) or (II) and anti-cotinine antibody, or antigen binding fragment thereof can be present in the same composition or in separate compositions. In one embodiment, a combination comprises a pharmaceutical composition comprising the compound of Formulae (I) or (II) as disclosed herein, preferably a compound of Formula (I) as disclosed herein, and an anti-cotinine antibody, or antigen binding fragment thereof as disclosed herein, and a pharmaceutically acceptable carrier, diluent, or excipient. In another embodiment, a combination comprises a first pharmaceutical composition comprising a compound of Formulae (I) or (II) as disclosed herein, preferably a compound of Formula (I) as disclosed herein, and a pharmaceutically acceptable carrier, diluent, or excipient; and a second pharmaceutical composition comprising an anti-cotinine antibody or antigen binding fragment thereof as disclosed herein, and a pharmaceutically acceptable carrier, excipient, or diluent.

### Statement of Use

The compounds of Formula (I) and pharmaceutically acceptable salts thereof are capable of simultaneously binding a cell surface-expressed FAP and an anti-cotinine antibody, or antigen binding fragment thereof to form a ternary complex for the treatment and/or prevention of diseases or disorders associated with FAP-expressing cells.

In one embodiment, the present disclosure provides a method of treating and/or preventing a disease or disorder in a patient in need thereof comprising administering to the patient a therapeutically effective amount of the compound of Formula (I), or a pharmaceutically acceptable salt thereof, and an anti-cotinine antibody, or antigen-binding fragment thereof, wherein the disease or disorder is selected from a cancer.

In a further embodiment, the compound and the antibody, or antigen-binding fragment thereof, are administered simultaneously. In a further embodiment, the compound and the antibody, or antigen-binding fragment thereof, are administered simultaneously from a single composition, including as a fixed-dose composition or by pre-mixing the compound and the antibody, or antigen-binding fragment thereof, prior to administration. For example, the compound and the antibody, or antigen-binding fragment thereof, can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the antibody, or antigen-binding fragment thereof, are administered simultaneously from two separate compositions.

In a further embodiment, the compound and the antibody, or antigen-binding fragment thereof, are administered sequentially.

In certain embodiments, the compound and the antibody, or antigen-binding fragment thereof, whether administered simultaneously or sequentially, may be administered by the same route or may be administered by different routes. In one embodiment, the compound and the antibody, or antigen-binding fragment thereof, are both administered intraveneously or subcutaneously, in the same composition or in separate compositions. In another embodiment, the compound is administered orally and the antibody or antigen-binding fragment thereof is administered intravenously or subcutaneously.

In a further embodiment, the compound and the antibody, or antigen-binding fragment thereof, are administered in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are present as a combination in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are administered at a dosage of compound of 0.0001 mg/kg to 1 mg/kg and antibody of 0.01 mg/kg to 100 mg/kg. For example, in a further embodiment, the compound is administered at a dosage of about 0.0001 mg/kg to about 0.0002 mg/kg, about 0.0002 mg/kg to about 0.0003 mg/kg, about 0.0003 mg/kg to about 0.0004 mg/kg, about 0.0004 mg/kg to about 0.0005 mg/kg, about 0.0005 mg/kg to about 0.001 mg/kg, about 0.001 mg/kg to about 0.002 mg/kg, about 0.002 mg/kg to about 0.003 mg/kg, about 0.003 mg/kg to about 0.004 mg/kg, about 0.004 mg/kg to about 0.005 mg/kg, about 0.005 mg/kg to about 0.01 mg/kg, about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, and/or about 0.5 mg/kg to about 1 mg/kg, and the antibody, or antigen-binding fragment thereof, is administered at a dosage of about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, and/or about 90 mg/kg to about 100 mg/kg.

In a further embodiment, the compound and the antibody, or antigen-binding fragment thereof, are administered at a dosage of compound of 0.007 mg to 70 mg and antibody of 0.7 mg to 7000 mg. For example, in a further embodiment, the compound is administered at a dosage of about 0.007 mg to about 0.01 mg, about 0.01 mg to about 0.02 mg, about 0.02 mg to about 0.03 mg, about 0.03 mg to about 0.04 mg, about 0.04 mg to about 0.05 mg, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, and/or about 60 mg to about 70 mg, and the antibody, or antigen-binding fragment thereof, is administered at a dosage of about 0.7 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 100 mg, about 100 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, and/or about 6500 mg to about 7000 mg.

In a further embodiment, the compound and the antibody, or antigen-binding fragment thereof, are administered in a molar ratio and/or dosage as described herein once every week, once every two weeks, once every three weeks, once every four weeks, once every five weeks, or once every six weeks for a period of one week to one year, such as a period of one week, one month, two months, three months, four months, five months, six months, seven months, eight months, nine months, ten months, eleven months, or twelve months.

In a further embodiment, the present disclosure provides a therapeutically effective amount of the compound of Formula (I), or a pharmaceutically acceptable salt thereof, and an anti-cotinine antibody, or antigen-binding fragment thereof for use in therapy. The compound of Formula (I), or a pharmaceutically acceptable salt thereof, and anti-cotinine antibody, or antigen-binding fragment thereof can be used in treating or preventing a disease or disorder selected from a cancer.

In a further embodiment, the present disclosure provides a therapeutically effective amount of the compound of Formula (I), or a pharmaceutically acceptable salt thereof, and an anti-cotinine antibody, or antigen-binding fragment thereof for the manufacture of a medicament. The medicament can be used in treating or preventing a disease or disorder selected from a cancer.

In a further embodiment, the disease or disorder is mediated by FAP and/or is associated with FAP-positive pathogenic cells. In a further embodiment, FAP-positive cell types are identified by testing for expression of FAP by immunohistochemistry or flow cytometry.

In a further embodiment, the disease or disorder is a cancer selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, bladder cancer, bone cancer, esophogeal cancer, gastric cancer, renal cancer, melanoma cancer, thyroid cancer, or breast cancer, preferably pancreatic cancer or CRC.

In a further embodiment, the disease or disorder is a solid tumor. In a further embodiment, the disease or disorder is a solid tumor selected from lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, bladder cancer, bone cancer, esophogeal cancer, gastric cancer, renal cancer, melanoma cancer, thyroid cancer, or breast cancer, preferably a solid tumor selected from pancreatic cancer or CRC.

In a further embodiment, the disease or disorder is a PD-1 relapsed or refractory cancer, such as a PD-1 relapsed or refractory lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, bladder cancer, bone cancer, esophogeal cancer, gastric cancer, renal cancer, melanoma cancer, thyroid cancer, or breast cancer, preferably a PD-1 relapsed or refractory pancreatic cancer or CRC.

In a further embodiment, the disease or disorder is a non-solid cancer. In a further embodiment, the disease or disorder is a leukemia, a lymphoma, or a myeloma.

In one embodiment, the present disclosure provides a method of increasing antibody-dependent cell cytotoxicity (ADCC) of FAP-expressing cells comprising contacting the cells with an effective amount of the compound of Formula (I), or pharmaceutically acceptable salt thereof, and an anti-cotinine antibody, or antigen-binding fragment thereof, wherein the FAP-binding moiety of the compound binds the FAP expressed on the cells.

In one embodiment, the present disclosure provides a method of increasing antibody dependent cellular phagocytosis (ADCP) of FAP-expressing cells comprising contacting the cells with an effective amount of the compound of Formula (I), or pharmaceutically acceptable salt thereof, and an anti-cotinine antibody, or antigen-binding fragment thereof, wherein the FAP-binding moiety of the compound binds the FAP expressed on the cells.

In one embodiment, the present disclosure provides a method of increasing complement dependant cytotoxicity (CDC) of FAP-expressing cells comprising contacting the cells with an effective amount of the compound of Formula (I), or pharmaceutically acceptable salt thereof, and an anti-cotinine antibody, or antigen-binding fragment thereof, wherein the FAP-binding moiety of the compound binds the FAP expressed on the cells.

In one embodiment, the present disclosure provides a method of conditioning a patient for therapy with a chimeric antigen receptor (CAR) T cell therapy, comprising administering to a patient an effective amount of the compound of Formula (I), or pharmaceutically acceptable salt thereof, and an anti-cotinine antibody, or antigen-binding fragment thereof. In some embodiments, the compound of Formula (I), or pharmaceutically acceptable salt thereof, and an anti-cotinine antibody, or antigen-binding fragment thereof are administered in combination with the CAR-T cell therapy. A compound of Formula (I), or pharmaceutically acceptable salt thereof, and an anti-cotinine antibody, or antigen-binding fragment thereof may be administered as a conditioning therapy or combination therapy to improve efficacy in treatment of solid tumor cancers. In other embodiments, a compound of Formula (I), or pharmaceutically acceptable salt thereof, and an anti-cotinine antibody, or antigen-binding fragment thereof may be administered as a neoadjuvant treatment for other therapies, including but not limited to immunotherapy, surgical resection, radiation, and/or chemotherapy.

In one embodiment, the present disclosure provides a method of depleting FAP-expressing cells comprising contacting the cells with the compound of Formula (I), or pharmaceutically acceptable salt thereof, and an anti-cotinine antibody, or antigen-binding fragment thereof, wherein the FAP-binding moiety of the compound binds the FAP expressed on the cells.

In a further embodiment, the FAP-expressing cells are pathogenic cells.

In a further embodiment, the pathogenic cell is a tumor cell or cancer cell, an immune cell, or a stromal cell.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, bladder cancer cells, bone cancer cells, esophogeal cancer cells, gastric cancer cells, renal cancer cells, melanoma cancer cells, thyroid cancer cells, or breast cancer cells, preferably pancreatic cancer cells or CRC cells.

In a further embodiment, the immune cells are macrophages.

In a further embodiment, the stromal cells are cancer associated fibroblasts (CAFs).

### Combination Therapies

The compounds of the invention may be employed alone or in combination with other therapeutic agents. Combination therapies according to the present invention thus comprise the administration of at least one compound of Formula (I) or a pharmaceutically acceptable salt thereof, and the use of at least one other pharmaceutically active agent. The compounds of the invention and the other pharmaceutically active agents may be administered together in a single pharmaceutical composition or separately and, when administered separately this may occur simultaneously or sequentially in any order. The amounts of the compounds of the invention and the other pharmaceutically active agents and the relative timings of administration will be selected in order to achieve the desired combined therapeutic effect.

It will be appreciated that when the compound of the present invention is administered in combination with one or more other therapeutically active agents normally administered by the inhaled, intravenous, oral, intranasal, ocular topical or other route, that the resultant pharmaceutical composition may be administered by the same route. Alternatively, the individual components of the composition may be administered by different routes.

In one embodiment, the compounds and pharmaceutical composition disclosed herein are used in combination with, or include, one or more additional therapeutic agents. In a further embodiment, the additional therapeutic agent is a checkpoint inhibitor or an immune modulator.

In a further embodiment, the checkpoint inhibitor is selected from a PD-1 inhibitor (e.g., an anti-PD-1 antibody including, but not limited to, pembrolizumab, nivolumab, cemiplimab, or dostarlimab), a PD-L1 inhibitor (e.g., an anti-PD-L1 antibody including, but not limited to, atezolizumab, avelumab, or durvalumab), or a CTLA-4 inhibitor (e.g., an anti-CTLA-4 antibody including, but not limited to, ipilimumab or tremilumumab).

In a further embodiment, the checkpoint inhibitor is selected from a CD226 axis inhibitor, including but not limited to a TIGIT inhibitor (e.g., an anti-TIGIT antibody), a CD96 inhibitor (e.g., an anti-CD96 antibody), and/or a PVRIG inhibitor (e.g., an anti-PVRIG antibody).

In a further embodiment, the immune modulator is an ICOS agonist (e.g., an anti-ICOS antibody including, but not limited to feladilimab), a PARP inhibitor (e.g., niraparib, olaparib), or a STING agonist.

### Pharmaceutical Compositions, Dosages, and Dosage Forms

For the purposes of administration, in certain embodiments, the ARMs described herein are administered as a raw chemical or are formulated as pharmaceutical compositions. Pharmaceutical compositions disclosed herein include an ARM and one or more of: a pharmaceutically acceptable carrier, diluent or excipient. An ARM is present in the composition in an amount which is effective to treat a particular disease, disorder or condition of interest. The activity of the ARM can be determined by one skilled in the art, for example, as described in the biological assays described below. Appropriate concentrations and dosages can be readily determined by one skilled in the art. In certain embodiments, the ARM is present in the pharmaceutical composition in an amount from about 25 mg to about 500 mg. In certain embodiments, the ARM is present in the pharmaceutical composition in an amount of about 0.01 mg to about 300 mg. In certain embodiments, ARM is present in the pharmaceutical composition in an amount of about 0.01 mg, 0.1 mg, 1 mg, 5 mg, 10 mg, 25 mg, 50 mg, 100 mg, 200 mg, 300 mg, 400 mg or about 500 mg.

Administration of the compounds of the invention, or their pharmaceutically acceptable salts, in pure form or in an appropriate pharmaceutical composition, is carried out via any of the accepted modes of administration of agents for serving similar utilities. The pharmaceutical compositions of the invention are prepared by combining a compound of the invention with an appropriate pharmaceutically acceptable carrier, diluent or excipient, and in specific embodiments are formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral (e.g., intramuscular, subcutaneous, intravenous, or intradermal), sublingual, buccal, rectal, vaginal, and intranasal. Pharmaceutical compositions of the invention are formulated so as to allow the active ingredients contained therein to be bioavailable upon administration of the composition to a patient.

Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia. College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings described herein.

The pharmaceutical compositions disclosed herein are prepared by methodologies well known in the pharmaceutical art. For example, in certain embodiments, a pharmaceutical composition intended to be administered by injection is prepared by combining a compound of the invention with sterile, distilled water so as to form a solution. In some embodiments, a surfactant is added to facilitate the formation of a homogeneous solution or suspension. Surfactants are compounds that non-covalently interact with the compound of the invention so as to facilitate dissolution or homogeneous suspension of the compound in the aqueous delivery system.

Traditional antibody therapeutics have several disadvantages that are addressed by the ARMs approach described herein including difficulties in managing adverse events via adjusting dose and dose frequency of administration, challenges in generating antibodies to certain classes of drug targets (e.g., GPCRs, ion channels, and enzymes), and a new cell line for development is required for each new antibody which can be slow and costly. Moreover, different formats of biologics (e.g., bispecifics) can be challenging to manufacture. In contrast, the ARMs approach provides the following advantages: uniting the pharmacology of antibodies with the dose-control of small molecules, dose controlled PK/PD allowing temporal cell depletion, simpler multimerization, and rapid reversal of cell depletion through dosing of the antibody-binding component (e.g., cotinine hapten) which can uncouple therapeutic effects from potential adverse events.

### EXAMPLES

The following examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made. References to preparations carried out in a similar manner to, or by the general method of, other preparations, may encompass variations in routine parameters such as time, temperature, workup conditions, minor changes in reagent amounts etc. Chemical names for all title compounds were generated using ChemDraw Plug-in version 16.0.1.13c (90) or ChemDraw desktop version 16.0.1.13 (90). A person of ordinary skill in the art will recognize that compounds of the invention may have alternative names when different naming software is used.

### Compound Synthesis

The compounds according to Formulae (I) and (II) are prepared using conventional organic synthetic methods. A suitable synthetic route is depicted below in the following general reaction schemes. All the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

### Scheme 1

Intermediate 1: (2S,3S)-1-Methyl-5-oxo-2-(pyridin-3-yl)pyrrolidine-3-carboxylic acid

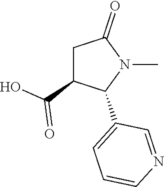 [SMILES: CN1C(=O)C[C@H](C(=O)O)[C@H]1c1cccnc1]

Commercially available, racemic trans-4-cotininecarboxylic acid (304 g, 1.38 mol) was purified by chiral prep HPLC (61 injections) on Chiralpak 1A 20u 101×210 mm at 500 mL/min eluting with 50% acetonitrile in methanol containing 0.1% formic acid. The desired fractions were collected and were concentrated at 45° C. The solid residue was stirred in acetonitrile, was filtered, and was dried under reduced pressure for 18 h to provide the title compound as a white solid (143.6 g, 652 mmol, 94.5% yield). Analytical chiral HPLC: 95% ee at ret. time 2.5 min, Chiralpak AD-H 5u 4.6×150 mm, methanol with 0.1% formic acid at 1.0 mL/min; Alpha D=+58 deg (c=0.3, CH₃OH); VCD analysis was used to assign absolute stereochemistry. LC-MS m/z 221.1 (M+H)+. ¹H NMR (400 MHz, DMSO-d₆) δ ppm 2.48-2.49 (m, 2H) 2.53-2.61 (m, 1H) 2.71-2.80 (m, 1H) 3.06-3.15 (m, 1H) 3.34 (br s, 1H) 4.79 (d, J=6.3 Hz, 1H) 7.35-7.57 (m, 1H) 7.74 (dt, J=7.9, 2.0 Hz, 1H) 8.54 (d, J=1.8 Hz, 1H) 8.57 (dd, J=4.7, 1.7 Hz, 1H) 12.78 (br s, 1H).

### Intermediate 2: (1R,4r)-4-(4-(((1S,4R)-4-(2-((2S,3S)-1-Methyl-5-oxo-2-(pyridin-3-yl) pyrrolidine-3-carboxamido)ethoxy)cyclohexyl)oxy)butanamido)cyclohexane-1-carboxylic acid, Hydrochloride salt

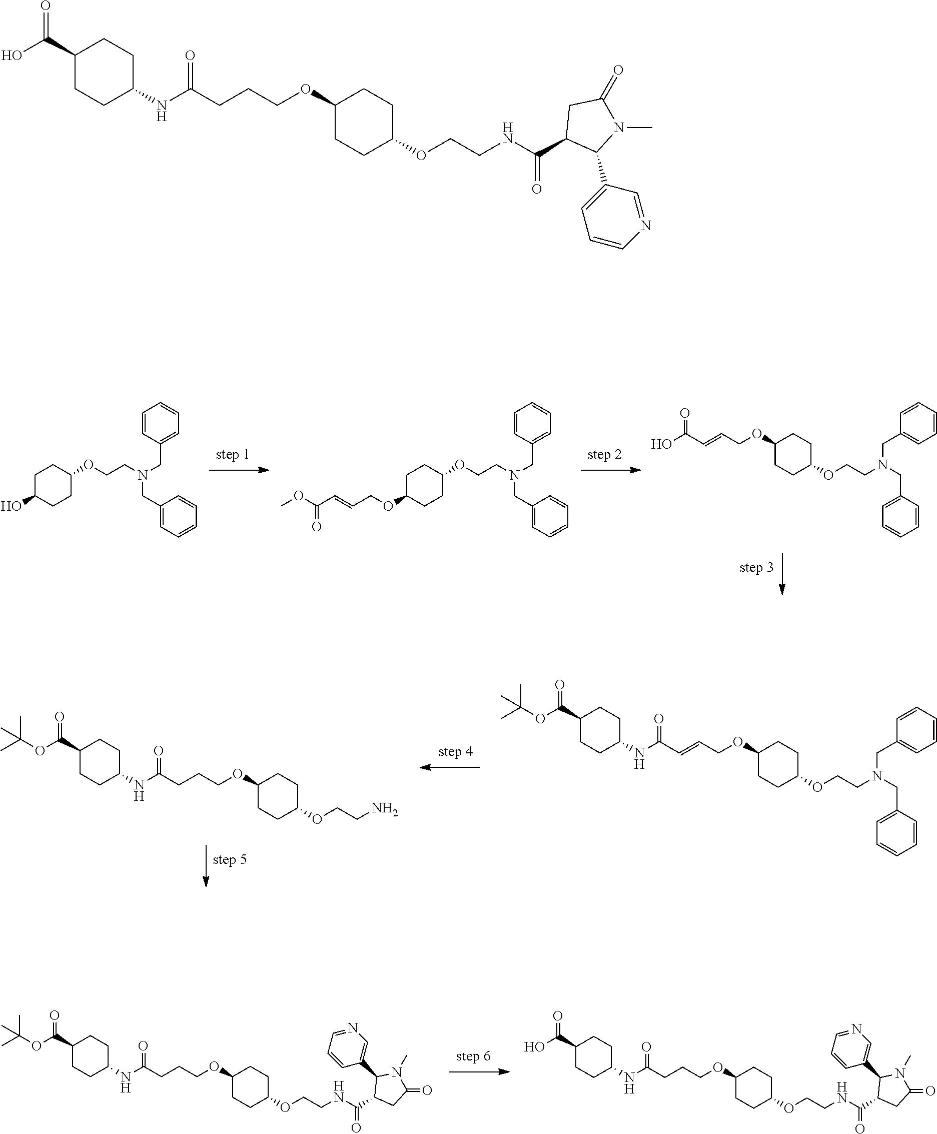

### Step 1: methyl (E)-4-(((1r,4r)-4-(2-(Dibenzylamino)ethoxy)cyclohexyl)oxy)but-2-enoate

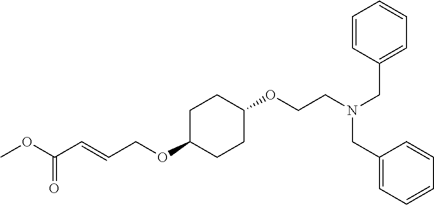 [SMILES: COC(=O)/C=C/CO[C@H]1CC[C@H](OCCN(Cc2ccccc2)Cc2ccccc2)CC1]

To a stirred solution of (1r,4r)-4-(2-(dibenzylamino)ethoxy)cyclohexan-1-ol (250 g, 736 mmol) in toluene (2500 mL) were added methyl but-2-ynoate (140 g, 1423 mmol), triphenylphosphine (19.32 g, 73.6 mmol), and acetic acid (16.86 mL, 295 mmol) at RT and the resulting solution was stirred at 115° C. for 16 h. The reaction mixture was cooled to room temperature and concentrated under reduced pressure to the crude compound. The crude compound was adsorbed on silicagel (500 g, 60-120 mesh), and purified by manual column chromatography(1.5 kg, 100-200 mesh) eluted with 15% EtOAc in pet-ether to afford methyl (E&Z)-4-(((1r,4r)-4-(2-(dibenzylamino)ethoxy)cyclohexyl)oxy)but-2-enoate (350 g) as a mixture of E/Z isomers (52.48% and 21.15%). To separate both isomers, the compound was adsorbed on silica gel (500 g, 100-200 mesh), and purified by manual column chromatography(1.5 kg, 100-200 mesh) eluted with 15% EtOAc in pet-ether to afford the title compound (240 g, 463 mmol, 62.9% yield, 84.45% purity) as a pale yellow liquid. LC-MS m/z 438.3 (M+H)+.

Step 2: (E)-4-(((1,4-trans)-4-(2-(Dibenzylamino)ethoxy)cyclohexyl)oxy)but-2-enoic acid

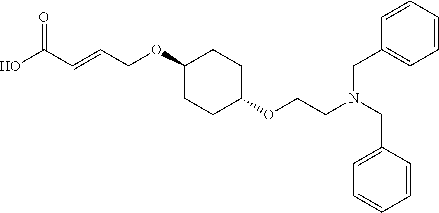 [SMILES: O=C(O)/C=C/CO[C@H]1CC[C@H](OCCN(Cc2ccccc2)Cc2ccccc2)CC1]

Methyl (E)-4-(((1,4-trans)-4-(2-(dibenzylamino)ethoxy)cyclohexyl)oxy)but-2-enoate (9.03 g, 20.64 mmol) was dissolved in tetrahydrofuran (THF) (25 mL) and aqueous 5.089 Molar sodium hydroxide (4.87 mL, 24.76 mmol) was added. The homogenous pale-yellow reaction was heated at reflux for 1 hour. Additional 5.089 M sodium hydroxide (1.217 mL, 6.19 mmol) was added and the reaction was refluxed for 50 minutes. The reaction was cooled over 60 minutes and concentrated in vacuo. The residue was azeotroped twice with toluene in order to aid in removal of water. The residue was pumped under high vacuum to afford the title compound (9.9 g, 22.17 mmol, 107% yield, 82% purity, E/Z mixture) as a yellow solid. LC-MS m/z 424.4 (M+H)+.

### Step 3: tert-butyl (1R,4r)-4-((E)-4-(((1r,4R)-4-(2-(Dibenzylamino)ethoxy)cyclohexyl)oxy) but-2-enamido)cyclohexane-1-carboxylate

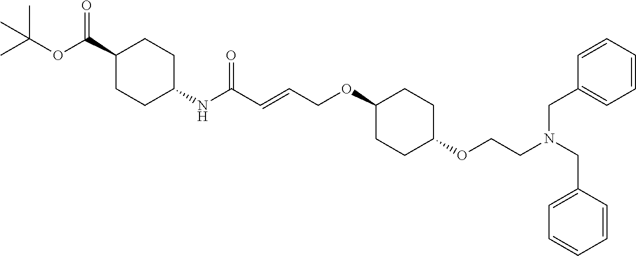 [SMILES: CC(C)(C)OC(=O)[C@H]1CC[C@H](NC(=O)/C=C/CO[C@H]2CC[C@H](OCCN(Cc3ccccc3)Cc3ccccc3)CC2)CC1]

(E)-4-(((1,4-trans)-4-(2-(dibenzylamino)ethoxy)cyclohexyl)oxy)but-2-enoic acid, sodium salt (9.2 g, 20.60 mmol) was suspended in dry DMF (40 ml) with stirring. HATU (8.62 g, 22.66 mmol) was added as a solid and a partially dissolved mixture was observed. The mixture was stirred for 30 minutes to give a partially dissolved greenish solution. tert-butyl (1,4-trans)-4-aminocyclohexane-1-carboxylate (4.11 g, 20.60 mmol) was added as a solution in DMF (10 ml) followed by addition of a solution of DIEA (10.80 mL, 61.8 mmol) in DMF (10 ml). An additional 10 ml of DMF was added and the heterogeneous mixture was stirred for 15 hours at room temperature. Additional HATU (1.724 g, 4.53 mmol) was added and the almost homogeneous reaction was stirred for 60 minutes. The cloudy reaction was stirred for an additional 60 minutes. The reaction was diluted with 200 ml of EtOAc and 200 ml of water and stirred for 10 minutes. The resulting homogeneous biphasic mixture was transferred to a separatory funnel and the layers were separated. The aqueous layer was extracted twice more with 150 ml EtOAc and the combined EtOAc layers were washed 4× with water and 2× with saturated NaCl in order to remove DMF. The EtOAc extracts were dried over sodium sulfate, filtered, and concentrated in vacuo, and pumped under high vacuum to give an orange syrup which was purified via silica-gel chromatography (Isco Combiflash, 330 gram gold column, 0-80% EtOAc:heptane over 45 minutes, 150 ml/min flow rate, loaded as a solution in DCM) to give the title compound (4.55 g, 7.52 mmol, 36.5% yield) as a white foamy solid. LC-MS m/z 605.5 (M+H)+.

Step 4: tert-butyl (1R,4r)-4-(4-(((1r,4R)-4-(2-Aminoethoxy)cyclohexyl)oxy)butanamido) cyclohexane-1-carboxylate

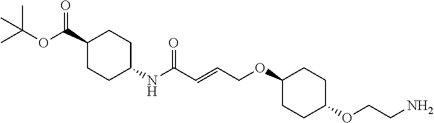 [SMILES: CC(C)(C)OC(=O)[C@H]1CC[C@H](NC(=O)/C=C/CO[C@H]2CC[C@H](OCCN)CC2)CC1]

A 500 ml 3-necked RB flask was charged with tert-butyl (1R,4r)-4-((E)-4-(((1r,4R)-4-(2-(dibenzylamino)ethoxy)cyclohexyl)oxy)but-2-enamido)cyclohexane-1-carboxylate (6.40 g, 10.58 mmol) and isopropanol (120 mL) and the suspension was stirred until a homogeneous solution was obtained. 10% wet Pd—C(0.640 g, 6.01 mmol) was added and the flask was evacuated and placed under 2 balloons of hydrogen attached to the end necks of the flask. The middle neck was covered with a rubber septum. The reaction was stirred at room temperature for 16 hours. The reaction was degassed and filtered 2× through celite. The filtrate was concentrated in vacuo and pumped under high vacuum to give a waxy grey solid with a slight odor of isopropanol. The waxy solid was dissolved in DCM and concentrated in vacuo at 54 degrees C. for 20 minutes in order to aid in removal of isopropanol. The residue was pumped under high vacuum to afford the title compound (4.44 g, 10.41 mmol, 98% yield) as a waxy grey solid. LC-MS m/z 427.4 (M+H)+.

Step 5: tert-butyl (1R,4r)-4-(4-(((1S,4R)-4-(2-((2S,3S)-1-Methyl-5-oxo-2-(pyridin-3-yl)pyrrolidine-3-carboxamido)ethoxy)cyclohexyl)oxy)butanamido)cyclohexane-1-carboxylate.

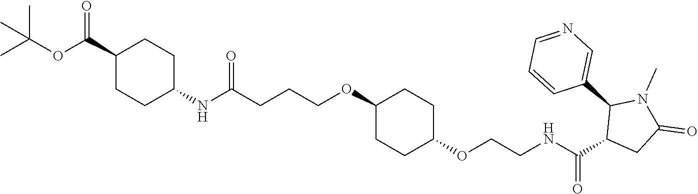 [SMILES: CN1C(=O)C[C@H](C(=O)NCCO[C@H]2CC[C@H](OCCCC(=O)N[C@H]3CC[C@H](C(=O)OC(C)(C)C)CC3)CC2)[C@H]1c1cccnc1]

(2S,3S)-1-Methyl-5-oxo-2-(pyridin-3-yl)pyrrolidine-3-carboxylic acid (Intermediate 1) (2.287 g, 10.38 mmol) was suspended in 30 ml DCM with stirring in a 500 ml RB flask at room temperature. HATU (4.34 g, 11.42 mmol) was added and the suspension was stirred for 15 minutes. A solution of tert-butyl (1R,4r)-4-(4-(((1r,4R)-4-(2-aminoethoxy)cyclohexyl)oxy) butanamido)cyclohexane-1-carboxylate (4.43 g, 10.38 mmol) in DCM (20 ml) was added dropwise via pipet over 15 minutes. After addition was complete, a solution of DIEA (5.44 mL, 31.2 mmol) in DCM (10 ml) was added dropwise over 10 minutes and the resulting homogeneous dark solution was stirred at room temperature for 16 hours. The reaction was concentrated in vacuo in order to remove DCM and DIEA. The residue was dissolved in 100 ml DCM and transferred to a separatory funnel. 20 ml of saturated sodium bicarbonate was added The layers were separated and the DCM layer was washed with saturated NaCl, dried over sodium sulfate, filtered, concentrated in vacuo, and pumped under high vacuum to give an orange syrup which was purified via silica gel chromatography (Isco Combiflash, 0-10% MeOH:DCM over 60 minutes, 330 gram gold column, 150 ml/min flow rate, loaded as a solution in 30 ml DCM) to afford the title compound (4.13 g, 6.57 mmol, 63.2% yield) as a white solid. LC-MS m/z 629.3 (M+H)+.

Step 6: (1R,4r)-4-(4-(((1S,4R)-4-(2-((2S,3S)-1-Methyl-5-oxo-2-(pyridin-3-yl)pyrrolidine-3-carboxamido)ethoxy)cyclohexyl)oxy)butanamido)cyclohexane-1-carboxylic acid, Hydrochloride salt.

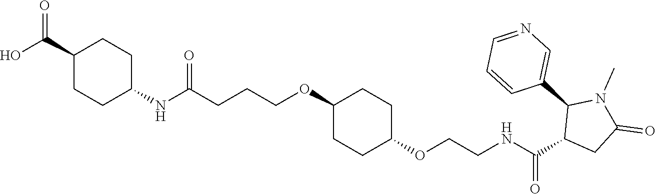 [SMILES: CN1C(=O)C[C@H](C(=O)NCCO[C@H]2CC[C@H](OCCCC(=O)N[C@H]3CC[C@H](C(=O)O)CC3)CC2)[C@H]1c1cccnc1]

tert-Butyl (1R,4r)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylate (4.13 g, 6.57 mmol) was dissolved in dry 1,4-dioxane (13 ml) with stirring in a 250 ml RB flask. 4 M anhydrous HCl (39 mL, 156 mmol) in 1,4-dioxane was added and the mixture was stirred at room temperature. Upon HCl addition, an insoluble oil was observed. The mixture was stirred for 90 minutes at room temperature. The reaction was concentrated in vacuo (at 60 degrees bath temperature) and pumped under high vacuum for 15 hours to afford the title compound (4.187 g, 6.87 mmol, 105% yield) as a white solid. LC-MS m/z 573.4 (M+H)+.

Intermediate 3: 4-(((1R,4r)-4-(2-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexane-1-carboxamido)ethoxy)cyclohexyl)oxy)butanoic acid

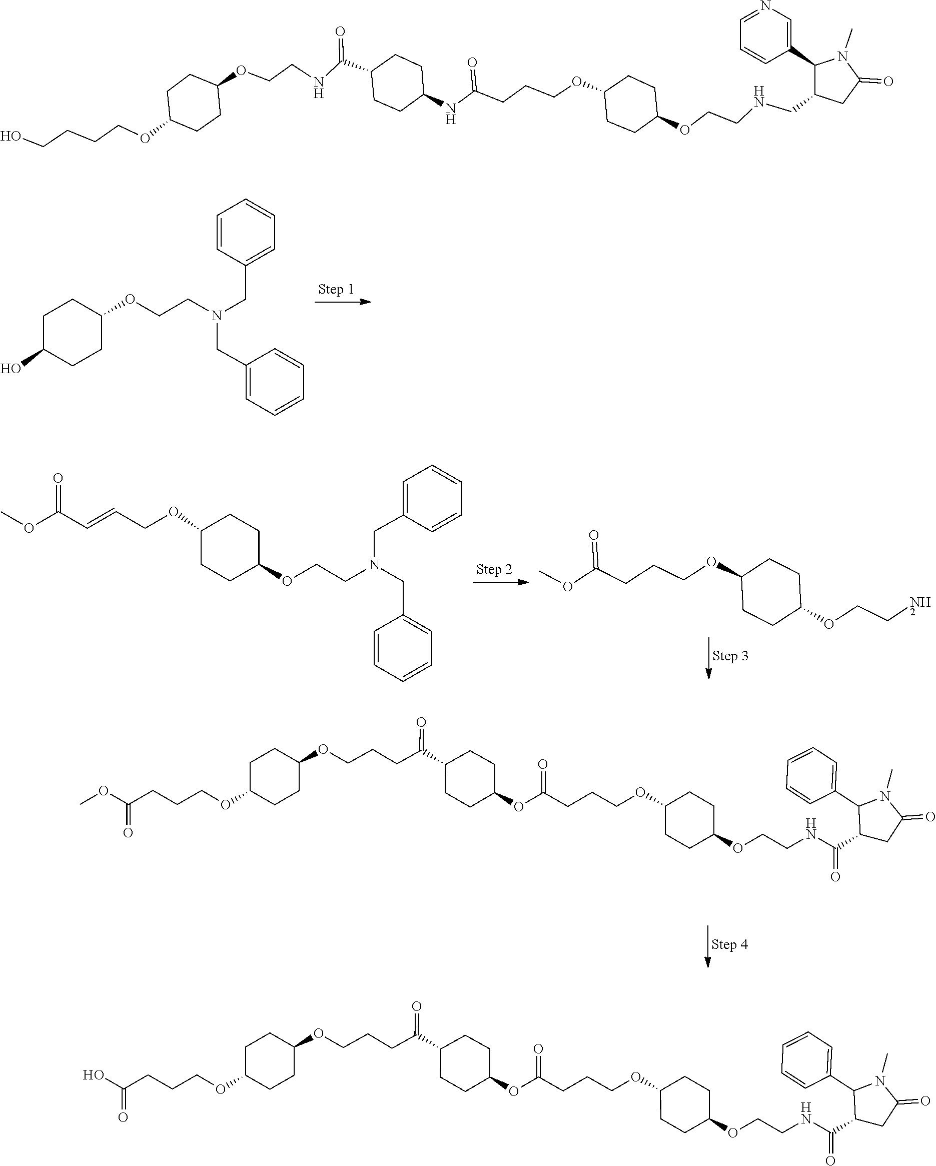 [SMILES: CN1C(=O)C[C@H](CNCCO[C@H]2CC[C@H](OCCCC(=O)N[C@H]3CC[C@H](C(=O)NCCO[C@H]4CC[C@H](OCCCCO)CC4)CC3)CC2)[C@H]1c1cccnc1 ; O[C@H]1CC[C@H](OCCN(Cc2ccccc2)Cc2ccccc2)CC1 ; COC(=O)/C=C/CO[C@H]1CC[C@H](OCCN(Cc2ccccc2)Cc2ccccc2)CC1 ; COC(=O)CCCO[C@H]1CC[C@H](OCCN)CC1 ; COC(=O)CCCO[C@H]1CC[C@H](OCCCC(=O)[C@H]2CC[C@H](OC(=O)CCCO[C@H]3CC[C@H](OCCNC(=O)[C@H]4CC(=O)N(C)C4c4ccccc4)CC3)CC2)CC1 ; CN1C(=O)C[C@H](C(=O)NCCO[C@H]2CC[C@H](OCCCC(=O)O[C@H]3CC[C@H](C(=O)CCCO[C@H]4CC[C@H](OCCCC(=O)O)CC4)CC3)CC2)C1c1ccccc1]

Step 1: methyl (E)-4-(((1r,4r)-4-(2-(Dibenzylamino)ethoxy)cyclohexyl)oxy)but-2-enoate.

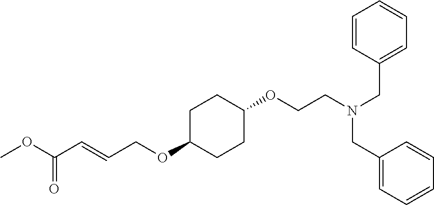 [SMILES: COC(=O)/C=C/CO[C@H]1CC[C@H](OCCN(Cc2ccccc2)Cc2ccccc2)CC1]

To a stirred solution of (1r,4r)-4-(2-(dibenzylamino)ethoxy)cyclohexan-1-ol (250 g, 736 mmol) in toluene (2500 mL) were added methyl but-2-ynoate (140 g, 1423 mmol), triphenylphosphine (19.32 g, 73.6 mmol), and acetic acid (16.86 mL, 295 mmol) at RT and the resulting solution was stirred at 115° C. for 16 h. The reaction mixture was cooled to room temperature and concentrated under reduced pressure to the crude compound. The crude compound was adsorbed on silicagel (500 g, 60-120 mesh), and purified by manual column chromatography(1.5 kg, 100-200 mesh) eluted with 15% EtOAc in pet-ether to afford methyl (E&Z)-4-(((1r,4r)-4-(2-(dibenzylamino)ethoxy)cyclohexyl)oxy)but-2-enoate (350 g) as a mixture of E/Z isomers (52.48% and 21.15%). To separate both isomers, the compound was adsorbed on silica gel (500 g, 100-200 mesh), and purified by manual column chromatography(1.5 kg, 100-200 mesh) eluted with 15% EtOAc in pet-ether to afford the title compound (240 g, 463 mmol, 62.9% yield, 84.45% purity) as a pale yellow liquid. LC-MS m/z 438.3 (M+H)+.

Step 2: Methyl 4-(((1R,4R)-4-(2-aminoethoxy)cyclohexyl)oxy)butanoate

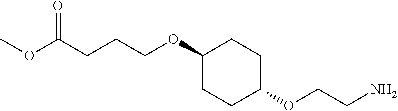 [SMILES: COC(=O)CCCO[C@H]1CC[C@H](OCCN)CC1]

A RB flask was charged with methyl (E)-4-(((1R,4R)-4-(2-(dibenzylamino)ethoxy)cyclohexyl)oxy)but-2-enoate (1.00 g, 2.28 mmol) and Ethanol (22.8 mL), and the suspension was stirred until a homogeneous light yellow solution was obtained. The reaction was evacuated and back-filled with nitrogen gas. 10% wet palladium (0.243 g, 2.28 mmol) was added. The reaction was evacuated and back-filled with a hydrogen gas balloon and stirred at RT overnight. The suspension was filtered through a celite plug, washing with additional EtOH (20 mL), concentrated in vacuo, and dried under vacuum overnight to provide the title compound as an off-white semi-solid (557 mg, 2.15 mmol, 93.9% yield). LC-MS m/z 260.2 (M+H)+.

Step 3: Methyl 4-(((1R,4r)-4-(2-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexane-1-carboxamido)ethoxy)cyclohexyl)oxy)butanoate

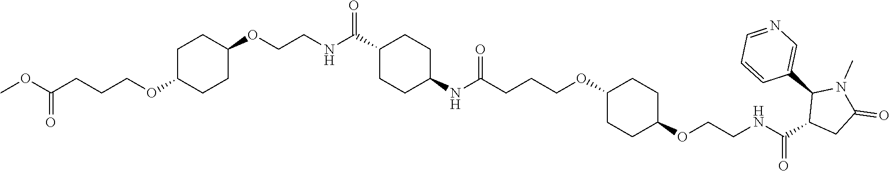 [SMILES: COC(=O)CCCO[C@H]1CC[C@H](OCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)CC1]

To a solution of methyl 4-(((1 r,4r)-4-(2-aminoethoxy)cyclohexyl)oxy)butanoate (110 mg, 0.424 mmol) and (1R,4r)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylic acid (243 mg, 0.424 mmol) in N,N-Dimethylformamide (DMF) (2121 μl) was added HATU (210 mg, 0.551 mmol), and DIPEA (222 μl, 1.272 mmol). The mixture was stirred at RT overnight. Diluted with 50 mL EtOAc and washed with sat NaHCO₃ (3×50 mL) and brine (50 mL). Dried over sodium sulfate, filtered, and concentrated. The residue was purified on a 12 g ISCO gold silica gel column eluting with 0-15% MeOH/DCM, staining with ninhydrin. Desired fractions were concentrated resulting in the title compound (81.4 mg, 0.036 mmol, 8.5% yield) as a tan oil of low purity. LC-MS m/z 814.6 (M+H)⁺.

### Step 4: 4-(((1R,4r)-4-(2-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxamido)ethoxy)cyclohexyl)oxy)butanoic acid

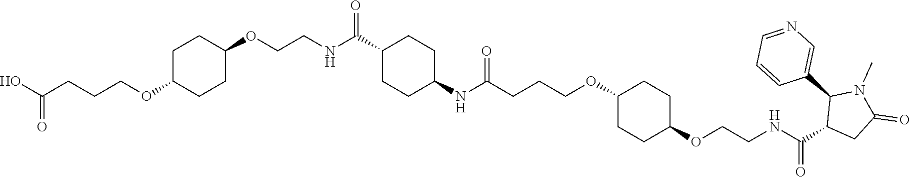 [SMILES: CN1C(=O)C[C@H](C(=O)NCCO[C@H]2CC[C@H](OCCCC(=O)N[C@H]3CC[C@H](C(=O)NCCO[C@H]4CC[C@H](OCCCC(=O)O)CC4)CC3)CC2)[C@H]1c1cccnc1]

To a round bottom flask containing methyl 4-(((1R,4r)-4-(2-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy) butanamido)cyclohexane-1-carboxamido)ethoxy)cyclohexyl)oxy)butanoate (105 mg, 0.129 mmol) was added 2M aqueous HCl (645 μl, 1.290 mmol). The mixture was heated to 50° C. for 4 hours.

Then concentrated, dried on high vac overnight resulting in the title compound (215 mg, 100% yield) (assumed theoretical yield as used crude without purification). LC-MS m/z 800.6 (M+H)⁺.

### Intermediate 4: 1-((2S,3S)-1-Methyl-5-oxo-2-(pyridin-3-yl)pyrrolidin-3-yl)-1-oxo-5,8,11,14,17,20,23,26,29,32,35,38-dodecaoxa-2-azahentetracontan-41-oic acid, Ammonia salt

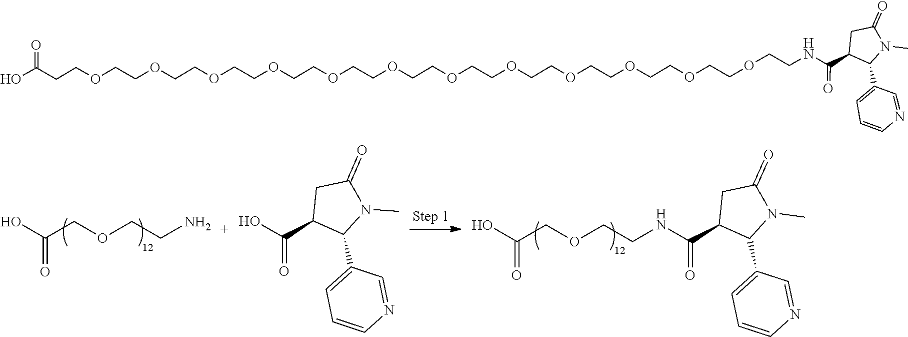

Step 1: 1-((2S,3S)-1-Methyl-5-oxo-2-(pyridin-3-yl)pyrrolidin-3-yl)-1-oxo-5,8,11,14,17,20,23,26,29,32,35,38-dodecaoxa-2-azahentetracontan-41-oic acid, Ammonia salt

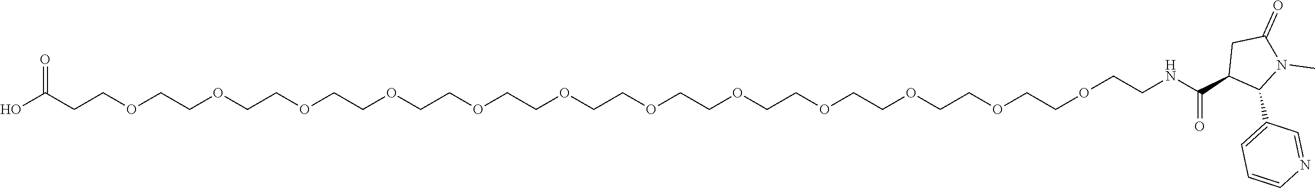 [SMILES: CN1C(=O)C[C@H](C(=O)NCCOCCOCCOCCOCCOCCOCCOCCOCCOCCOCCOCCOCCC(=O)O)[C@H]1c1cccnc1]

In a round bottom flask, (2S,3S)-1-methyl-5-oxo-2-(pyridin-3-yl)pyrrolidine-3-carboxylic acid (Intermediate 1) (0.357 g, 1.619 mmol) was dissolved in acetonitrile (10 ml). TSTU (0.487 g, 1.619 mmol) and DIPEA (1.131 ml, 6.48 mmol) were then added and the reaction was stirred at RT. Upon activation of the acid, 1-amino-3,6,9,12,15,18,21,24,27,30,33,36-dodecaoxanonatriacontan-39-oic acid (1 g, 1.619 mmol) was added as a solid and N,N-dimethylformamide (DMF) (3 ml) was added to aid in the solubility of the acid. The reaction was stirred at RT and monitored by LCMS. Upon completion, the reaction was concentrated to afford a viscous oil. The crude material was purified by reverse phase chromatography (loading as a solution in ˜6 mL of 10 mM aqueous ammonium bicarbonate in H₂O adjusted to pH 10 with ammonia, C18 100 g Gold column, 60 mL/min, gradient 5-50% 10 mM aqueous ammonium bicarbonate in H₂O adjusted to pH 10 with ammonia to MeCN over 23 min) to afford the title compound (1.3 g, 1.351 mmol, 83% yield) as a colorless oil. LC-MS m/z 820.1 (M+H)⁺.

### Intermediate 5: 1-((2S,3S)-1-Methyl-5-oxo-2-(pyridin-3-yl)pyrrolidin-3-yl)-1-oxo-5,8,11,14,17,20,23,26,29,32,35,38,41,44,47,50,53,56,59,62-icosaoxa-2-azapentahexacontan-65-oic acid hydrochloride

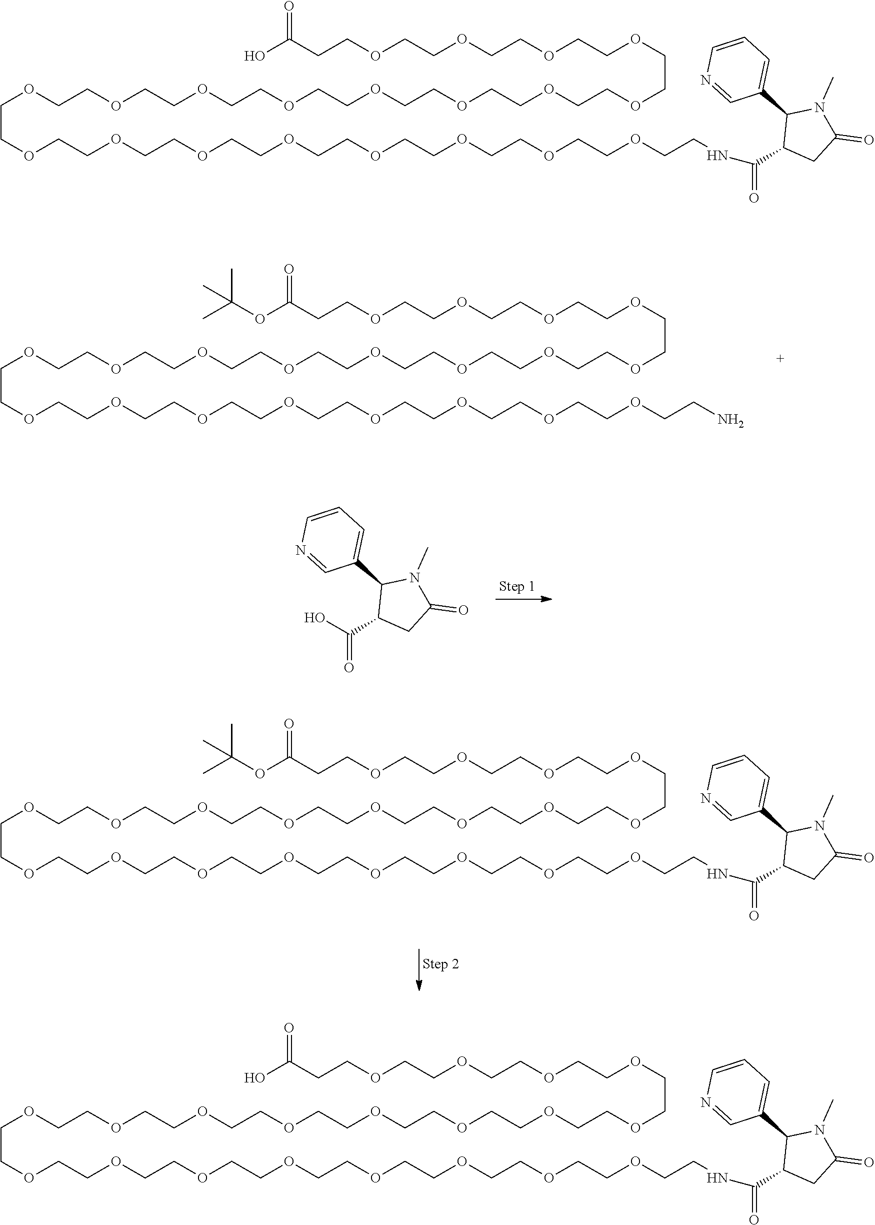 [SMILES: CN1C(=O)C[C@H](C(=O)NCCOCCOCCOCCOCCOCCOCCOCCOCCOCCOCCOCCOCCOCCOCCOCCOCCOCCOCCOCCOCCC(=O)O)[C@H]1c1cccnc1 ; CC(C)(C)OC(=O)CCOCCOCCOCCOCCOCCOCCOCCOCCOCCOCCOCCOCCOCCOCCOCCOCCOCCOCCOCCOCCN ; CN1C(=O)C[C@H](C(=O)O)[C@H]1c1cccnc1 ; CN1C(=O)C[C@H](C(=O)NCCOCCOCCOCCOCCOCCOCCOCCOCCOCCOCCOCCOCCOCCOCCOCCOCCOCCOCCOCCOCCC(=O)OC(C)(C)C)[C@H]1c1cccnc1 ; CN1C(=O)C[C@H](C(=O)NCCOCCOCCOCCOCCOCCOCCOCCOCCOCCOCCOCCOCCOCCOCCOCCOCCOCCOCCOCCOCCC(=O)O)[C@H]1c1cccnc1]

Step 1: tert-Butyl 1-((2S,3S)-1-methyl-5-oxo-2-(pyridin-3-yl)pyrrolidin-3-yl)-1-oxo-5,8,11,14,17,20,23,26,29,32,35,38,41,44,47,50,53,56,59,62-icosaoxa-2-azapentahexacontan-65-oate

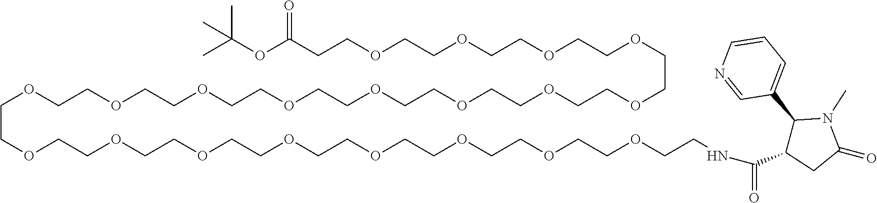 [SMILES: CN1C(=O)C[C@H](C(=O)NCCOCCOCCOCCOCCOCCOCCOCCOCCOCCOCCOCCOCCOCCOCCOCCOCCOCCOCCOCCOCCC(=O)OC(C)(C)C)[C@H]1c1cccnc1]

To a solution of (intermediate 1) (2S,3S)-1-methyl-5-oxo-2-(pyridin-3-yl)pyrrolidine-3-carboxylic acid (33.8 mg, 0.153 mmol) in DCM (1.5 mL) was added tert-butyl 1-((2S,3S)-1-methyl-5-oxo-2-(pyridin-3-yl)pyrrolidin-3-yl)-1-oxo-5,8,11,14,17,20,23,26,29,32,35,38,41,44,47,50,53,56,59,62-icosaoxa-2-azapentahexacontan-65-oate (131.8 mg, 0.086 mmol, 58.7% yield), HATU (66.7 mg, 0.175 mmol), and DI PEA (77 μl, 0.438 mmol). The mixture was stirred at RT for 18 h, then purified directly via ISCO CombiFlash@chromatography eluting with a gradient of 0 to 10% methanol in dichloromethane to provide the title compound as a yellow oil (131.8 mg, 0.107 mmol, 73.4% yield). LCMS m/z 1228.6 (M+H)⁺.

### Step 2: 1-((2S,3S)-1-Methyl-5-oxo-2-(pyridin-3-yl)pyrrolidin-3-yl)-1-oxo-5,8,11,14,17,20,23,26,29,32,35,38,41,44,47,50,53,56,59,62-icosaoxa-2-azapentahexacontan-65-oic acid hydrorhloride

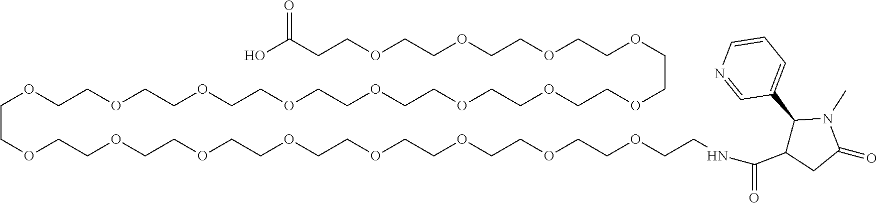 [SMILES: CN1C(=O)CC(C(=O)NCCOCCOCCOCCOCCOCCOCCOCCOCCOCCOCCOCCOCCOCCOCCOCCOCCOCCOCCOCCOCCC(=O)O)[C@H]1c1cccnc1]

To a solution of tert-butyl 1-((2S,3S)-1-methyl-5-oxo-2-(pyridin-3-yl)pyrrolidin-3-yl)-1-oxo-5,8,11,14,17,20,23,26,29,32,35,38,41,44,47,50,53,56,59,62-icosaoxa-2-azapentahexacontan-65-oate (127 mg, 0.103 mmol) in Dichloromethane (1.0 mL) was added 4M HCl in dioxane (31.4 μl, 1.034 mmol). The mixture was stirred at RT for 24 h. Additional 4M HCl in dioxane was added (31.4 μl, 1.03 mmol) and the mixture was stirred at RT for 24 h. The reaction was concentrated in vacuo to provide the crude title compound as a yellow oil (170 mg, 0.141 mmol, >theoretical yield) that was used without further purification. LCMS m/z 1172.3 (M+H)⁺.

The following compounds were or could be prepared with procedures analogous to that described in Intermediate 5

| Int. | Compound | LC-MS m/z |
|---|---|---|
| 6 | 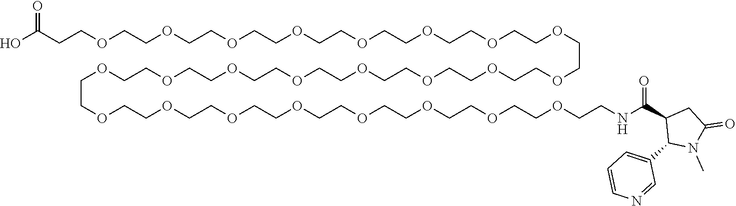 [SMILES: CN1C(=O)C[C@H](C(=O)NCCOCCOCCOCCOCCOCCOCCOCCOCCOCCOCCOCCOCCOCCOCCOCCOCCOCCOCCOCCOCCOCCOCCOCCOCCC(=O)O)[C@H]1c1cccnc1] | 690.6 (M + 2NH₄)/2 |
| | 1-((2S,3S)-1-methyl-5-oxo-2-(pyridin-3-yl)pyrrolidin-3-yl)-1-oxo- | |
| | 5,8,11,14,17,20,23,26,29,32,35,38,41,44,47,50,53,56,59,62,65,68,71,74- | |
| | tetracosaoxa-2-azaheptaheptacontan-77-oic acid | |

### Intermediate 7: (S)-6-((2-aminoethyl)(methyl)amino)-N-(2-(2-cyano-4,4-difluoropyrrolidin-1-yl)-2-oxoethyl)quinoline-4-carboxamide

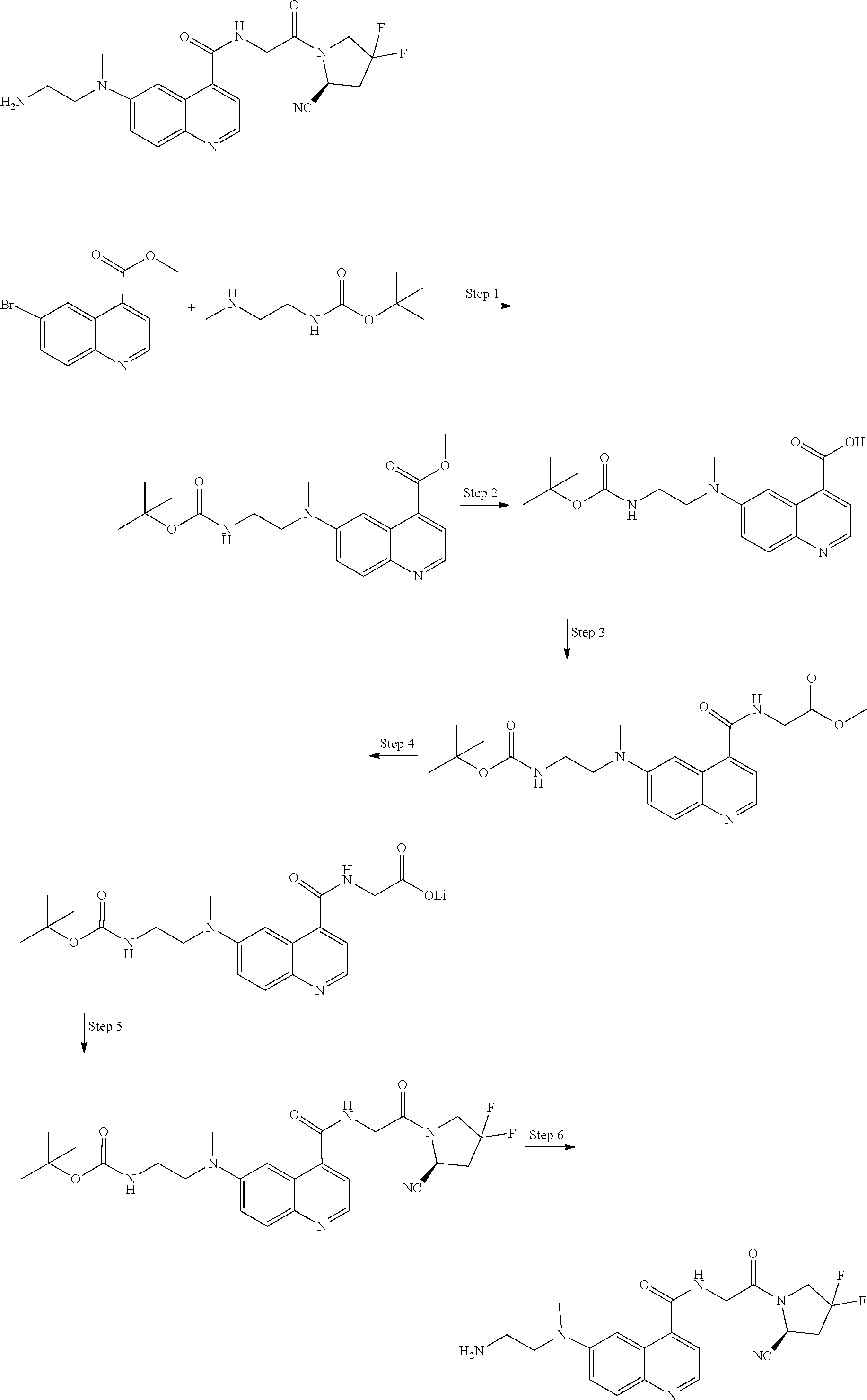

### Step 1: methyl 6-((2-((tert-butoxycarbonyl)amino)ethyl)(methyl)amino)quinoline-4-carboxylate

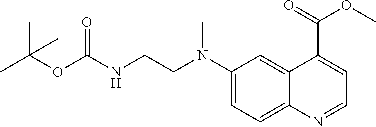 [SMILES: COC(=O)c1ccnc2ccc(N(C)CCNC(=O)OC(C)(C)C)cc12]

To a 25 mL microwave vial was added 2′-(dicyclohexylphosphaneyl)-N,N-dimethyl-[1,1′-biphenyl]-2-amine (37.0 mg, 0.094 mmol), Pd₂(dba)₃ (86 mg, 0.094 mmol) methyl 6-bromoquinoline-4-carboxylate (500 mg, 1.879 mmol), and potassium phosphate, dibasic (818 mg, 4.70 mmol). The mixture was placed under an atmosphere of nitrogen gas. Degassed 1,2-Dimethoxyethane (9.4 mL) was added followed by tert-butyl (2-(methylamino)ethyl)carbamate (517 μl, 5.64 mmol). The mixture was capped and heated to 90° C. for 3 nights. Added additional Pd₂(dba)₃ (86 mg, 0.094 mmol) and 2′-(dicyclohexylphosphaneyl)-N,N-dimethyl-[1,1′-biphenyl]-2-amine (37.0 mg, 0.094 mmol) were added, and the mixture was stirred at 90° C. for 3 nights. Additional 2′-(dicyclohexylphosphaneyl)-N,N-dimethyl-[1,1′-biphenyl]-2-amine (37.0 mg, 0.094 mmol), Pd₂(dba)₃ (86 mg, 0.094 mmol), and tert-butyl (2-(methylamino)ethyl)carbamate (517 μl, 5.64 mmol) were added under nitrogen gas and the mixture was heated another 24 h at 90° C., then cooled to RT. The reaction was filtered and concentrated in vacuo. To a solution of the resultant residue in DMF (5 mL) was added iodomethane (176 μl, 2.82 mmol) and K₂CO₃ (390 mg, 2.82 mmol). The mixture was stirred at RT for 20 h. Additional iodomethane (0.088 mL, 1.41 mmol) and K₂CO₃ (195 mg, 1.41 mmol) were added and let continue at RT for 27 h. Additional iodomethane (0.088 mL, 1.41 mmol) and K₂CO₃ (195 mg, 1.41 mmol) were added and stirred at RT for 24 h. The reaction was then heated to 40° C. for 1 h, then at 60° C. for 3 h. The reaction was diluted with EtOAc (50 mL) and water (30 mL). The insoluble particulates were removed via filtration, and the filtrate was separated. The organic fraction was washed with saturated aqueous sodium bicarbonate (3×30 mL) and brine (30 mL), dried over anhydrous sodium sulfate, filtered, and concentrated in vacuo. The resultant residue was purified by ISCO CombiFlash® chromatography eluting with a gradient of 0 to 60% ethyl acetate in hexanes to provide the title compound as an orange solid (225.9 mg, 0.628 mmol, 33.4% yield). LC-MS m/z 360.4 (M+H)⁺.

### Step 2: 6-((2-((tert-Butoxycarbonyl)amino)ethyl)(methyl)amino)quinoline-4-carboxylic acid

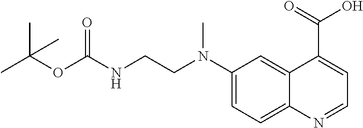 [SMILES: CN(CCNC(=O)OC(C)(C)C)c1ccc2nccc(C(=O)O)c2c1]

To a solution of methyl 6-((2-((tert-butoxycarbonyl)amino)ethyl)(methyl)amino)quinoline-4-carboxylate (720 mg, 2.003 mmol) in Methanol (10 mL) was added 0.5 M aqueous LiOH (4.8 mL, 2.4 mmol). The reaction was stirred at RT overnight then concentrated in vacuo, dried on high vac providing the title compound (692 mg, 1.983 mmol, 100% yield) as a yellow solid used without further purification. LC-MS m/z 346.1 (M+H)+

### Step 3: Methyl (6-((2-((tert-butoxycarbonyl)amino)ethyl)(methyl)amino)quinoline-4-carbonyl)glycinate

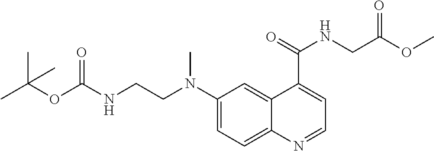 [SMILES: COC(=O)CNC(=O)c1ccnc2ccc(N(C)CCNC(=O)OC(C)(C)C)cc12]

To a solution of 6-((2-((tert-butoxycarbonyl)amino)ethyl)(methyl)amino)quinoline-4-carboxylic acid (1.400 g, 4.05 mmol) and methyl glycinate hydrochloride (0.524 g, 4.17 mmol) in N,N-Dimethylformamide (DMF) (13.51 ml) was added HATU (2.004 g, 5.27 mmol), and DIPEA (1.770 ml, 10.13 mmol). The mixture was stirred at RT overnight (18 h) then diluted with 100 mL EtOAc and washed with sat NaHCO₃ (3×50 mL) and brine (50 mL). Dried over sodium sulfate, filtered, and concentrated. Purified on 80 g ISCO gold silica gel column, eluting with 0-20% MeOH/DCM, resulting in the title compound (1.755g, >theoretical yield) as an orange-red solid.

LC-MS m/z 417.4 (M+H)+

### Step 4: Lithium (6-((2-((tert-butoxycarbonyl)amino)ethyl)(methyl)amino)quinoline-4-carbonyl)glycinate

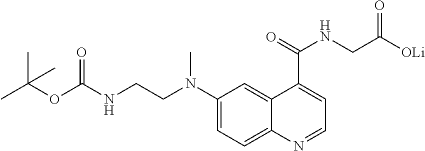

To a solution of methyl (6-((2-((tert-butoxycarbonyl)amino)ethyl)(methyl)amino)quinoline-4-carbonyl)glycinate (1.7 g, 4.08 mmol) in methanol (20.41 ml) was added 0.5 M aqueous LiOH (4.90 ml, 4.90 mmol). The reaction was stirred at RT overnight then concentrated in vacuo and dried on high vac, to afford the title compound (assumed theoretical yield) as a yellow solid (1.67 g, 4.04 mmol, 100% yield). LC-MS m/z 403.4 (M+H)⁺

### Step 5: tert-Butyl (S)-(2-((4-((2-(2-cyano-4,4-difluoropyrrolidin-1-yl)-2-oxoethyl) carbamoyl)quinolin-6-yl)(methyl)amino)ethyl)carbamate

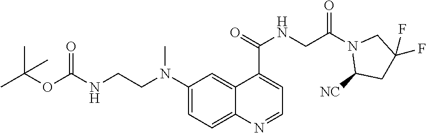 [SMILES: CN(CCNC(=O)OC(C)(C)C)c1ccc2nccc(C(=O)NCC(=O)N3CC(F)(F)C[C@H]3C#N)c2c1]

To a solution of lithium (6-((2-((tert-butoxycarbonyl)amino)ethyl)(methyl)amino)quinoline-4-carbonyl)glycinate (800 mg, 1.959 mmol) and (S)-4,4-difluoropyrrolidine-2-carbonitrile hydrochloride (363 mg, 2.155 mmol) in N,N-Dimethylformamide (DMF) (9.8 mL) was added HATU (968 mg, 2.55 mmol), and DIPEA (0.9 mL, 4.9 mmol). The mixture was stirred at RT overnight. The reaction was diluted with 50 mL EtOAc and washed with sat NaHCO₃ (3×30 mL), brine (30 mL), dried over sodium sulfate, filtered, and concentrated. Purified on 40 g ISCO gold silica gel column, eluting with 0-10% MeOH/DCM. The desired fractions were concentrated resulting in the title compound (296 mg, 29.3% yield). LC-MS m/z 517.1 (M+H)⁺

### Step 6: (S)-6-((2-aminoethyl)(methyl)amino)-N-(2-(2-cyano-4,4-difluoropyrrolidin-1-yl)-2-oxoethyl)quinoline-4-carboxamide

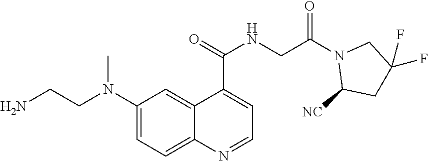 [SMILES: CN(CCN)c1ccc2nccc(C(=O)NCC(=O)N3CC(F)(F)C[C@H]3C#N)c2c1]

To a solution of tert-butyl (S)-(2-((4-((2-(2-cyano-4,4-difluoropyrrolidin-1-yl)-2-oxoethyl)carbamoyl)quinolin-6-yl)(methyl)amino)ethyl)carbamate (295 mg, 0.571 mmol) in Dichloromethane (DCM) (5.7 mL) was added TFA (0.44 mL, 5.71 mmol). The mixture was stirred at RT for 1.5 h then concentrated resulting in the title compound (487 mg, >theoretical yield) as a red solid. LC-MS m/z 417.1 (M+H)⁺

### Intermediate 8:6-((2-((1r,4r)-4-aminocyclohexane-1-carboxamido)ethyl)(methyl)amino)-N-(2-((S)-2-cyano-4,4-difluoropyrrolidin-1-yl)-2-oxoethyl)quinoline-4-carboxamide

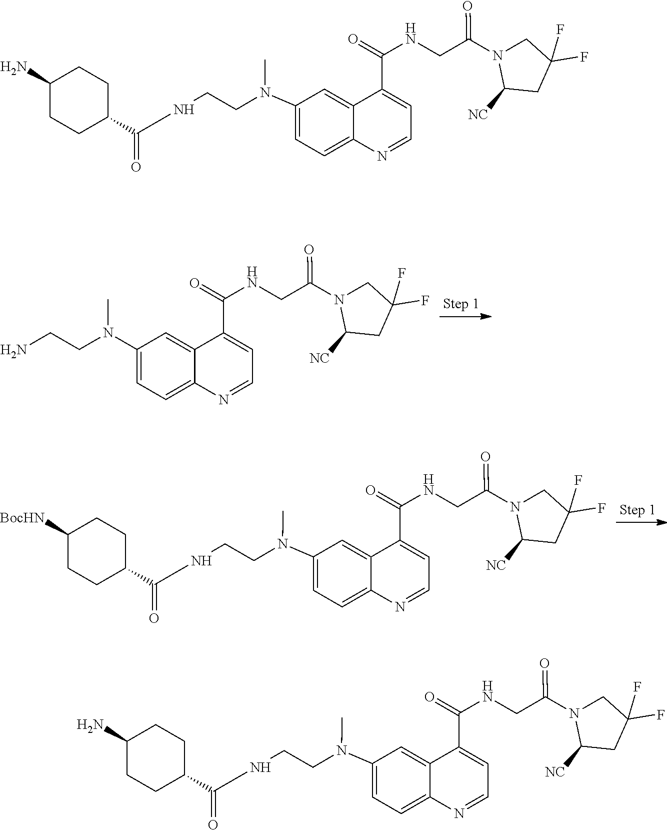 [SMILES: CN(CCNC(=O)[C@H]1CC[C@H](N)CC1)c1ccc2nccc(C(=O)NCC(=O)N3CC(F)(F)C[C@H]3C#N)c2c1 ; CN(CCN)c1ccc2nccc(C(=O)NCC(=O)N3CC(F)(F)C[C@H]3C#N)c2c1 ; CN(CCNC(=O)[C@H]1CC[C@H](NC(=O)OC(C)(C)C)CC1)c1ccc2nccc(C(=O)NCC(=O)N3CC(F)(F)C[C@H]3C#N)c2c1 ; CN(CCNC(=O)[C@H]1CC[C@H](N)CC1)c1ccc2nccc(C(=O)NCC(=O)N3CC(F)(F)C[C@H]3C#N)c2c1]

### Step 1: tert-Butyl ((1r,4r)-4-((2-((4-((2-((S)-2-cyano-4,4-difluoropyrrolidin-1-yl)-2-oxoethyl)carbamoyl)quinolin-6-yl)(methyl)amino)ethyl)carbamoyl)cyclohexyl)carbamate

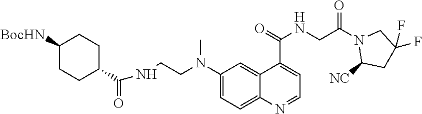 [SMILES: CN(CCNC(=O)[C@H]1CC[C@H](NC(=O)OC(C)(C)C)CC1)c1ccc2nccc(C(=O)NCC(=O)N3CC(F)(F)C[C@H]3C#N)c2c1]

To a solution of (S)-6-((2-aminoethyl)(methyl)amino)-N-(2-(2-cyano-4,4-difluoropyrrolidin-1-yl)-2-oxoethyl)quinoline-4-carboxamide (Intermediate 7) (64 mg, 0.154 mmol) in N,N-dimethylformamide (DMF) (1537 μl) was added (1r,4r)-4-((tert-butoxycarbonyl)amino)cyclohexane-1-carboxylic acid (56.1 mg, 0.231 mmol), HATU (88 mg, 0.231 mmol), and N,N-diisopropylethylamine (53.7 μl, 0.307 mmol). The reaction was stirred at RT overnight then diluted with EtOAc (50 mL), washed with saturated NaHCO₃ (2×30 mL), and brine (30 mL) then dried over sodium sulfate, filtered, and concentrated. Purified on 12 g ISCO gold silica gel column eluting with 0-10% MeOH/DCM. The desired fractions were concentrated resulting in the title compound (69.7 mg, 70.7% yield) as a yellow solid. LC-MS m/z 642.2 (M+H)⁺

### Step 2: 6-((2-((1r,4r)-4-Aminocyclohexane-1-carboxamido)ethyl)(methyl)amino)-N-(2-((S)-2-cyano-4,4-difluoropyrrolidin-1-yl)-2-oxoethyl)quinoline-4-carboxamide

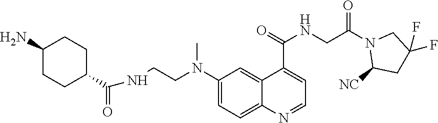 [SMILES: CN(CCNC(=O)[C@H]1CC[C@H](N)CC1)c1ccc2nccc(C(=O)NCC(=O)N3CC(F)(F)C[C@H]3C#N)c2c1]

To a solution of tert-butyl ((1r,4r)-4-((2-((4-((2-((S)-2-cyano-4,4-difluoropyrrolidin-1-yl)-2-oxoethyl)carbamoyl)quinolin-6-yl)(methyl)amino)ethyl)carbamoyl)cyclohexyl)carbamate (69 mg, 0.108 mmol) in Dichloromethane (DCM) (2150 μl) was added TFA (249 μl, 3.23 mmol). The reaction was stirred at RT for 1.5 h then concentrated and dried on high vac giving the title compound (61.9 mg, >theoretical yield) as a yellow solid. LC-MS m/z 542.2 (M+H)⁺

### Example 1: N-(2-((S)-2-Cyano-4,4-difluoropyrrolidin-1-yl)-2-oxoethyl)-6-(methyl(2-((1R,4R)-4-(4-(((1R,4R)-4-(2-((1R,4R)-4-(4-(((1 S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxamido)ethoxy)cyclohexyl)oxy)butanamido)cyclohexane-1-carboxamido)ethyl)amino) quinoline-4-carboxamide

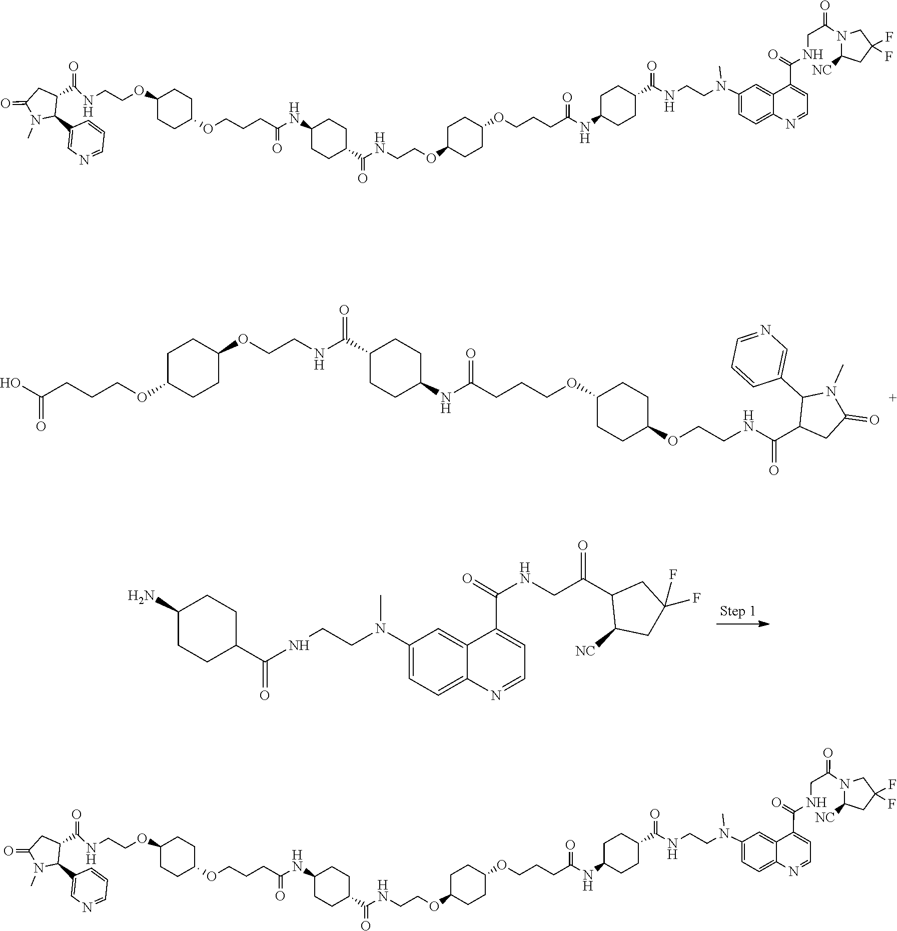

### Step 1: N-(2-((S)-2-Cyano-4,4-difluoropyrrolidin-1-yl)-2-oxoethyl)-6-(methyl (2-((1R,4R)-4-(4-(((1R,4R)-4-(2-((1R,4R)-4-(4-(((1 S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxamido) ethoxy)cyclohexyl)oxy)butanamido)cyclohexane-1-carboxamido)ethyl)amino)quinoline-4-carboxamide

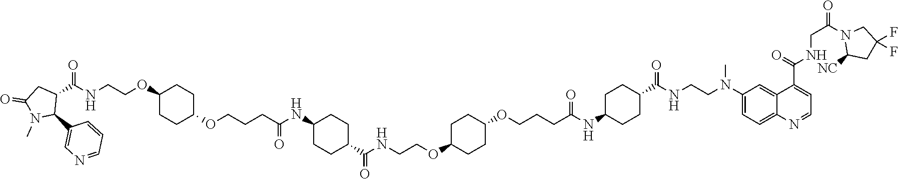 [SMILES: CN(CCNC(=O)[C@H]1CC[C@H](NC(=O)CCCO[C@H]2CC[C@H](OCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)CC1)c1ccc2nccc(C(=O)NCC(=O)N3CC(F)(F)C[C@H]3C#N)c2c1]

carboxamido)ethoxy)cyclohexyl)oxy)butanoic acid (Intermediate 3) (66.0 mg, 0.0825 mmol) in DMF (2.25 ml) was added 6-((2-((1R,4R)-4-aminocyclohexane-1-carboxamido)ethyl) (methyl)amino)-N-(2-((S)-2-cyano-4,4-difluoropyrrolidin-1-yl)-2-oxoethyl)quinoline-4-carboxamide (Intermediate 8) (61 mg, 0.113 mmol), HATU (55.7 mg, 0.146 mmol) and DIPEA (59.0 μl, 0.338 mmol). The reaction was stirred at RT for 21 h, then filtered with a syringe filter. The filtrate was purified via MDAP (XSelect™ CSH 018 column, 40 mU/min) eluting with a gradient of 15 to 55% acetonitrile in water containing formic acid (0.1%) to provide the title compound as a yellow-orange solid (12.9 mg, 0.00975 mmol, 8.65% yield). LMS m/z 662.6 (M+2H)⁺8 MH NMR (400 MHz, METHANOL-d₄) b ppm 1.05-1.46 (m, 16H) 1.52-1.71 (m, 4H) 1.75-2.05 (m, 20H) 2.12-2.29 (m, 5H) 2.66-2.67 (m, 3H) 2.68-2.74 (m, 1H) 2.78-3.02 (m, 4H) 3.03-3.10 (in, 2H) 3.13-3.15 (m, 3H) 3.23-3.30 (m, 5H) 3.43-3.56 (m, 10H) 3.60-3.76 (m, 3H) 4.09-4.35 (m, 3H) 4.82-4.85 (m, 1H) 5.11-5.21 (m, 1H) 7.46-7.49 (m, 1H) 7.51-7.53 (m, 1H) 7.53-7.55 (m, 1H) 7.55-7.58 (m, 1H) 7.75-7.87 (m, 4H) 7.87-7.93 (m, 1H) 7.88-7.91 (m, 1H) 8.50-8.53 (m, 1H) 8.53-8.57 (m, 1H) 8.57-8.62 (in, 1H).

The following compounds were or could be prepared with procedures analogous to that described in Example 1

| | Int. | |
|---|---|---|
| Ex. | Used | Compound |
| 2 | 82 | 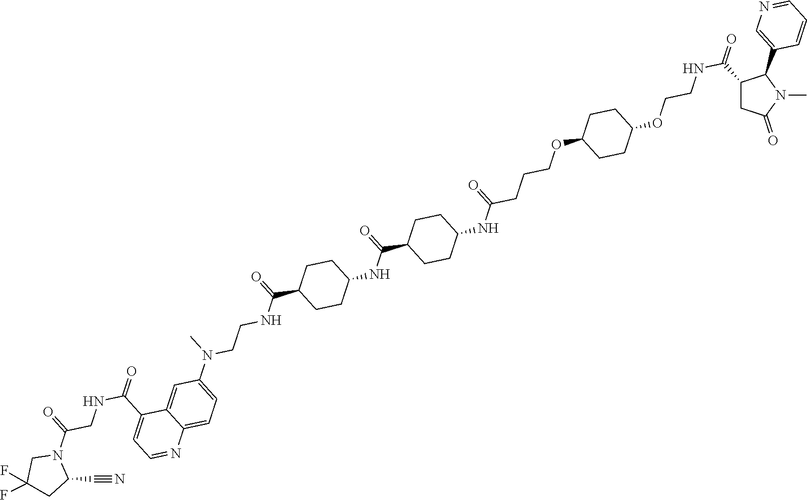 [SMILES: CN(CCNC(=O)[C@H]1CC[C@H](NC(=O)[C@H]2CC[C@H](NC(=O)CCCO[C@H]3CC[C@H](OCCNC(=O)[C@H]4CC(=O)N(C)[C@@H]4c4cccnc4)CC3)CC2)CC1)c1ccc2nccc(C(=O)NCC(=O)N3CC(F)(F)C[C@H]3C#N)c2c1] |
| | | N-(2-((S)-2-cyano-4,4-difluoropyrrolidin-1-yl)-2- |
| | | oxoethyl)-6-(methyl(2-((1R,4r)-((1R,4R)-4-(4- |
| | | (((1S,4R)-4-(2-((2S,3S)-1-methyl-5-oxo-2-(pyridin-3- |
| | | yl)pyrrolidine-3-carboxamido)ethoxy)cyclohexyl)oxy) |
| | | butanamido)cyclohexane-1-carboxamido) |
| | | cyclohexane-1-carboxamido)ethyl)amino)quinoline-4- |
| | | carboxamide |
| 3 | 3 | 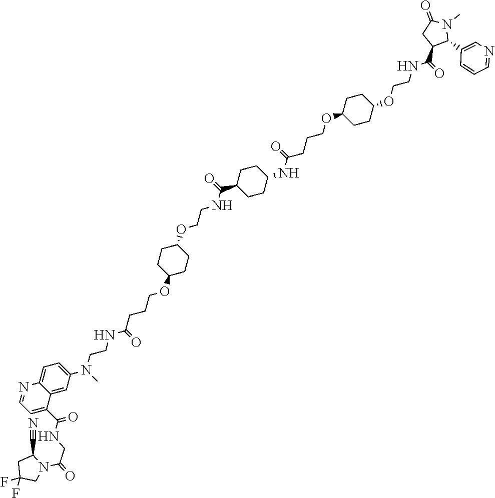 [SMILES: CN(CCNC(=O)CCCO[C@H]1CC[C@H](OCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)CC1)c1ccc2nccc(C(=O)NCC(=O)N3CC(F)(F)C[C@H]3C#N)c2c1] |
| | | N-(2-((S)-2-cyano-4,4-difluoropyrrolidin-1-yl)-2- |
| | | oxoethyl)-6-(methyl(2-(4-(((1R,4r)-4-(2-((1R,4R)-4-(4- |
| | | (((1S,4R)-4-(2-((2S,3S)-1-methyl-5-oxo-2-(pyridin-3- |
| | | yl)pyrrolidine-3-carboxamido)ethoxy)cyclohexyl)oxy) |
| | | butanamido)cyclohexane-1-carboxamido)ethoxy) |
| | | cyclohexyl)oxy)butanamido)ethyl)amino)quinoline-4- |
| | | carboxamide |
| 4 | 2 | 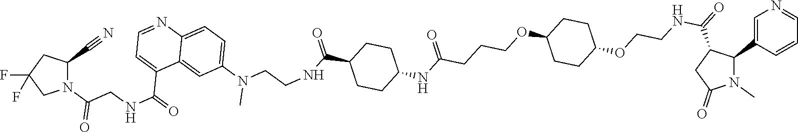 [SMILES: CN(CCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)c1ccc2nccc(C(=O)NCC(=O)N3CC(F)(F)C[C@H]3C#N)c2c1] |
| | | N-(2-((S)-2-cyano-4,4-difluoropyrrolidin-1-yl)-2- |
| | | oxoethyl)-6-(methyl(2-((1R,4r)-4-(4-(((1S,4R)-4-(2- |
| | | ((2S,3S)-1-methyl-5-oxo-2-(pyridin-3-yl)pyrrolidine-3- |
| | | carboxamido)ethoxy)cyclohexyl)oxy)butanamido) |
| | | cyclohexane-1-carboxamido)ethyl)amino)quinoline-4- |
| | | carboxamide |
| 5 | 4 | 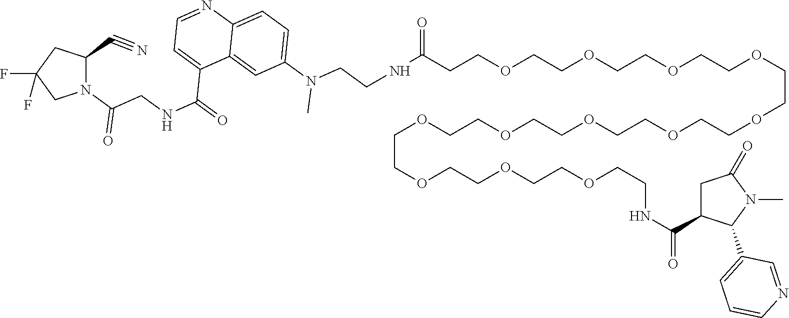 [SMILES: CN(CCNC(=O)CCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| | | N-(2-((S)-2-cyano-4,4-difluoropyrrolidin-1-yl)-2- |
| | | oxoethyl)-6-(methyl(1-((2S,3S)-1-methyl-5-oxo-2- |
| | | (pyridin-3-yl)pyrrolidin-3-yl)-1,41-dioxo- |
| | | 5,8,11,14,17,20,23,26,29,32,35,38-dodecaoxa-2,42- |
| | | diazatetratetracontan-44-yl)amino)quinoline-4- |
| | | carboxamide |
| 6 | 5 | 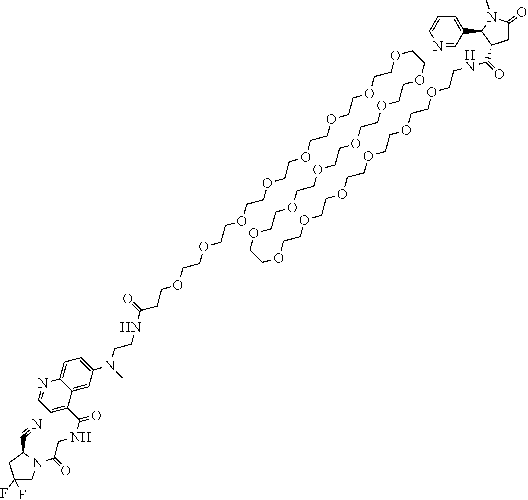 [SMILES: CN(CCNC(=O)CCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| | | N-(2-((S)-2-cyano-4,4-difluoropyrrolidin-1-yl)-2- |
| | | oxoethyl)-6-(methyl(1-((2S,3S)-1-methyl-5-oxo-2- |
| | | (pyridin-3-yl)pyrrolidin-3-yl)-1,65-dioxo-5,8,11,14,17, |
| | | 20,23,26,29,32,35,38,41,44,47,50,53,56,59,62- |
| | | icosaoxa-2,66-diazaoctahexacontan-68- |
| | | yl)amino)quinoline-4-carboxamide |
| 7 | 6 | 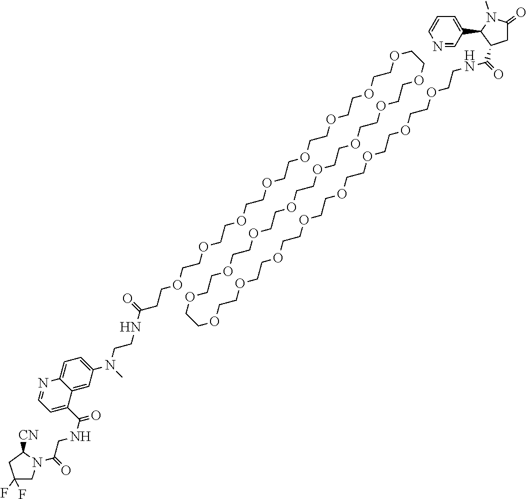 [SMILES: CN(CCNC(=O)CCOCCOCCOCCOCCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| | | N-(2-((S)-2-Cyano-4,4-difluoropyrrolidin-1-yl)-2- |
| | | oxoethyl)-6-(methyl(1-((2S,3S)-1-methyl-5-oxo-2- |
| | | (pyridin-3-yl)pyrrolidin-3-yl)-1,77-dioxo-5,8,11,14,17, |
| | | 20,23,26,29,32,35,38,41,44,47,50,53,56,59,62,65,68,71, |
| | | 74-tetracosaoxa-2,78-diazaoctacontan-80- |
| | | yl)amino)quinoline-4-carboxamide |

| Ex. | LC-MS m/z | NMR |
|---|---|---|
| 2 | M + H: 1096.3 | ¹H NMR (400 MHz, METHANOL-d₄) δ ppm 1.05-1.32 (m, 8 H) 1.33-1.46 (m, 2 H) 1.48-1.70 (m, 4 H) 1.76-1.86 (m, 6 H) |
| | | 1.88-2.03 (m, 6 H) 2.20-2.28 (m, 2 H) 2.66-2.67 (m, 3 H) 2.70-2.74 (m, 1 H) 2.78-2.88 (m, 3 H) 2.89-3.10 (m, 3 H) 3.02- |
| | | 3.10 (m, 2 H) 3.13-3.15 (m, 3 H) 3.23-3.31 (m, 5 H) 3.41-3.54 (m, 6 H) 3.67-3.74 (m, 2 H) 4.12-4.30 (m, 2 H) 4.30-4.34 |
| | | (m, 1 H) 4.82-4.85 (m, 1 H) 5.12-5.22 (m, 1 H) 7.45-7.49 (m, 1 H) 7.50-7.58 (m, 3 H) 7.62-7.67 (m, 1 H) 7.78-7.86 |
| | | (m, 2 H) 7.87-7.92 (m, 1 H) 8.46-8.49 (m, 1 H) 8.51-8.53 (m, 1 H) 8.53-8.56 (m, 1 H) 8.57-8.60 (m, 1 H) |
| 3 | M + H: 1199.4 | ¹H NMR (400 MHz, METHANOL-d₄) δ ppm 1.14-1.39 (m, 12 H) 1.50-1.64 (m, 2 H) 1.66-1.74 (m, 2 H) 1.78-2.04 (m, 17 H) |
| | | 2.10-2.19 (m, 2 H) 2.21-2.32 (m, 3 H) 2.62-2.75 (m, 4 H) 2.77-2.96 (m, 2 H) 2.97-3.11 (m, 2 H) 3.12-3.22 (m, 4 H) |
| | | 3.24-3.30 (m, 4 H) 3.42-3.55 (m, 9 H) 3.57-3.75 (m, 3 H) 4.11-4.39 (m, 3 H) 4.82-4.85 (m, 1 H) 5.13-5.21 (m, 1 H) 7.43-7.60 |
| | | (m, 3 H) 7.75-7.87 (m, 3 H) 7.88-7.93 (m, 1 H) 7.93-8.02 (m, 1 H) 8.48-8.62 (m, 3 H) |
| 4 | M + H: 971.3 | ¹H NMR (400 MHz, METHANOL-d₄) δ ppm 1.04-1.19 (m, 2 H) 1.21-1.33 (m, 4 H) 1.35-1.49 (m, 2 H) 1.61-1.70 (m, 2 H) |
| | | 1.75-1.86 (m, 4 H) 1.88-2.03 (m, 5 H) 2.16-2.28 (m, 2 H) 2.65-2.67 (m, 3 H) 2.68-2.74 (m, 1 H) 2.78-3.00 (m, 3 H) |
| | | 3.02-3.10 (m, 2 H) 3.13-3.17 (m, 3 H) 3.23-3.30 (m, 4 H) 3.36-3.42 (m, 1 H) 3.44-3.56 (m, 6 H) 3.65-3.75 (m, 2 H) 4.11- |
| | | 4.29 (m, 2 H) 4.30-4.36 (m, 1 H) 4.81-4.85 (m, 1 H) 5.10-5.21 (m, 1 H) 7.45-7.50 (m, 1 H) 7.50-7.58 (m, 3 H) 7.75-7.82 |
| | | (m, 2 H) 7.83-7.87 (m, 1 H) 7.87-7.92 (m, 1 H) 8.50-8.53 (m, 1 H) 8.53-8.56 (m, 1 H) 8.56-8.60 (m, 1 H) |
| 5 | M + H: 1219.3 | ¹H NMR (400 MHz, METHANOL-d₄) δ ppm 2.32-2.41 (m, 2 H) 2.63-2.69 (m, 4 H) 2.70-2.76 (m, 1 H) 2.78-2.99 (m, |
| | | 4 H) 3.01-3.11 (m, 2 H) 3.37-3.44 (m, 1 H) 3.45-3.67 (m, 53 H) 3.68-3.76 (m, 1 H) 4.11-4.43 (m, 3 H) 4.80-4.84 (m, 1 |
| | | H) 5.13-5.25 (m, 1 H) 7.46-7.49 (m, 1 H) 7.49-7.51 (m, 1 H) 7.51-7.61 (m, 2 H) 7.76-7.85 (m, 1 H) 7.89-7.94 (m, 1 |
| | | H) 7.96-8.03 (m, 1 H) 8.50-8.54 (m, 1 H) 8.54-8.61 (m, 2 H) |
| 6 | M + 2H/2 = | ¹H NMR (400 MHz, METHANOL-d₄) δ ppm 2.31-2.41 (m, 2 H) 2.55-2.64 (m, 1 H) 2.65-2.69 (m, 3 H) 2.69-2.76 (m, 1 H) |
| | 786.1 | 2.80-2.92 (m, 2 H) 3.00-3.11 (m, 1 H) 3.14-3.20 (m, 3 H) 3.37-3.45 (m, 1 H) 3.47-3.68 (m, 85 H) 3.68-3.84 (m, 3 H) |
| | | 4.13-4.41 (m, 3 H) 4.80-4.85 (m, 1 H) 5.14-5.24 (m, 1 H) 7.46-7.49 (m, 1 H), 7.50-7.52 (m, 1 H) 7.52-7.61 (m, 2 H) 7.79- |
| | | 7.84 (m, 1 H) 7.89-7.94 (m, 1 H) 8.52-8.54 (m, 1 H) 8.55-8.57 (m, 1 H) 8.58-8.60 (m, 1 H) |
| 7 | 874.1 | ¹H NMR (400 MHz, METHANOL-d₄) δ ppm 2.32-2.40 (m, 2 H) 2.56-2.63 (m, 1 H) 2.65-2.69 (m, 3 H) 2.71-2.78 (m, |
| | (M + 2H)⁺⁺ | 1 H) 2.80-2.91 (m, 2 H) 3.01-3.12 (m, 2 H) 3.14-3.20 (m, 3 H) 3.36-3.45 (m, 1 H) 3.47-3.67 (m, 100 H) 3.68-3.83 (m, 2 |
| | | H) 4.12-4.25 (m, 1 H) 4.26-4.41 (m, 2 H) 4.81-4.85 (m, 1 H) 5.15-5.22 (m, 1 H) 7.46-7.49 (m, 1 H) 7.50-7.52 (m, 1 H) 7.52- |
| | | 7.61 (m, 2 H) 7.79-7.85 (m, 1 H) 7.89-7.94 (m, 1 H) 8.52-8.55 (m, 1 H) 8.55-8.57 (m, 1 H) 8.58-8.60 (m, 1 H). |

### Example 8: N-(2-((R)-2-cyanopyrrolidin-1-yl)-2-oxoethyl)-6-((1-((2S,3S)-1-methyl-5-oxo-2-(pyridin-3-yl)pyrrolidin-3-yl)-1,51-dioxo-5,8,11,14,17,20,23,26,29,32,35,38,41,44,47-pentadecaoxa-2,50-diazadopentacontan-52-yl)oxy)quinoline-4-carboxamide

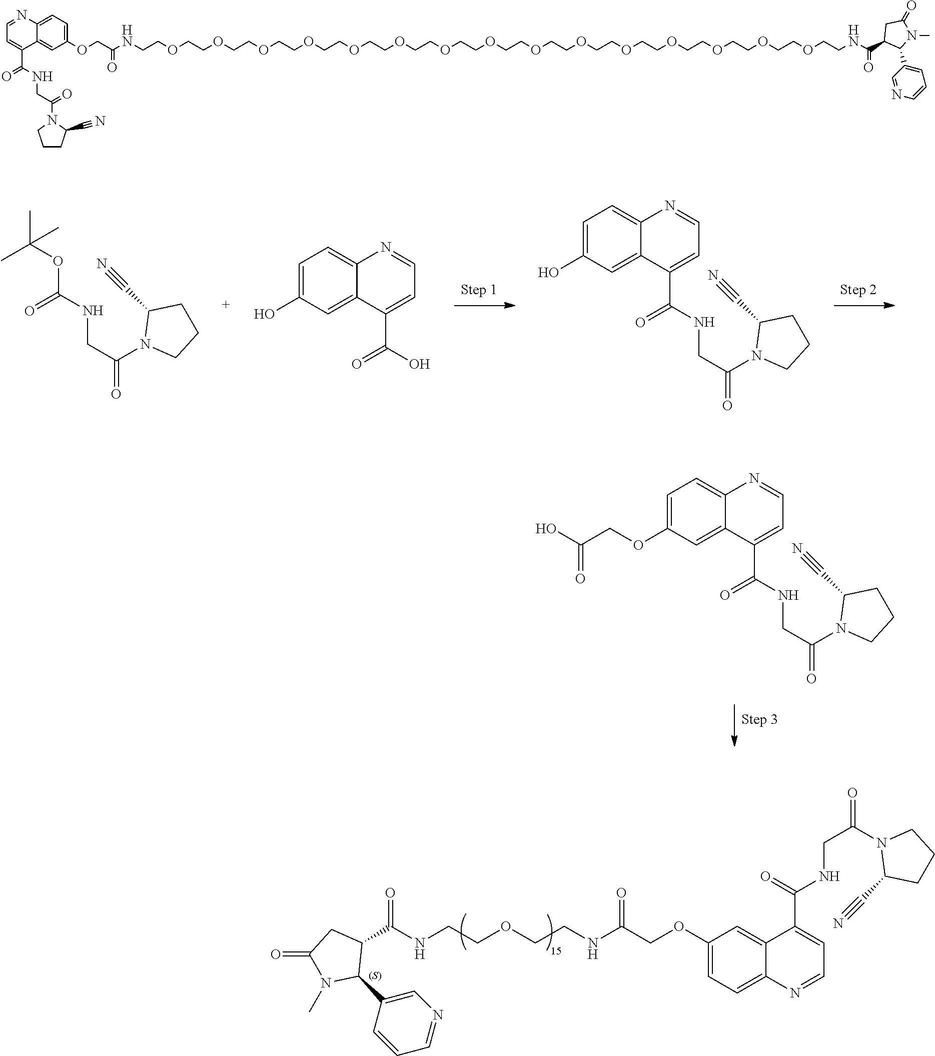

### Step 1: (S)—N-(2-(2-cyanopyrrolidin-1-yl)-2-oxoethyl)-6-hydroxyquinoline-4-carboxamide

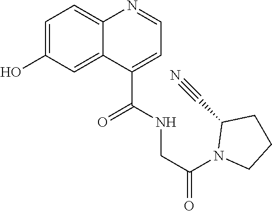 [SMILES: N#C[C@@H]1CCCN1C(=O)CNC(=O)c1ccnc2ccc(O)cc12]

tert-Butyl (S)-(2-(2-cyanopyrrolidin-1-yl)-2-oxoethyl)carbamate (500 mg, 1.974 mmol) was dissolved in Dichloromethane (DCM) (1 mL) and TFA (1 mL, 12.98 mmol). After 30 min, the reaction was concentrated under a stream of nitrogen at 40° C. and dried under high vacuum, resulting in an amber gum. 6-hydroxyquinoline-4-carboxylic acid (448 mg, 2.369 mmol) and HATU (901 mg, 2.369 mmol) were added followed by N,N-Dimethylformamide (DMF) (15 mL). Once the reaction became homogeneous TEA (1.651 mL, 11.84 mmol) was added. After 30 min, the reaction was diluted with water and purified on a C18 cartridge (100 g) with a Combiflash Rf+, eluting at 60 mL/min with a non-linear 10-100% (0.1% NH4OH/ACN)/(0.1% NH4OH/H2O) gradient. The desired fractions were concentrated under reduced pressure and dried under high vacuum, giving the title compound (132 mg, 0.407 mmol, 20.62% yield) as a yellow solid. LC-MS m/z 325.1 (M+H)⁺.

### Step 2: (S)-2-((4-((2-(2-cyanopyrrolidin-1-yl)-2-oxoethyl)carbamoyl)quinolin-6-yl)oxy) acetic acid

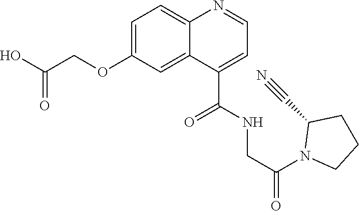 [SMILES: N#C[C@@H]1CCCN1C(=O)CNC(=O)c1ccnc2ccc(OCC(=O)O)cc12]

Potassium carbonate (201 mg, 1.452 mmol) was added to (S)—N-(2-(2-cyanopyrrolidin-1-yl)-2-oxoethyl)-6-hydroxyquinoline-4-carboxamide (157 mg, 0.484 mmol) and the mixture was suspended in acetone (3.7 mL). Ethyl 2-bromoacetate (0.065 mL, 0.581 mmol) was added, and the reaction was stirred at rt for 6 h, then water (2 mL) was added. After 36 h, the reaction was concentrated under a stream of nitrogen at 40° C. resulting in a red solid. The solid was dissolved in water and purified on a C18 cartridge (50 g) with a Combiflash Rf+, eluting at 40 mL/min with a non-linear 0-30% (0.1% NH4OH/ACN)/(0.1% NH4OH/H2O) gradient. The desired fractions were concentrated under reduced pressure and dried under high vacuum, giving the title compound (120 mg, 0.314 mmol, 64.8% yield) as a red solid. LC-MS m/z 383.3 (M+H)⁺.

### Step 3: N-(2-((R)-2-cyanopyrrolidin-1-yl)-2-oxoethyl)-6-((1-((2S,3S)-1-methyl-5-oxo-2-(pyridin-3-yl)pyrrolidin-3-yl)-1,51-dioxo-5,8,11,14,17,20,23,26,29,32,35,38,41,44,47-pentadecaoxa-2,50-diazadopentacontan-52-yl)oxy)quinoline-4-carboxamide

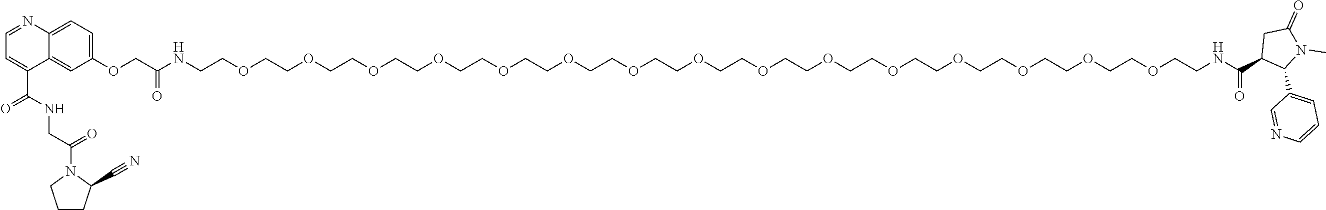 [SMILES: CN1C(=O)C[C@H](C(=O)NCCOCCOCCOCCOCCOCCOCCOCCOCCOCCOCCOCCOCCOCCOCCOCCNC(=O)COc2ccc3nccc(C(=O)NCC(=O)N4CCC[C@@H]4C#N)c3c2)[C@H]1c1cccnc1]

(S)-2-((4-((2-(2-cyanopyrrolidin-1-yl)-2-oxoethyl)carbamoyl)quinolin-6-yl)oxy)acetic acid (43 mg, 0.112 mmol) and TSTU (36.0 mg, 0.120 mmol) were dissolved in Dimethyl Sulfoxide (DMSO) (1 mL) and DIPEA (0.05 mL, 0.286 mmol). After 15 min (2S,3S)—N-(47-amino-3,6,9,12,15,18,21,24,27,30,33,36,39,42,45-pentadecaoxaheptatetracontyl)-1-methyl-5-oxo-2-(pyridin-3-yl)pyrrolidine-3-carboxamide (96 mg, 0.104 mmol) dissolved in Dimethyl Sulfoxide (DMSO) (1 mL) was added. After 1.5 h the reaction was quenched with water and the crude product was purified on a C18 cartridge (100 g) with a Combiflash Rf+, eluting at 60 mL/min with a non-linear 10-100% (0.1% NH4OH/ACN)/(0.1% NH4OH/H2O) gradient. The desired fractions were concentrated under reduced pressure and dried under high vacuum, giving the title compound (104 mg, 0.081 mmol, 78% yield) as a sticky gum. LC-MS m/z 644.7 (M+H)⁺, ¹H NMR (400 MHz, DEUTERIUM OXIDE) b ppm 2.08-2.29 (m, 3H) 2.50-2.59 (m, 3H) 2.62-2.82 (m, 2H) 2.98-3.06 (m, 1H) 3.29-3.66 (m, 64H) 3.67-3.74 (m, 1H) 4.21-4.36 (m, 2H) 4.57-4.79 (m, 5H) 7.40-7.47 (m, 3H) 7.54 (d, J=4.52 Hz, 1H) 7.68-7.71 (m, 1.88 Hz, 1H) 7.88 (d, J=9.29 Hz, 1H) 8.35-8.36 (m, 1H) 8.43-8.45 (m, 1H) 8.66 (d, J=4.50 Hz, 1H).

### Example 9: N-(2-((S)-2-cyanopyrrolidin-1-yl)-2-oxoethyl)-7-((1-((2S,3S)-1-methyl-5-oxo-2-(pyridin-3-yl)pyrrolidin-3-yl)-1-oxo-5,8,11,14,17,20,23,26,29,32,35,38,41,44,47-pentadecaoxa-2-azanonatetracontan-49-yl)oxy)quinoline-4-carboxamide

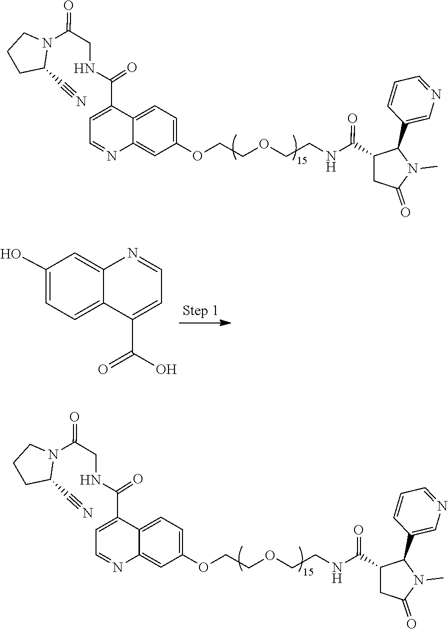

### Step 1: N-(2-((S)-2-cyanopyrrolidin-1-yl)-2-oxoethyl)-7-((1-((2S,3S)-1-methyl-5-oxo-2-(pyridin-3-yl)pyrrolidin-3-yl)-1-oxo-5,8,11,14,17,20,23,26,29,32,35,38,41,44,47-pentadecaoxa-2-azanonatetracontan-49-yl)oxy)quinoline-4-carboxamide

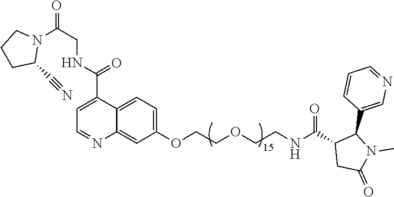

One pot multi-step reaction: 7-hydroxyquinoline-4-carboxylic acid (95 mg, 0.50 mmol) was suspended in Methanol (0.7 mL). Sulfuric acid (0.054 mL, 1.004 mmol) was added dropwise and the reaction immediately darkened and the solid went into solution. The reaction was heated for 18 h at 50° C. then loaded onto an SCX3 cartridge, washed with MeOH (three times), then eluted with NH₃ (2 N in MeOH) (three times), split into two equal portions and concentrated under reduced pressure. To one portion, triphenylphosphine (79 mg, 0.301 mmol), and tert-butyl (47-hydroxy-3,6,9,12,15,18,21,24,27,30,33,36,39,42,45-pentadecaoxaheptatetracontyl)carbamate (206 mg, 0.251 mmol) was added and dissolved in THF (8 mL). DEAD (40 wt % in Toluene) (0.12 mL, 0.263 mmol) was added, and the reaction was heated at 70° C. for 16 h. The reaction was loaded onto SCX-3 cartridges (2 g), washed with MeOH, then eluted with 2N NH3/MeOH. The eluant was concetrated resulting in a syrup. NaOH (1 N) (0.4 mL, 0.400 mmol) was added, followed by THF (0.7 mL), water (0.7 mL), MeOH (0.7 mL). This was stirred for 1.5 h then concentrated under a stream of nitrogen at 40° C. The resultant paste was taken up in HCl (4 M, dioxane) (1.8 mL, 7.20 mmol) and stirred for 1 h then concentrated under a stream of nitrogen at 40° C. The resultant gum was dissolved in DMSO (2 mL) and added to (2S,3S)-1-methyl-5-oxo-2-(pyridin-3-yl)pyrrolidine-3-carboxylic acid (47.0 mg, 0.213 mmol) that had previously been dissolved in DMSO with TSTU (68 mg, 0.23 mmol) and DIPEA (0.175 mL, 1.004 mmol) for 10 minutes. The reaction was stirred for 30 min, then TSTU (68.0 mg, 0.226 mmol) was added followed by (intermediate 8) (S)-1-glycylpyrrolidine-2-carbonitrile tetrakis(4-methylbenzenesulfonate) (211 mg, 0.251 mmol) and DIPEA (0.175 mL, 1.004 mmol) 15 minutes later. The final amide reaction was stirred for 1.5 h then loaded onto a C18 cartridge (100 g) with a Combiflash Rf+, eluting at 60 mL/min with a non-linear 10-100% (0.1% NH₄OH/ACN)/(0.1% NH₄OH/H₂O) gradient. The desired fractions were concentrated under reduced pressure and dried under high vacuum, giving the title compound (63 mg, 0.051 mmol, 10.20% yield) as a clear gum. LC-MS m/z 1230.8 (M+H)⁺, ¹H NMR (400 MHz, DEUTERIUM OXIDE) b ppm 2.00-2.31 (m, 4H) 2.52 (s, 3H) 2.61-2.71 (m, 1H) 2.73-2.83 (m, 1H) 2.96-3.06 (m, 1H) 3.11-3.24 (m, 1H) 3.26-3.76 (m, 62H) 3.87 (br s, 2H) 4.22 (br s, 4H) 7.16-7.26 (m, 2H) 7.35-7.47 (m, 2 H) 7.38-7.44 (m, 1H) 7.64-7.73 (m, 1H) 7.94-8.06 (m, 1H) 8.35 (s, 1H) 8.40-8.48 (m, 1H) 8.63-8.73 (m, 1H).

### Example 10 and 11: N-(2-((S)-2-cyanopyrrolidin-1-yl)-2-oxoethyl)-7-((1-((2S,3S)-1-methyl-5-oxo-2-(pyridin-3-yl)pyrrolidin-3-yl)-1,51-dioxo-5,8,11,14,17,20,23,26,29,32,35,38,41,44,47-pentadecaoxa-2,50-diazadopentacontan-52-yl)oxy)quinoline-4-carboxamide and N-(2-((S)-2-cyano-4,4-difluoropyrrolidin-1-yl)-2-oxoethyl)-7-((1-((2S,3S)-1-methyl-5-oxo-2-(pyridin-3-yl)pyrrolidin-3-yl)-1,51-dioxo-5,8,11,14,17,20,23,26,29,32,35,38,41,44,47-pentadecaoxa-2,50-diazadopentacontan-52-yl)oxy)quinoline-4-carboxamide

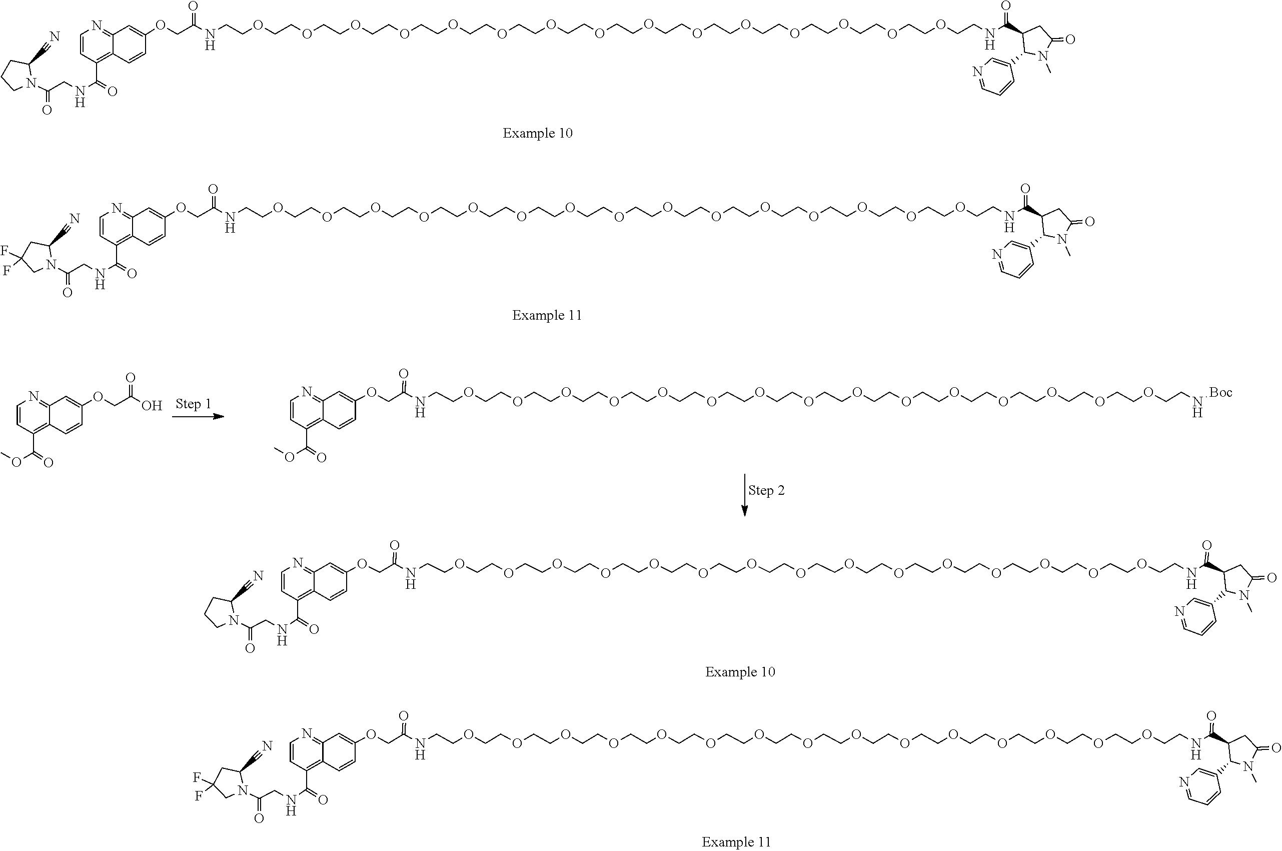

Step 1: methyl 7-((2,2-dimethyl-4,54-dioxo-3,8,11,14,17,20,23,26,29,32,35,38,41,44,47,50-hexadecaoxa-5,53-diazapentapentacontan-55-yl)oxy)quinoline-4-carboxylate tert-Butyl (47-amino-3,6,9,12,15,18,21,24,27,30,33,36,39,42,45-pentadecaoxaheptatetracontyl) carbamate (173 mg, 0.211 mmol) and 2-((4-(methoxycarbonyl)quinolin-7-yl)oxy)acetic acid (50 mg, 0.191 mmol) were dissolved in N,N-Dimethylformamide (DMF) (479 μl) and N-methylmorpholine (84 μl, 0.766 mmol). Once fully dissolved, HATU (87 mg, 0.230 mmol) dissolved in N,N-Dimethylformamide (DMF) (479 μl) was added and the reaction was stirred for 2 h then diluted with water and purified on a C18 cartridge (50 g) with a Combiflash Rf+, eluting at 40 mL/min with a non-linear 10-100% (0.1% NH₄OH/ACN)/(0.1% NH₄OH/H₂O) gradient. The desired fractions were concentrated under reduced pressure and dried under high vacuum, giving lot A1 of methyl 7-((2,2-dimethyl-4,54-dioxo-3,8,11,14,17,20,23,26,29,32,35,38,41,44,47,50-hexadecaoxa-5,53-diazapentapentacontan-55-yl)oxy)quinoline-4-carboxylate (120 mg, 0.113 mmol, 58.9% yield) as a clear gum. LC-MS m/2z 532.8 (M+2H)⁺⁺.

Step 2: N-(2-((S)-2-cyanopyrrolidin-1-yl)-2-oxoethyl)-7-((1-((2S,3S)-1-methyl-5-oxo-2-(pyridin-3-yl)pyrrolidin-3-yl)-1,51-dioxo-5,8,11,14,17,20,23,26,29,32,35,38,41,44,47-pentadecaoxa-2,50-diazadopentacontan-52-yl)oxy)quinoline-4-carboxamide and N-(2-((S)-2-cyano-4,4-difluoropyrrolidin-1-yl)-2-oxoethyl)-7-((1-((2S,3S)-1-methyl-5-oxo-2-(pyridin-3-yl)pyrrolidin-3-yl)-1,51-dioxo-5,8,11,14,17,20,23,26,29,32,35,38,41,44,47-pentadecaoxa-2,50-diazadopentacontan-52-yl)oxy)quinoline-4-carboxamide

One step multipot reaction: methyl 7-((2,2-dimethyl-4,54-dioxo-3,8,11,14,17,20,23,26,29,32,35,38,41,44,47,50-hexadecaoxa-5,53-diazapentapentacontan-55-yl)oxy)quinoline-4-carboxylate (120 mg, 0.113 mmol) was dissolved in NaOH (2 N) (0.11 mL, 0.220 mmol) and Acetonitrile (0.3 mL) and the reaction was heated to 40° C. (external) for 45 min, then concentrated under a stream of nitrogen at 40° C. The resultant residue was vigorously suspended in HCl (4 N in dioxane) (0.8 mL, 3.20 mmol). After 30 min the reaction was concentrated under a stream of nitrogen at 45° C., then suspended and reconcentrated with ACN (twice). The resultant residue was dissolved in Dimethyl Sulfoxide (DMSO) (1.1 mL) and 4-methylmorpholine (0.02 mL, 0.182 mmol) and added to a solution of (2S,3S)-1-methyl-5-oxo-2-(pyridin-3-yl)pyrrolidine-3-carboxylic acid (14.90 mg, 0.068 mmol) and TSTU (21 mg, 0.070 mmol) in Dimethyl Sulfoxide (DMSO) (1.1 mL) and 4-methylmorpholine (0.01 mL, 0.091 mmol) that had been stirring for 5 min. After 30 min of the amino acid intermediate reacting with the NHS ester, a second aliquot of TSTU (21 mg, 0.070 mmol) was added. After 5 min the reactions were split in half by volume and either (S)-1-glycylpyrrolidine-2-carbonitrile tetrakis(4-methylbenzenesulfonate) (29 mg, 0.034 mmol) [for example 10] or (S)-4,4-difluoro-1-glycylpyrrolidine-2-carbonitrile 4-methylbenzenesulfonate (13 mg, 0.036 mmol) [for example 11] was added. 4-methylmorpholine (0.02 mL, 0.182 mmol) was added to ensure basic pH. After 1 h, the reactions were diluted with water and purified on an open access MDAP [XSELECT CSH C18 column (150 mm×30 mm i.d. 5 μm packing diameter) at ambient temperature. The solvents employed were: A=10 mM Ammonium Bicarbonate in H₂O adjusted to pH 10 with Ammonia. B=Acetonitrile.]15-99% gradient B/A. The desired fractions were collected and concentrated under reduced pressure resulting in example 10 (11.4 mg, 8.85 μmol, 7.85% yield) and example 11 (9.5 mg, 7.18 μmol, 6.37% yield) as clear oils. Example 10: LC-MS m/z 1288.1 (M+H)⁺1H NMR (400 MHz, DEUTERIUM OXIDE) b ppm 2.39-2.42 (m, 3H) 2.53-2.71 (m, 3H) 2.99 (s, 3H) 3.03-3.12 (m, 1H) 3.17-3.26 (m, 1H) 3.40-3.47 (m, 1H) 3.63-3.77 (m, 2H) 3.82-4.06 (m, 62H) 4.10-4.15 (in, 1H) 4.67-4.76 (in, 1H) 5.14-5.19 (m, 4H) 7.83-7.92 (m, 4H) 8.14-8.17 (m, 1H) 8.65 (d, J=9.29 Hz, 1H) 8.84-8.86 (m, 1H) 8.93-8.95 (m, 1H) 9.26 (d, J=4.52 Hz, 1H) Example 11: LC-MS m/z 1324.2 (M+H)⁺, ¹H NMR (400 MHz, DEUTERIUM OXIDE) ppm 2.36-2.44 (i, 4H) 2.94-3.01 (i, 3H) 3.02-3.14 (i, 1H) 3.16-3.38 (m, 3H) 3.38-3.51 (m, 1H) 3.61-3.78 (i, 2H) 3.80-4.07 (i, 58H) 4.37-4.66 (i, 3H) 4.72-4.81 (m, 1H) 5.12-5.23 (m, 3H) 5.44-5.55 (m, 1H) 7.81-7.99 (m, 4H) 8.11-8.20 (m, 1H) 8.61-8.69 (m, 1H) 8.82-8.89 (N, 1H) 8.90-8.99 (m, 1H) 9.24-9.29 (m, 1H).

The following compounds were prepared with procedures analogous to that described in Examples 10 and 11.

| | | LC-MS | |
|---|---|---|---|
| Ex. | Compound | m/z | NMR |
| 12 | 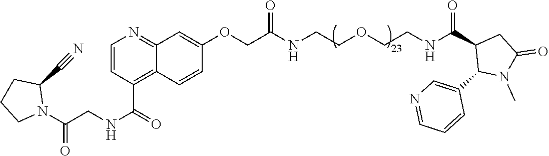 | (M + 2H)⁺⁺: 821.1 | ¹H NMR (400 MHz, DEUTERIUM OXIDE) δ ppm 2.38-2.48 (m, 5 H) 2.48-2.83 (m, 4 H) 2.96-3.02 (m, 3 H) 3.03-3.29 (m, 2 H) 3.39-3.51 (m, 1 H) 3.60-4.23 (m, 95 H) 5.12-5.22 (m, 4 H) 7.81-8.00 (m, 4 H) 8.13- 8.21 (m, 1 H) 8.65-8.67 (m, 1 H) 8.85-8.88 (m, 1 H) 8.93-9.00 (m, 1 H) 9.28 (d, J = 4.52 Hz, 1 H) |
| | N-(2-((S)-2-cyanopyrrolidin-1-yl)-2-oxoethyl)-7-((1-((2S,3S)- | | |
| | 1-methyl-5-oxo-2-(pyridin-3-yl)pyrrolidin-3-yl)-1,75-dioxo- | | |
| | 5,8,11,14,17,20,23,26,29,32,35,38,41,44,47,50,53,56,59, | | |
| | 62,65,68,71-tricosaoxa-2,74-diazahexaheptacontan-76- | | |
| | yl)oxy)quinoline-4-carboxamide | | |
| 13 | 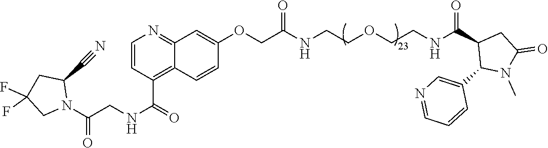 | (M + 2H)⁺⁺: 839.0 | ¹H NMR (400 MHz, DEUTERIUM OXIDE) δ ppm 2.37-2.45 (m, 8 H) 2.96-3.02 (m, 3 H) 3.04-3.50 (m, 5 H) 3.63-4.13 (m, 89 H) 4.37-4.66 (m, 3 H) 5.11-5.23 (m, 3 H) 5.48-5.55 (m, 1 H) 7.82-7.98 (m, 4 H) 8.13- 8.21 (m, 1 H) 8.61-8.69 (m, 1 H) 8.83-8.91 (m, 1 H) 8.91-9.04 (m, 1 H) 9.22-9.36 (m, 1 H) |
| | N-(2-((S)-2-cyano-4,4-difluoropyrrolidin-1-yl)-2-oxoethyl)-7- | | |
| | ((1-((2S,3S)-1-methyl-5-oxo-2-(pyridin-3-yl)pyrrolidin-3-yl)- | | |
| | 1,75-dioxo-5,8,11,14,17,20,23,26,29,32,35,38,41,44, | | |
| | 47,50,53,56,59,62,65,68,71-tricosaoxa-2,74- | | |
| | diazahexaheptacontan-76-yl)oxy)quinoline-4-carboxamide | | |

### Biological Assays

Example Compounds 1-13 which are compounds of Formulae (I) and (II) having a FAP binding moiety were tested in various biological assays as described in more detail below.

### Example 14: Antibody Dependent Cellular Cytotoxicity Reporter Assay

An antibody dependent cellular cytotoxicity (ADCC) reporter assay was conducted using the following four assay components: (i) ARM compound of Formula (I) targeting FAP (concentrations ranging from 1 μM to 10 μM); (ii) anti-cotinine antibody having a heavy chain sequence of SEQ ID NO: 11 and a light chain sequence of SEQ ID NO: 12 (rabbit variable region with human IgG1 Fc domain containing DE mutation (S239D/I332E)) (concentrations ranging from 0.01 μg/mL 200 μg/mL); (iii) Target cells: WI-38 cells (typically 1000-20,000 cells per well); and (iv) Reporter cells—Jurkat cells engineered to express FcγRIIIa with a reporter gene luciferase under the control of the NFAT promoter (typically 3000-75,000 cells per well). Reagents were combined in final volume of 20 μL in 384—well tissue culture treated plate. All four assay components were incubated together for about 12-18 hours. Thereafter, BioGlo Detection reagent (Promega) was added to the wells to lyse the cells and provide a substrate for the luciferase reporter protein.

Luminescence signal was measured on a microplate reader capable of measuring luminescence and signal:background was calculated by dividing the signal of a test well by the signal obtained when no compound of formula (I) was included in the assay. EC50 calculations were done using Graphpad Prism Software, specifically a nonlinear regression curve fit (Y=Bottom+(Top−Bottom)/(1+10\hat{ }((Log EC50−X)*HillSlope))).

ARMs compounds of Formula (I) were tested for ADCC activity in the above assay in one or more experimental runs and the results are shown in Table 3 below. Potency of the compounds of Formula (I) is reported as a pEC50 values. The pEC50 value is the negative log of the EC50 value, wherein the EC50 value is half maximal effective concentration measured in molar (M). For compounds tested in more than one experimental run, the pEC50 value is reported as an average.

TABLE 3
Results for ADCC Reporter Assay (Example 14)

| Compound Example No. | ADCC Reporter Assay pEC50 |
|---|---|
| 1 | 8.4 |
| 2 | Inactive¹ |
| 3 | Inactive |
| 4 | Inactive |
| 5 | Inactive |
| 6 | 7.9 |
| 7 | 8.3 |
| 8 | 8.4 |
| 9 | <6 |
| 10 | <6 |
| 11 | <6 |
| 12 | 9 |
| 13 | 9 |

¹Inactive = pEC50 < 5

SEQUENCE LISTINGS
Heavy chain CDR1 amino acid sequence
SEQ ID NO: 1
NYWMS
Heavy chain CDR2 amino acid sequence
SEQ ID NO: 2
DIHGNRGFNYHASWAKG
Heavy chain CDR3 amino acid sequence
SEQ ID NO: 3
ADDSGSHDI
Light chain CDR1 amino acid sequence
SEQ ID NO: 4
QSSQSVYSAKLS
Light chain CDR2 amino acid sequence
SEQ ID NO: 5
YGSTLAS
Light chain CDR3 amino acid sequence
SEQ ID NO: 6
QGTFYGPDWYFA
Variable heavy chain amino acid sequence
SEQ ID NO: 7
EVQLVESGGGLVQPGGSLRLSCAASGFTFSNYWMSWVRQAPGKGL
EWVGDIHGNRGFNYHASWAKGRFTVSRSKNTLYLQMNSLRAEDTA
VYYCAKADDSGSHDIWGQGTLVTVSS
Variable light chain amino acid sequence
SEQ ID NO: 8
DIQMTQSPSSLSASVGDRVTITCQSSQSVYSAKLSWYQQKPGKAP
KLLIYYGSTLASGVPSRFSGSGSGTQFTLTISSLQPEDFATYYCQ
GTFYGPDWYFAFGGGTKVEIK
Heavy chain amino acid sequence
SEQ ID NO: 9
EVQLVESGGGLVQPGGSLRLSCAASGFTFSNYWMSWVRQAPGKGL
EWVGDIHGNRGFNYHASWAKGRFTVSRSKNTLYLQMNSLRAEDTA
VYYCAKADDSGSHDIWGQGTLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKKVEPKSCDKTHTC
PPCPAPELLGGPDVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPEEKTISKAKGQPREPQVYTLPPSRDELTKN
QVSLTCLVKGFYPSDIAVEWESNGQPENNYKTTPPVLDSDGSFFL
YSKLTVDKSRWQQGNVFSCSVMHEALHNHYTQKSLSLSPGK
Light chain amino acid sequence
SEQ ID NO: 10
DIQMTQSPSSLSASVGDRVTITCQSSQSVYSAKLSWYQQKPGKAP
KLLIYYGSTLASGVPSRFSGSGSGTQFTLTISSLQPEDFATYYCQ
GTFYGPDWYFAFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy chain amino acid sequence
SEQ ID NO: 11
QQQLVESGGR LVTPGGSLTL TCTASGFSLN NYWMSWVRQA
PGKGLEWIGD IHGNRGFNYH ASWAKGRFTV SRTSTTVDLR
MTSLTTEDTA IYFCARADDS GSHDIWGPGT LVTVSSASTK
GPSVFPLAPS SKSTSGGTAA LGCLVKDYFP EPVTVSWNSG
ALTSGVHTFP AVLQSSGLYS LSSVVTVPSS SLGTQTYICN
VNHKPSNTKV DKKVEPKSCD KTHTCPPCPA PELLGGPDVF
LFPPKPKDTL MISRTPEVTC VVVDVSHEDP EVKFNWYVDG
VEVHNAKTKP REEQYNSTYR VVSVLTVLHQ DWLNGKEYKC
KVSNKALPAP EEKTISKAKG QPREPQVYTL PPSRDELTKN
QVSLTCLVKG FYPSDIAVEW ESNGQPENNY KTTPPVLDSD
GSFFLYSKLT VDKSRWQQGN VFSCSVMHEA LHNHYTQKSL
SLSPGK
Light chain amino acid sequence
SEQ ID NO: 12
ELDLTQTPSPVSAAVGDTVTINCQSSQSVYSAKLSWYQQKPGQPP
KLLIYYGSTLASGVPSREKGSGSGTQFSLTISDVQCADAATYYCQ
GTYYGPDWYFAFGGGTEVVVKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy chain amino acid sequence
SEQ ID NO: 13
QQQLVESGGRLVTPGGSLTLTCTASGFSLNNYWMSWVRQAPGKGL
EWIGDIHGNRGFNYHASWAKGRFTVSRTSTTVDLRMTSLTTEDTA
IYFCARADDSGSHDIWGPGTLVTVSSAKTTAPSVYPLAPVCGDTT
GSSVTLGCLVKGYFPEPVTLTWNSGSLSSGVHTFPAVLQSDLYTL
SSSVTVTSSTWPSQSITCNVAHPASSTKVDKKIEPRGPTIKPCPP
CKCPAPNLLGGPSVFIFPPKIKDVLMISLSPMVTCVVVDVSEDDP
DVQISWFVNNVEVLTAQTQTHREDYNSTLRVVSALPIQHQDWMSG
KEFKCKVNNKALPAPIERTISKPKGSVRAPQVYVLPPPEEEMTKK
QVTLTCMVTDFMPEDIYVEWTNNGKTELNYKNTEPVLDSDGSYFM
YSKLRVEKKNWVERNSYSCSVVHEGLHNHHTTKSFSRTPGK
Light chain amino acid sequence
SEQ ID NO: 14
ELDLTQTPSPVSAAVGDTVTINCQSSQSVYSAKLSWYQQKPGQPP
KLLIYYGSTLASGVPSREKGSGSGTQFSLTISDVQCADAATYYCQ
GTYYGPDWYFAFGGGTEVVVKRADAAPTVSIFPPSSEQLTSGGAS
VVCFLNNFYPKDINVKWKIDGSERQNGVLNSWTDQDSKDSTYSMS
STLTLTKDEYERHNSYTCEATHKTSTSPIVKSFNRNEC

1. A compound of Formula (I): 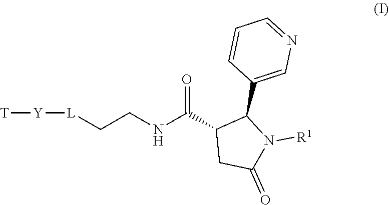
or a pharmaceutically acceptable salt thereof,
wherein: 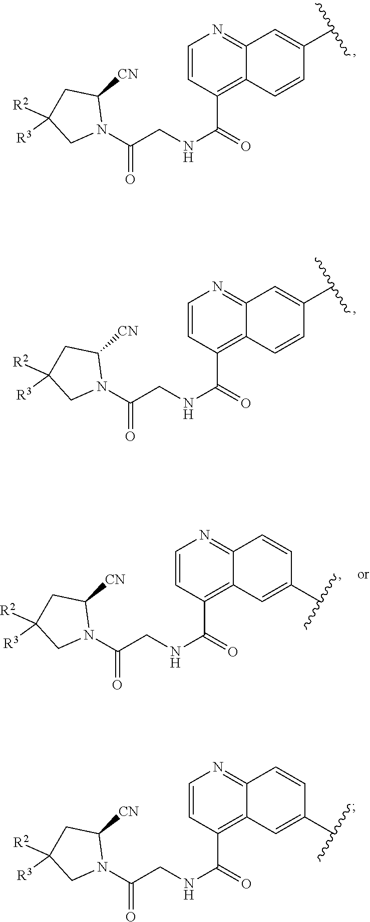
T is
R¹ is C₁₋₄ alkyl or C₃₋₆ cycloalkyl;
R² and R³ are each independently F or H;
L is a divalent linker of Formula (L-e), (L-p), (L-q), or (L-r): 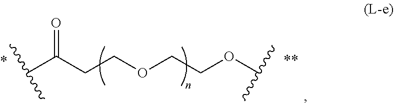
wherein n is an integer of 12 to 50; 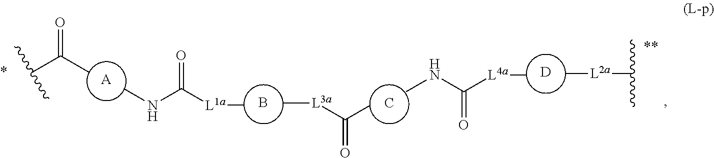
or a stereoisomer thereof,
wherein:
Ring A, Ring B, Ring C, and Ring D are each independently C₄₋₆ cycloalkylene;
L^{1a}, L^{3a}, and L⁴a are each independently C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—; 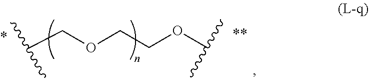
wherein n is an integer of 10 to 30; or 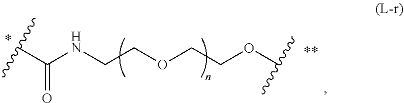
wherein n is an integer of 10 to 30;
wherein each 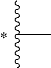
represents a covalent bond to the Y group of Formula (I), or when Y is a bond, a covalent bond to the T group of Formula (I), and each 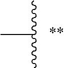
represents a covalent bond to the methylene group of Formula (I); and
Y is a bond or a divalent spacer moiety of one to twelve atoms in length.

2. The compound of claim 1, or a pharmaceutically acceptable salt thereof, wherein R¹ is —CH₃.

3. The compound of claim 1, or a pharmaceutically acceptable salt thereof, wherein both R² and R³ are F, or both R² and R³ are H.

4. The compound of claim 1, or a pharmaceutically acceptable salt thereof, wherein L is a divalent linker of Formula (L-p-i): 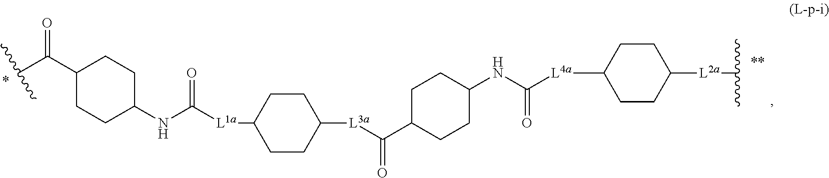
or a stereoisomer thereof,
wherein L^{1a}, L^{3a}, L^{4a}, L^{2a}, 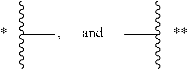
and are as defined for Formula (L-p).

5. The compound of claim 1, or a pharmaceutically acceptable salt thereof, wherein L is a divalent linker of Formula (L-p-ii): 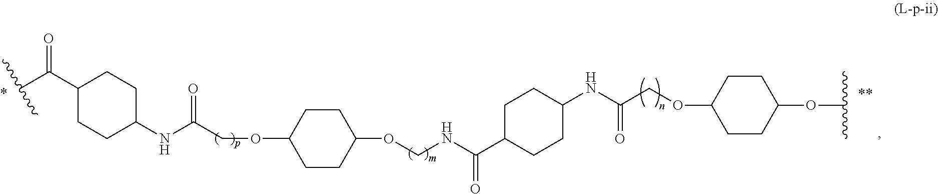
or a stereoisomer thereof,
wherein p is 1, 2, or 3; m is 1, 2, or 3; n is 1, 2, or 3; and 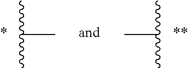
are as defined for Formula (L-p).

6. The compound of claim 1, or a pharmaceutically acceptable salt thereof, wherein L is a divalent linker of Formula (L-p) having the following structure:

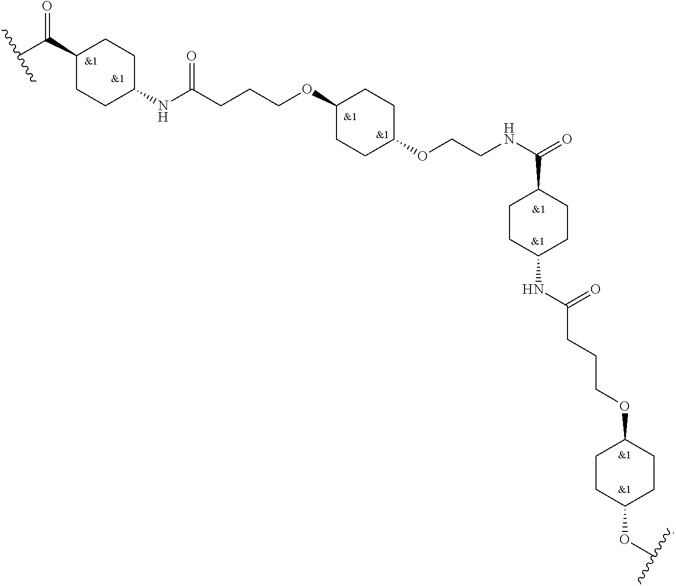 [SMILES: *O[C@H]1CC[C@H](OCCCC(=O)N[C@H]2CC[C@H](C(=O)NCCO[C@H]3CC[C@H](OCCCC(=O)N[C@H]4CC[C@H](C(*)=O)CC4)CC3)CC2)CC1]

7. The compound of claim 1, or a pharmaceutically acceptable salt thereof, wherein Y is selected from a bond; —NH—; —(C₁₋₁₂ alkylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene.

8. (canceled)

9. The compound of claim 1, or a pharmaceutically acceptable salt thereof, wherein Y is selected from the group consisting of:

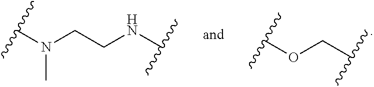

10. The compound of claim 1, wherein the compound is a compound in Table 1 or a pharmaceutically acceptable salt thereof.

11. A compound selected from the group consisting of: 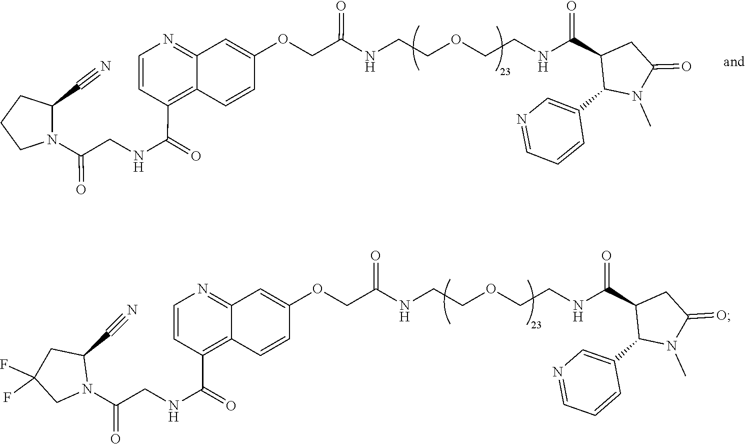
or a pharmaceutically acceptable salt thereof.

12. A method of treating and/or preventing a disease or disorder in a patient in need thereof, the method comprising: administering to the patient a therapeutically effective amount of the compound of claim 1 and an anti-cotinine antibody, or antigen-binding fragment thereof, wherein the disease or disorder is selected from a cancer.

13. (canceled)

14. The method of claim 12, wherein the disease is a cancer that is a solid tumor.

15. The method of claim 12, wherein the disease or disorder is a cancer selected from leukemia, lymphoma, lung cancer, hepatocellular carcinoma (HCC), colorectal cancer (CRC), cervical cancer, head and neck cancer, pancreatic cancer, prostate cancer, ovarian cancer, endometrial cancer, bladder cancer, bone cancer, esophageal cancer, gastric cancer, renal cancer, melanoma cancer, thyroid cancer, or breast cancer.

16. (canceled)

17. (canceled)

18. A method of increasing antibody-dependent cell cytotoxicity (ADCC) of FAP-expressing cells, the method comprising: contacting the cells with an effective amount of the compound of any one of claim 1 and an anti-cotinine antibody, or antigen-binding fragment thereof, wherein the FAP-binding moiety of the compound binds the FAP expressed on the cells.

19. A method of depleting FAP-expressing cells, the method comprising: contacting the cells with an effective amount of the compound of claim 1 and an anti-cotinine antibody, or antigen-binding fragment thereof, wherein the FAP-binding moiety of the compound binds the FAP expressed on the cells.

20. (canceled)

21. The method of claim 15, wherein the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

22. The method of claim 15, wherein the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8.

23. (canceled)

24. (canceled)

25. The method of claim 15, wherein the anti-cotinine antibody has a heavy chain comprising SEQ ID NO: 9 and a light chain comprising SEQ ID NO: 10.

26. A combination comprising the compound of claim 1 and an anti-cotinine antibody, or antigen-binding fragment thereof.

27. The combination of claim 26, wherein the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

28-31. (canceled)
